(19) United States
(12) Patent Application Publication
(10) Pub. No.: US 2025/0297289 A1
(43) Pub. Date: Sep. 25, 2025

(54) SYSTEMS AND METHODS FOR RNA-GUIDED DNA INTEGRATION

(71) Applicant: The Trustees of Columbia University in the City of New York, New York, NY (US)

(72) Inventors: Samuel Henry Sternberg, New York, NY (US); George Davis Lampe, New York, NY (US)

(21) Appl. No.: 19/230,907

(22) Filed: Jun. 6, 2025

Related U.S. Application Data
(63) Continuation of application No. PCT/US2023/082968, filed on Dec. 7, 2023.
(60) Provisional application No. 63/386,446, filed on Dec. 7, 2022.
(60) Provisional application No. 63/490,689, filed on Mar. 16, 2023.
(60) Provisional application No. 63/502,758, filed on May 17, 2023.

(51) Int. Cl. C12N 15/90; C12N 9/12; C12N 9/22; C12N 15/11

(52) U.S. Cl. CPC C12N 15/907; C12N 9/1241; C12N 9/226; C12N 15/111; C12N 2310/20; C12Y 207/07; C12Y 306/0401

(57) ABSTRACT

The present disclosure provides methods and systems for DNA modification and gene targeting comprising an engineered Clustered Regularly Interspaced Short Palindromic Repeats (CRISPR)-associated transposon (CAST) systems. More particularly, the present disclosure provides systems comprising: an engineered CAST system or one or more nucleic acids encoding the engineered CAST system, wherein the CAST system comprises at least one or both of: a) at least one Cas protein (e.g., Cas6, Cas7, Cas5, and/or Cas8) and b) one or more transposon-associated proteins (e.g., TnsA, TnsB, TnsC, TnsD, and/or TniQ), and at least one unfoldase protein (e.g., ClpX), or a nucleic acid encoding thereof. The present disclosure also provides systems, kits, and methods for nucleic acid modification in a cell.

### CROSS-REFERENCE TO RELATED APPLICATIONS

This application is a continuation of PCT International Application No. PCT/US2023/082968, filed Dec. 7, 2023, which claims the benefit of U.S. Provisional Application Nos. 63/386,446, filed Dec. 7, 2022, 63/490,689, filed Mar. 16, 2023, and 63/502,758, filed May 17, 2023, the contents of which are herein incorporated by reference in their entirety.

### STATEMENT REGARDING FEDERALLY SPONSORED RESEARCH OR DEVELOPMENT

This invention was made with government support under grant number HG011650 awarded by the National Institutes of Health. The government has certain rights in the invention.

### FIELD

The present disclosure relates to methods and systems for DNA modification and gene targeting comprising an engineered Clustered Regularly Interspaced Short Palindromic Repeats (CRISPR)-associated transposon (CAST) systems. Particularly, the present disclosure relates systems comprising: an engineered CAST system or one or more nucleic acids encoding the engineered CAST system, wherein the CAST system comprises at least one or both of: a) at least one Cas protein (e.g., Cas6, Cas7, Cas5, and/or Cas8) and b) one or more transposon-associated proteins (e.g., TnsA, TnsB, TnsC, TnsD, and/or TniQ), and at least one unfoldase protein (e.g., ClpX), or a nucleic acid encoding thereof.

### SEQUENCE LISTING STATEMENT

The content of the electronic sequence listing titled COLUM_41446_601_SequenceListing.xml (Size: 811,033 bytes; and Date of Creation: Dec. 7, 2023) is herein incorporated by reference in its entirety.

### BACKGROUND

CRISPR-Cas systems can be used for programmable DNA integration, in which the nuclease-deficient CRISPR-Cas machinery (either Cascade from Type I systems, or Cas12 from Type V systems) coordinates with Tn7 transposon-associated proteins to mediate RNA-guided DNA targeting and DNA integration, respectively. This activity may be leveraged in bacterial or eukaryotic cells for the targeted integration of user-defined genetic payloads at user-defined genomic loci, via a mechanism that obviates requirements for DNA double-strand breaks (DSBs) necessary for homology-directed repair.

### SUMMARY

Provided herein are systems for RNA-guided DNA modification.

In some embodiments, the systems comprise: a) an engineered Clustered Regularly Interspaced Short Palindromic Repeats (CRISPR)-associated transposon (CAST) system or one or more nucleic acids encoding the engineered CAST system, wherein the CAST system comprises at least one or all of: i) at least one Cas protein; ii) at least one transposon-associated protein; and iii) at least one guide RNA (gRNA) complementary to at least a portion of a target nucleic acid sequence; and b) an unfoldase protein, or a nucleic acid encoding thereof.

In some embodiments, the at least one Cas protein is derived from a Type I CRISPR-Cas system. In some embodiments, the engineered CRISPR-Tn system is a Type I-F system. In some embodiments, the at least one Cas protein comprises Cas5, Cas6, Cas7, and Cas8. In some embodiments, the at least one Cas protein comprises a Cas8-Cas5 fusion protein.

In some embodiments, the at least one Cas protein is derived from a Type V CRISPR-Cas system. In some embodiments, the engineered CRISPR-Tn system is a Type V-K system. In some embodiments, the at least one Cas protein comprises Cas12k.

In some embodiments, the at least one transposon protein is derived from a Tn7 or Tn7-like transposon system. In some embodiments, the at least one transposon-associated protein comprises TnsA, TnsB, TnsC, or a combination thereof. In some embodiments, the at least one transposon protein comprises a TnsA-TnsB fusion protein. In some embodiments, the at least one transposon-associated protein comprises TnsD and/or TniQ.

In some embodiments, the at least one gRNA is a non-naturally occurring gRNA. In some embodiments, the at least one gRNA is encoded in a CRISPR RNA (crRNA) array.

In some embodiments, the one or more nucleic acids encoding the engineered CAST system comprises one or more messenger RNAs, one or more vectors, or a combination thereof. In some embodiments, the at least one Cas protein, the at least one transposon-associated protein, and the at least one gRNA are encoded by different nucleic acids. In some embodiments, one or more of the at least one Cas protein, the at least one transposon-associated protein, and the at least one gRNA are encoded by a single nucleic acid.

In some embodiments, the at least one unfoldase protein comprises ClpX. In some embodiments, the at least one unfoldase protein is derived from same or different organism as that of the engineered CAST system.

In some embodiments, the nucleic acid encoding the at least one unfoldase protein (e.g., ClpX) comprises at least one messenger RNA, at least one vector, or a combination thereof. In some embodiments, the at least one unfoldase protein is encoded on a nucleic acid encoding one or more of: the at least one Cas protein, the at least one transposon-associated protein, and the at least one gRNA.

Also provided herein are compositions and cells comprising a present system. In some embodiments, the cell is a prokaryotic cell. In some embodiments, the cell is a eukaryotic cell (e.g., a mammalian cell, a human cell).

Further provided are methods for DNA integration comprising contacting a target nucleic acid sequence with a system or composition as disclosed herein.

In some embodiments, the target nucleic acid sequence is in a cell. In some embodiments, the contacting a target nucleic acid sequence comprises introducing the system into the cell. In some embodiments, the cell is a prokaryotic cell. In some embodiments, the cell is a eukaryotic cell (e.g., a mammalian cell, a human cell).

In some embodiments, introducing the system into the cell comprises administering the system to a subject. In some embodiments, the administering comprises in vivo administration. In some embodiments, the administering comprises transplantation of ex vivo treated cells comprising the system.

Other aspects and embodiments of the disclosure will be apparent in light of the following detailed description.

### BRIEF DESCRIPTION OF THE DRAWINGS

FIGS. 1A-1E show reconstitution of protein-RNA CAST components in human cells. FIG. 1A is a schematic detailing DNA integration using RNA-guided transposases. FIG. 1B shows Type I-F CRISPR-associated transposons encode the CRISPR RNA and seven proteins needed for DNA integration (top). Mammalian expression vectors used for heterologous reconstitution in human cells are shown at bottom. FIG. 1C shows western blotting with anti-FLAG antibody demonstrates robust protein expression upon individual (−) or multi-plasmid (+) co-transfection of HEK293T cells. Co-transfections contained all VchCAST components, with the FLAG-tagged subunit(s) indicated. β-actin was used as a loading control. Western blots were repeated in biological duplicates with similar results. FIG. 1D is a schematic of eGFP knockdown assay to monitor crRNA processing by Cas6 in HEK293T cells. Cleavage of the CRISPR direct repeat (DR)-encoded stem-loop severs the 5′-cap from the ORF and polyA (pA) tail, leading to a loss of eGFP fluorescence (bottom). FIG. 1E shows transposon-encoded VchCas6 (Type I-F3) exhibits efficient RNA cleavage and eGFP knockdown, as measured by flow cytometry. Knockdown was comparable to PseCas6 from a canonical CRISPR-Cas system (Type I-E), was absent with a non-cognate DR substrate, and was sensitive to C-terminal tagging. To control for over-expression, data were normalized to negative control conditions (−), in which dCas9 was co-transfected with the reporter. Data are shown as mean±s.d. for n=3 biologically independent samples.

FIGS. 2A-2G show development of QCascade and TnsC-based transcriptional activators to monitor DNA targeting. FIG. 2A is design of mammalian expression vectors encoding transposon-encoded Type I-F3 systems (VchQCascade). Cascade subunits are concatenated on a single polycistronic vector and connected by virally derived 2A peptides, as described previously. FIG. 2B is normalized mCherry fluorescence levels for the indicated experimental conditions, measured by flow cytometry. Whereas PseCascade stimulated robust activation, VchQCascade was inactive under these conditions. NT, non-targeting sgRNA/crRNA; T, targeting sgRNA/crRNA. FIG. 2C is design of separately encoded VchQCascade mammalian expression vectors with optimized NLS tag placement. FIG. 2D shows VchQCascade mediates transcriptional activation when encoded by re-engineered expression vectors, as measured by flow cytometry. mCherry expression is further enhanced when replacing mono-partite (SV40) NLS tags with bipartite (BP) NLS tags. NT, non-targeting; T, targeting. FIG. 2E is a schematic of transcriptional activation assay, in which DNA targeting by VchQCascade leads to multi-valent recruitment of VchTnsC-VP64. The assembly mechanism is based on recent biochemical, structural, and functional data. FIG. 2F is normalized mCherry fluorescence levels for the indicated experimental conditions, measured by flow cytometry. VchTnsC-based activation utilizes cognate protein-protein interactions, is dependent on the presence of TniQ, and involves ATP-dependent oligomer formation, which is eliminated with the E135A mutation. Several controls are shown for comparison, and guide RNAs target the same sites shown in FIG. 8A. NT, non-targeting crRNA. FIG. 2G shows transcriptional activation has strong sensitivity to RNA-DNA mismatches within both the PAM-proximal seed sequence and a PAM-distal region implicated in TnsC recruitment. Data are shown as in FIG. 2F, and the schematic at top displays the mismatched positions that were tested. Data were normalized to the perfectly matching (PM) crRNA. Data in FIGS. 2B, 2D and 2F-2G are shown as mean±s.d. for n=3 biologically independent samples.

FIGS. 3A-3E show potent genomic transcriptional activation via RNA-guided recruitment of the AAA+ ATPase, TnsC. FIG. 3A shows TnsC-VP64 directs efficient transcriptional activation of endogenous human gene expression, as measured by RT-qPCR. Four distinct crRNAs were combined for each condition and were either delivered individually, as a pool, or as a single multi-spacer multiplexed CRISPR array. The dCas9-VP64 and dCas9-VPR comparisons utilized four distinct sgRNAs encoded on separate plasmids. NT, non-targeting; T, targeting. FIG. 3B is a schematic demonstrating Cas6′s ability to process CRISPR arrays in vivo, thus allowing for the use of multiplexed CRISPR arrays to target multiple sites concurrently. FIG. 3C shows multiplexed activation of 4 distinct genes in the same cell pool. FIG. 3D is a 10 kb viewing window of ChIP-seq signal at the TTN promoter corresponding to TTN Guide 1. FIG. 3E is a differential binding analysis plot. Across consensus peaks for each condition, the only region exhibiting significantly different ChIP enrichment (FDR<0.05) between targeting and non-targeting conditions was the peak at the TTN promoter. Data in FIGS. 3A and 3C are shown as mean±s.d. for n=3 biologically independent samples. Viewing windows in FIG. 3D, are shown for 3 biologically independent targeting and non-targeting samples, and ChIP-seq signal is visualized as signal per million reads (SPMR). Data in e, is shown as the mean for n=3 biologically independent samples for each condition on the y axis, and the mean for all n=6 biologically independent samples on the x axis, irrespective of condition.

FIGS. 4A-4I show plasmid-based RNA-guided DNA integration in human cells using diverse CRISPR-associated transposases. FIG. 4A is a schematic of plasmid-to-plasmid transposition assay in human cells. FIG. 4B is Sanger sequencing confirmation of targeted integration products after plasmids isolation from human cells and selected in E. coli (FIG. 4A), showing the expected insertion site position and presence of target-site duplication (SEQ ID NO: 182 and 183, left and right side, respectively. FIG. 4C is a phylogenetic tree of Type I-F3 CRISPR-associated transposon systems, with labels of the homologs that were tested in human cells. FIG. 4D is a comparison of plasmid-to-plasmid integration efficiencies with eCAST-1 (VchCAST) and eCAST-2.1 (PseCAST), as measured by qPCR. Efficiencies are calculated by comparing Cq values between the integration junction product and a reference sequence located elsewhere on pTarget, as described in the Methods. FIG. 4E shows optimization of eCAST-2 (PseCAST) integration efficiencies by varying NLS placement and plasmid stoichiometries, etc., as described in FIG. 12, yielded an approximate 6-fold increase in integration efficiencies. FIG. 4F shows amplicon sequencing reveals a strong preference for integration 49-bp downstream of the 3′ edge of the site targeted by the crRNA in T-RL integrants. FIG. 4G shows deletion experiments confirmed the impact of each protein component, a targeting crRNA, and intact transposase active site (D220N mutation in TnsB, D458N mutation in TnsAB_{f}) for successful integration. FIG. 4H shows RNA-guided DNA integration functions with genetic payloads spanning 1-15 kb in size, transfected based on molar amount. FIG. 4I shows RNA-guided DNA integration has a strong sensitivity to mismatches across the entire 32-bp target site. Data were normalized to the perfectly matching (PM) crRNA, which exhibited an efficiency of 4.7±1.8%. Data in FIGS. 4D, 4E, 4G-4I are shown as mean±s.d. for n=3 biologically independent samples. Data in 4D, 4E, 4G-4I are determined by qPCR.

FIGS. 5A-5I show ClpX-mediated enhancement of genomic DNA integration with eCAST-3. FIG. 5A is Sanger sequencing (SEQ ID NO: 184) of nested PCR of genomic lysates in which eCAST-2.2 targeted the AAVS1 genome showing a junction product 49 bp downstream of the target site targeted by crRNA12 (AAVS1-1), one of the optimal crRNAs screened in FIG. 15A. FIG. 5B shows initial quantifications of genomic integration efficiencies at AAVS1-1. FIG. 5C shows integration efficiencies across multiple loci within human genome showed broadly limited efficiencies. Quantified integration efficiencies less than 0.0001% were not plotted, and “N.D.” represents a target site in which no integration events were detected across three biological replicates. FIG. 5D is proposed steps to facilitate successful targeted integration, including the downstream gap-repair for complete resolution of the integration product. FIG. 5E shows co-transfection of EcoClpX specifically improves genomic, but not plasmid, integration efficiencies in human cells. FIG. 5F shows co-transfecting EcoClpX at varied amounts directly impacts genomic integration efficiencies in human cells. FIG. 5G shows the impact of various Clp proteins from E. coli on genomic integration efficiencies in human cells. FIG. 5H shows integration efficiencies for samples before and after FACS of a fluorescent transfection marker to select for the top 20% brightest cells. Sorting enriched integration efficiencies, as measured by qPCR, ddPCR, and amplicon sequencing (see FIG. 14B). For amplicon sequencing samples, triangle data points represent all insertions characterized, while circle data points represent only 49-bp insertions. FIG. 5I shows integrations efficiencies investigated across multiple loci within the human genome with and without EcoClpX. Quantified integration efficiencies less than. 0.0001% were not plotted. Data in FIGS. 5B, 5C, 5E, 5G-5I are shown as mean±s.d. for n=3 biologically independent samples. Data in f are shown as mean for n=2 biologically independent samples. Data in FIGS. 5B, 5C, 5E, 5F-5I are quantified by amplicon sequencing.

FIGS. 6A-6D show improving expression and nuclear localization of VchCAST components. FIG. 6A is western blotting of various VchCAST components using distinct nuclear localization signals (NLS). Each component was appended with a 3× FLAG epitope tag and NLS tag, and nuclear fractionation was performed to separate nuclear and cytoplasmic cellular proteins. Histone deacetylase 1 (HDAC1) and a-Tubulin were used as nuclear- and cytoplasmic-specific loading controls, respectively. Western blots were repeated in biological duplicate with similar results. FIG. 6B is multiple fusion designs of TnsA and TnsB (TnsAB_{f}), with an NLS appended internally or at the N- or C-terminus. FIG. 5C is RNA-guided DNA integration activity determined in E. coli with the indicated TnsAB_{f} variants, as measured by qPCR. Data are shown as a mean±s.d. for n=3 biologically independent replicates. FIG. 5D is western blotting of TnsAB_{f} with internal NLS for validating expression and nuclear localization. The observed band was at the expected size, with no evidence of degradation or internal cleavage. Western blots were repeated in biological duplicate with similar results.

FIGS. 7A-7F show optimization of VchQCascade expression and transcriptional activation in human cells. FIG. 7A, top, is a schematic of mCherry reporter plasmid for transcriptional activation assays. The location of sites targeted by Cas9 single-guide RNAs (sgRNA) and Cascade CRISPR RNAs (crRNA) are indicated. PAMs are marked with a yellow circle. FIG. 7A, bottom, is a design of mammalian expression vectors encoding Cascade-based transcriptional activators from a Type I-E system (PseCascade), alongside dCas9-VP64 and dCas9-VPR controls. FIG. 7B is a depiction of V. cholerae TniQ-Cascade structure (PDB ID: 6PIF) showing the location of N- and C-termini in blue and red, respectively. All termini are solvent exposed and appear amenable to tagging. FIG. 7C is RNA-guided DNA integration activity in E. coli with the indicated NLS and/or 2A-tagged protein variants, measured by qPCR. Numerous tags have a deleterious effect. Data are normalized to the “WT no tags” condition, which resulted in a mean integration efficiency of 51±8%. FIG. 7D is RNA-guided DNA integration activity in E. coli with combined NLS and transcriptional activator fusions, as measured by qPCR. Fusing a VP64 or VPR transcriptional activator to the N-terminus of Cas7 exhibited the least deleterious effects on integration activity. Data are normalized to the “WT” condition, which resulted in a mean integration efficiency of 76.4%±4%. FIG. 7E is strength of transcriptional activation across a set of distinct crRNAs (“cr #”) targeting the mCherry reporter plasmid, as well as various activator-NLS constructs. Activation was measured using the reporter shown in FIG. 7A and measured by flow cytometry. S.V. indicates single vector design. Pc indicates polycistronic design of expression vectors as shown in FIG. 7A. FIG. 7D shows transcriptional activation by VchQCascade utilizing a VP64-Cas7 fusion construct is dependent on the presence of all Cascade components, as seen from the indicated dropout panel, but proceeds with ˜50% activity in the absence of TniQ. Data in FIGS. 7C-7F are shown as mean±s.d. for n=3 biologically independent samples.

FIGS. 8A-8E show optimization of TnsC-mediated transcriptional activation in human cells. FIG. 8A shows normalized mCherry fluorescence levels for the indicated experimental conditions, as measured by flow cytometry. VP64 was appended to TnsC at either the N- or C-terminus (VP64-TnsC or TnsC-VP64, respectively), and crRNAs (“cr #”) were cloned to target various sites upstream of the mCherry gene (top). mCherry fluorescence levels were measured by flow cytometry and normalized to the non-targeting gRNA condition (bottom). FIG. 8B shows transcriptional activation is affected by titrating the relative levels of each expression plasmid, with numbers below the graph indicating the fold-change of each plasmid amount relative to the initial stoichiometric condition with a targeting crRNA (second bar from left). mCherry fluorescence levels were measured by flow cytometry. FIG. 8C is a schematic showing the position of crRNAs (“cr #”) or sgRNAs (sg #) targeting each genomic locus for TnsC-mediated transcriptional activation for VchCAST (maroon) and dCas9 TTN activation (green). FIG. 8D is a representative schematic of multispacer crRNAs used during TnsC-mediated genomic transcriptional activation. For TTN, MIAT, and ASCL1, the 4 individual spacer sequences used in individual or pooled crRNA conditions were expressed as one multispacer CRISPR array. The CRISPR array is processed by Cas6 after transfection into cells and enables programmable targeting of multiple copies of QCascade and TnsC to a target locus. FIG. 8E is genomic transcriptional activation at the ACTC1 locus as quantified by RT-qPCR. 3 distinct crRNAs or gRNAs were used for each condition. Data in FIGS. 8A and 8B are shown as mean for n=2 biologically independent samples. Data in FIG. 8E, are shown as the mean±s.d. for n=3 biologically independent samples.

FIGS. 9A-9G show detection of TnsC recruitment to a genomic locus and profiling of off-target binding events. FIG. 9A is a 500 kb viewing window of ChIP-seq signal at the TTN promoter targeted by TTN Guide 1. FIG. 9B, top, is a 5 kb viewing window of ChIP-seq peak at the TTN promoter targeted by TTN Guide 1. FIG. 9B, bottom, 150 bp viewing window ChIP-seq peak at the TTN promoter targeted by TTN Guide 1. The peak summits in the targeting conditions align with the TTN promoter protospacer. FIG. 9C is a Venn diagram showing overlap of targeting and non-targeting peaks. FIG. 9D is a heatmap of signal intensity in a 2 kb window surrounding the peak center in TTN targeting exclusive peaks (1203), sorted in descending order by mean signal over the window. The peak with the highest mean signal was at the TTN promoter, which was targeted by TTN Guide 1. FIG. 9E is a heatmap of signal intensity in a 2 kb window surrounding the peak center in non-targeting (NT) exclusive peaks (2526), sorted in descending order by mean signal over the window. ChIP-seq signal was weak across NT exclusive peaks. FIG. 9F is a list of 5 genomic loci most similar to the TTN protospacer (SEQ ID NOs: 185-190, top to bottom). Mismatches at every 6th nucleotide, denoted by an “X”, were disregarded due to the nature with which Cas7 binds to crRNAs. All other mismatches are shown in red. FIG. 9G shows manual inspection of a 10 kb window surrounding each predicted off-target sequence. Minimal enrichment of ChIP-seq signal was seen in either the TTN targeting or the non-targeting condition. Viewing windows in FIGS. 9A, 9B, and 9G are shown for 3 biologically independent targeting and non-targeting samples, and ChIP-seq signal is visualized as signal per million reads (SPMR). Triangles in FIGS. 9A and 9G denote the position of either the expected TTN targeting sequence or of the predicted mismatch sequences.

FIGS. 10A-10E show detection and optimization of targeted integration using VchCAST (eCAST-1). FIG. 10A shows quantification of ChlorR resistant E. coli colonies after isolation from human cells. FIG. 10B is representative colony PCR of clonal integration products, detecting right transposon end (TnR) and left transposon end (TnL) junctions, as well as the KanR marker on the backbone of pTarget. Sanger sequencing of integration junctions are shown in FIG. 4B. This was repeated in biological duplicate with similar results. FIG. 10C is a nested PCR strategy to detect plasmid-transposon junctions directly from HEK293T cell lysates (left), and agarose gel electrophoresis showing target-cargo junction product bands (right). Expected amplicon sizes are marked for each PCR reaction with red arrows, and the crRNA was either non-targeting (NT) or targeting (T). “H₂O” denotes a condition in which the lysate was omitted from the PCR reactions. An aliquot of PCR-1 is used for PCR-2 such that a “nested PCR” is performed (see Methods). Sanger sequencing was performed on the product after PCR-2 in the targeting condition (SEQ ID NO: 191; bottom right). This was repeated in biological triplicate with similar results. FIG. 10D is a schematic of TaqMan probe strategy used to improve signal-to-noise by selectively detecting novel plasmid-transposon junctions. Probes labeled with FAM (blue) are used to detect target-transposon junctions, and probes labeled with SUN (green) are used to detect the target plasmid backbone (reference sequence), for integration efficiency quantification. Probes that span the junction of pTarget and the right transposon end of eCAST-1 are designed to anneal to an insertion event 49-bp downstream of the target site. FIG. 10E shows integration efficiencies were improved by varying the relative levels of pDonor, pTarget, or protein expression plasmids, as indicated; data were normalized to a control sample transfected with 100 ng of each component (left), or to the 100 ng condition for each varied protein (right), which had an average value of either 0.004% (left) or ranged from 0.0002-0.0005% (right), respectively. Data in e are shown as mean for n=2 biologically independent samples. Data shown in FIG. 10E were quantified via qPCR.

FIG. 11A-11E show systematic screening of homologous Type I-F CRISPR-associated transposons to uncover improved systems for mammalian cell applications. FIG. 11A is a cartoon depicting the multi-tiered approach that was applied to screen the indicated systems through a series of consecutive activity assays, with associated schematics shown for each functional assay. The middle panel depicts a transcriptional activation assay designed to monitor transposon DNA binding by TnsB in human cells using a tdTomato reporter plasmid. FIG. 11B is western blotting to detect expression of candidate Cas6 homologs in HEK293T cells, with or without human codon optimization (hCO), using monoclonal anti-FLAG M2 antibody; β-actin was used as a loading control. A range of expression levels were observed for human codon-optimized gene variants, and genes were poorly expressed for most systems when native bacterial coding sequences were used. FIG. 11C is activity assays for Cas6 homologs using the GFP knockdown assay shown in FIG. 1D. For each homolog, GFP fluorescence levels were measured by flow cytometry and normalized to the experimental condition in which the GFP reporter plasmid lacked a CRISPR direct repeat (DR) in the 5′-UTR. FIG. 11D is transcriptional activation data for TnsB-VP64 constructs from selected homologous CAST systems, as measured by flow cytometry. FIG. 11E is transcriptional activation data for QCascade and TnsC-VP64 from homologous CAST systems, as measured by flow cytometry. Tn7016, the final homolog that was selected for additional screening for transposition, is marked with a red arrow. Data in FIGS. 11C-11E are shown as mean for n=2 biologically independent samples.

FIGS. 12A-12I show parameter screening to further improve integration activity with the eCAST-2 (PseCAST) system. FIG. 12A is RNA-guided DNA integration efficiency for TnsAB fusion (TnsAB_{f}) protein design, with or without internal NLS, compared to the wild-type TnsA and TnsB proteins. Experiments were performed in E. coli, and efficiencies were measured by qPCR. FIG. 12B shows Tn7016 transposon ends were shortened relative to the constructs tested previously, generating the constructs indicated with red dashed boxes at the top. RNA-guided DNA integration activity was compared for the indicated transposon right end (RE) variants in E. coli, as measured by qPCR (bottom), while a 145 bp LE was used. The final pDonor design used in FIG. 4 contains 145-bp and 75-bp derived from the native left and right ends of Pseudoalteromonas Tn7016, respectively. FIG. 12C is agarose gel electrophoresis showing successful junction products from nested PCR (top) for eCAST-2, and Sanger sequencing chromatograms showing the expected integration distance (SEQ ID NO: 192; bottom). FIG. 12D shows integration efficiencies in HEK293T cells were similar using either typical or atypical CRISPR repeats, as measured by qPCR. FIG. 12E shows RNA-guided DNA integration activity compared with the indicated BP NLS tags on eCAST-2 components, as measured by qPCR. Individual components had their respective BP NLS tag repositioned from the N- to the C-terminus; “All” represents a condition in which all components had BP NLS tags on the noted terminus (left). Interestingly, the observed tag sensitivity is similar to, but distinct from, that with eCAST-1 components. Various combinations of N- and C-terminal NLS tagging for PseQCascade and PseTnsC (right). NT=non-targeting crRNA. FIG. 12F shows nuclear export signal (NES) predictions for eCAST-2 wild type (WT) and mutant TnsC (Mut). Predicted NES sequences were generated using NetNES (WT=SEQ ID NO: 193; Mut=SEQ ID NO: 194). FIG. 12G shows RNA-guided DNA integration activity was compared after appending additional NLS tags on PseTnsC and removing a potential internal nuclear export signal (NES) sequence with the mutations L255A, L258V, and L260V, as indicated in FIG. 12F. FIG. 12H shows RNA-guided DNA integration activity compared after varying the relative levels of individual eCAST-2 protein and RNA expression plasmids. Data were measured by qPCR and were normalized to either the sample transfected with 100 ng of each component for each condition, with an average integration efficiency of 0.10-0.17% (left), or a control sample (labeled T) transfected with the standard eCAST-2 plasmid amounts, as detailed in the Methods section with an average integration efficiency of 2.7% (right). FIG. 12I is a plasmid-based BxbI recombination assay performed to benchmark eCAST-2 integration efficiency to other commonly used large DNA insertion tools. Data in FIGS. 12A, 12B, 12D, and 12I are shown as the mean±s.d. for n=3 biologically independent samples. Data in FIGS. 12E, 12G, and 12H are shown as the mean for n=2 biologically independent samples.

FIGS. 13A-13E show selection, seeding, and sorting strategies result in further increases in eCAST-2.2 integration efficiencies. FIG. 13A is normalized RNA-guided DNA integration efficiency for eCAST-2.2 in the absence or presence of puromycin selection, and after harvesting cells from between 2-6 days post-transfection. Experiments used a puromycin resistance plasmid as a transfection selection marker, in addition to eCAST-2.2 component plasmids, and integration activity was measured by qPCR and normalized to the condition harvested on day 3 without puromycin selection, which had an average integration efficiency of 2.3%. FIG. 13B shows eCAST-2.2 integration efficiencies as a function of seeding density 24 hours before transfection. 24-well plates were with various cell densities ranging from 1×10³ to 2×10⁵ cells per well, and integration activity was measured by qPCR. FIG. 13C shows transfection of HEK293T cells via various cationic lipid delivery methods affected integration efficiencies. FIG. 13D is a schematic showing the use of a GFP transfection marker and cell sorting to increase integration efficiency. A GFP expression plasmid was transfected in significantly smaller amounts relative to eCAST-2.2 component plasmids, and cells were sorted into bins of varying GFP expression levels. FIG. 13E shows eCAST-2.2 integration efficiencies are enhanced after using flow cytometry to sort cells for the brightest GFP positive cells. Cells were sorted four days after transfection, and the top 20% brightest cells were binned in increments of 5%, with Bin 1 representing the top 5% brightest cells and Bin 4 representing the 15-20% brightest cells. Integration efficiencies were determined for each bin separately, or for the unsorted population, as measured by qPCR. Integration efficiencies were normalized to the unsorted, targeting crRNA condition, which had a value of 0.44%. Data in FIG. 13A are shown as the mean of n=2 biologically independent samples. Data in FIGS. 13B, 13C, and 13E are shown as the mean±s.d. for n=3 biologically independent samples.

FIGS. 14A-14D show eCAST-2.2 integration is biased towards T-RL insertion and reproducibly quantified across distinct approaches. FIG. 14A shows RNA-guided DNA integration is heavily biased towards insertion in the right-left (T-RL) orientation, with only a small minority of insertion events occurring in the left-right (T-LR) orientation. Integration efficiencies were calculated using SYBR qPCR. Triangle data points represent integration events in the T-LR orientation, while circle data points represent integration events in the T-RL orientation. FIG. 14B is a comparison of different strategies to detect and quantify integration efficiencies. For next-generation amplicon sequencing, a variant pDonor was constructed in which a primer binding site that is also present at the target site is cloned within the transposon cargo at a distance from the transposon right end (R), such that unedited sites and integration products yield amplicons of indistinguishable length using pF and pR primers (top). Consequently, next-generation sequencing of these amplicons provides relative abundances of edited and unedited alleles in the population, allowing for higher sensitivity in detecting integration efficiencies. For qPCR and ddPCR detection strategies, Taqman probes and primers are designed to amplify either the integration product or a reference sequence used to calculate integration efficiencies (bottom). For plasmid-based integration assays, the reference sequence is a distinct sequence on the plasmid target (pTarget). FIG. 14C is representative agarose gel electrophoresis demonstrating identical amplicon products for non-targeting (NT) and targeting (T) samples after PCR-1 for NGS analysis. This was repeated in biological triplicates with similar results. FIG. 14D is calculated integration efficiencies for the same experimental samples, measured by TaqMan qPCR, droplet digital PCR (ddPCR), and amplicon deep sequencing. ddPCR and qPCR analyses specifically probe for integration products that are 49-bp downstream of the target site, whereas amplicon sequencing analysis does not impose the same stringent distance bias, allowing the quantification of integration products within a larger window surrounding the anticipated integration site. Editing efficiencies for both eCAST-2.2 and eCAST-1 were consistent between different quantification methods. For amplicon sequencing samples, triangle data points represent all insertions characterized, while circle data points represent only 49-bp insertions. Data in FIG. 14A, are shown as the mean±s.d. for n=3 biologically independent samples. Data in FIG. 14C are shown as the mean for n=2 biologically independent samples.

FIGS. 15A-15F show possible improvements to eCAST-2.2 genomic integration activity and identification of kinetic bottlenecks. FIG. 15A shows a unique target site was cloned into a modified pTarget, in which the downstream integration site sequence remained the same, allowing investigation of the impact of different crRNA sequences on integration efficiencies (left). Cloning various target sites into the modified pTarget that correspond to target sites within the AAVS1 safe harbor locus enabled screening of crRNAs to identify active sequences (right). Efficiencies were normalized to the crRNA used in plasmid-targeting assays, which had an average integration efficiency of 2.0%. FIG. 15B shows simplification of transfection workflow via polycistronic expression of QCascade, and genomic integration efficiencies with different constructs. “Separate Vectors” represents a condition in which TniQ, Cas8, Cas7, and Cas6 were all expressed from separate pcDNA3.1-like vectors. FIG. 15C shows the impact of additional NLS tags on eCAST-2 QCascade components on genomic integration efficiencies. All QCascade components had a singular NLS tag, unless noted. FIG. 15D shows the impact of stably-expressed eCAST-2 components on genomic integration efficiencies. Cell lines were generated via Sleeping Beauty with drug selection, and various components were stably expressed (indicated by operons shown on the y-axis). “All components transfected” represents conditions in which all eCAST-2 components were co-transfected, while “Remaining components transfected” represents conditions in which only the non-expressed eCAST-2 components were transfected. FIG. 15E shows the impact of co-transfection of E. coli Integration Host Factor (IHF) on human genomic integration efficiencies. “T+scIHF” represents a condition in which a plasmid expressing a single-chain IHFa/b was co-transfected with a targeting gRNA. FIG. 15F shows varying cell harvest day and selection of transfected cells based on a concurrent drug marker improves integration efficiencies, although overall efficiencies remain low. Data in FIGS. 15A-15E are shown as mean for n=2 biologically independent samples. Data in FIG. 15G are shown as the mean±s.d. for n=3 biologically independent samples. Data in FIG. 15A was determined by qPCR. Data in FIGS. 15B-15F were determined by amplicon sequencing.

FIGS. 16A-16D show genomic editing outcomes with ClpX. FIG. 16A shows mutational analysis of ClpX-mediated editing improvements. Point mutations were designed to either ablate ATP hydrolysis (E185Q and R370K) or perturb substrate engagement (Y153A and V154F). FIG. 16B shows the impact of native ClpX proteins on eCAST-2 and eCAST-1. PseClpX and VchClpX improved eCAST-2 and eCAST-1 genomic integration efficiencies, respectively, but EcoClpX consistently produces a more robust improvement. FIG. 16C shows human-derived ClpX does not improve genomic integration efficiencies for eCAST-2. The putative mitochondrial targeting sequence from human derived ClpX was replaced with a BP-NLS tag. FIG. 16D shows the proposed model for the role of ClpX in improving genomic integration efficiencies. In the absence of ClpX, the PTC is sufficiently stable to prevent accessibility to the DNA intermediate, leading to a loss of genomic integration events. In contrast, inclusion of ClpX facilitates unfolding of CAST components, resulting in destabilization/dissociation of the complex and accessibility to the DNA intermediate. Data in FIGS. 16A-16C are shown as the mean±s.d. for n=3 biologically independent samples.

FIGS. 17A-17G show engineering CAST systems with ClpX. FIG. 17A shows the impact of atypical spacer lengths on plasmid-based integration efficiencies (the canonical spacer length, 32nt, is marked with a maroon triangle). FIG. 17B shows the impact of 32nt vs 33nt spacer lengths on genomic integration efficiencies at the AAVS1-1 target site. Two different crRNAs were tested that were nearby in the genomic locus, minimizing disruption of potential downstream integration-site requirements. FIG. 17C shows the impact of encoding the crRNA on the pDonor for genomic integration efficiencies. The U6 promoter, crRNA, and U6 terminator sequences were cloned on either a separate plasmid or in the pDonor backbone. FIG. 17D shows genomic integration as a function of different cationic lipid transfection methods FIG. 17E is a comparison of integration efficiencies in the presence and absence of ClpX as measured by qPCR, ddPCR, and amplicon sequencing for AAVS1-1; ddPCR and amplicon sequencing for OXA1L-2. For amplicon sequencing samples, triangle data points represent all insertions characterized, while circle data points represent only 49-bp insertions. FIG. 17F shows varying cell harvest day and selection of transfected cells based on a concurrent drug marker improves integration efficiencies, in the presence of ClpX. FIG. 17G is a schematic of sequences that were analyzed to understand if undesirable editing outcomes were occurring with eCAST-3. If a sequence did not contain a transposon end, the sequence surrounding the intended integration site was investigated for a higher frequency of indel events compared to samples in which a non-targeting crRNA was used. If a transposon end was detected in the sequence, the sequence was analyzed for additional mutations. Lower left shows mutations surrounding the integration region at AAVS1-1 do not occur above background frequencies present when a NT crRNA is co-transfected. Right hand side shows mutations upstream the integration site at AAVS1-1 do not occur at a higher rate compared to WT alleles (top). Mutations in the transposon end and surrounding the target site duplication at AAVS1-1 do not occur at rates above background sequencing error (bottom). Integration events at the major integration site (49 bp downstream of crRNA) were analyzed. Data in FIGS. 17A-17C and 17E (for AAVS1-1) are shown as mean for n=2 biologically independent samples. Data in FIGS. 17D, 17E (for OXA1L-2), 17F, and 17G are shown as mean±s.d. for n=3 biologically independent samples. Data were quantified by amplicon sequencing.

FIGS. 18A and 18B show leveraging eCAST-3 to perform targeted RNA-guided DNA integration at multiple target sites. FIG. 18A shows an exemplary workflow for applying eCAST-3 to new target sites. First, potential targets with CC PAMs are identified in region of interest. Target sites are then screened for optimal primers for amplicon sequencing. The downstream primer binding site is cloned into a pDonor immediately adjacent to the RE, enabling NGS-based quantification. Cells are then transfected with pCRISPR, pQCascade, pTnsAB, pTnsC, pClpX, pDonor, and an optional drug selection marker. After 4 days, cells can be harvested for PCR prep and subsequent NGS-based analysis. FIG. 18B is representative integration site distributions for transfections shown in FIG. 5I. The length of the spacer is shown, and the distance represents the length from the PAM-distal end of the spacer to the transposon end.

FIGS. 19A and 19B show PseCAST integration efficiencies with extra-chromosomal and chromosomal DNA substrates. FIG. 19A shows integration efficiencies of PseCAST when the target DNA substrate is varied. When the crRNA targets a DNA sequence that is encoded within the genome, integration efficiencies drop approximately two to three orders of magnitude efficiencies between plasmid and genomic substrates. Genomic-based integration transfections targeted the AAVS1 safe harbor locus within intron 1 of the PPP1R12C gene. FIG. 19B is a schematic of potential rate-limiting steps that uniquely impact episomal and genomic integration assays. Notably, episomal DNA does not need to undergo DNA replication, and thus dissociation and gap repair of the post-transposition complex is optional. Genomic DNA undergoes replication, thus an unresolved post-transposition complex may result in toxicity or activation of complex DNA repair pathways.

FIG. 20 is a schematic of CAST-based integration events resulting in DNA intermediates requiring host proteins for complete resolution. Transposase machineries mediate excision of transposon from donor plasmid and insertion into target site, resulting in a gapped intermediate containing 5′ DNA overhangs. In order for complete gap repair and resolution of the transposition event, transposase proteins must dissociate from the target site to allow host repair factors to access and repair intermediate substrates.

FIG. 21 is a graph of titrations of ClpX expression plasmid showing a dose-dependent correlation of genomic integration efficiencies in the presence of ClpX. As the amounts of a pcDNA3.1 plasmid expressing E. coli derived ClpX is increased, genomic integration efficiencies increase. At 100 ng of ClpX plasmid transfected, improvements in integration efficiencies are saturated. Density of cells transfected approximately 24 hours prior to transfection has little effect on overall integration efficiencies in the presence of ClpX. Genomic-based integration transfections targeted the AAVS1 safe harbor locus within intron 1 of the PPP1R12C gene.

FIG. 22 shows ClpX improves genomic integration efficiencies at multiple target sites across the genome through integration assays with PseCAST machinery with and without ClpX. Each transfection contained a crRNA expression plasmid targeting a unique site across the human genome.

FIG. 23 shows that ClpX does not improve other genomic editing methods. Cas9-mediated genome editing was performed with and without ClpX in human cells, and the frequency of indels were quantified. The region surrounding the sequence targeted by gRNA was PCR-amplified and analyzed via next-generation sequencing and CRISPResso2 (Clement, Nat Biotechnol 37, (2019)). Genomic-based editing transfections targeted the AAVS1 safe harbor locus within intron 1 of the PPP1R12C gene.

FIG. 24 shows the characterization of functional residues within the C-terminus of TnsB. Serial truncations of TnsB show immediate ablation of plasmid-based integration efficiencies. Pleitropic residues may reside in the C-terminus of TnsB, interacting with both TnsC and ClpX at different stages of the CAST integration pathway.

### DETAILED DESCRIPTION

The disclosed systems, kits, and methods provide systems and methods for nucleic acid integration utilizing engineered CRISPR-associated transposon systems. The disclosed systems, kits, and methods provide systems and methods for RNA-guided DNA integration utilizing engineered CRISPR-associated transposon systems.

Tn7-like and Tn5053-like transposons that encode nuclease-deficient CRISPR-Cas systems, also known as CRISPR-transposons (CRISPR-Tn) and CRISPR-associated transposons (CAST), catalyze the Insertion of Transposable Elements by Guide RNA-Assisted TargEting (sometimes referred to as INTEGRATE, or INTEGRATE technology). Here CAST activity is shown using two diverse systems from V. cholerae and Pseudoalteromonas, demonstrating that the same molecular determinants of RNA-guided transposition hold true in bacteria and eukaryotes. Also, a strategy for targeted recruitment of an oligomeric transposase component, TnsC for use in transcriptional activation at levels similar to conventional dCas9-based reagents was developed. Further, RNA-guided DNA integration is simulated in mammalian cells using an unfoldase protein (e.g., ClpX). The ATP-dependent Clp protease ATP-binding subunit ClpX, hereafter referred to as ClpX, together with obligate protein RNA components catalyze site-specific, RNA-guided insertion of mini-transposon DNA payloads into genomic target sites, leading to an enhancement of the observed integration efficiencies by one or more orders of magnitude across multiple tested target sites. Given the roles of ClpX in mechanically unfolding post-integration strand-transfer complexes, also known as transpososomes, ClpX may find utility in the disclosed systems and method for the removal of CAST machinery from genomic target sites after the integration reaction, thereby rendering those sites accessible to DNA repair machinery for gap fill-in and DNA ligation.

Section headings as used in this section and the entire disclosure herein are merely for organizational purposes and are not intended to be limiting.

### Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. As used herein, comprising a certain sequence or a certain SEQ ID NO usually implies that at least one copy of said sequence is present in recited peptide or polynucleotide. However, two or more copies are also contemplated. The singular forms “a,” “and” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. For example, any nomenclature used in connection with, and techniques of cell and tissue culture, molecular biology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein, “nucleic acid” or “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41(14): 4503-4510 (2002)) and U.S. Pat. No. 5,034,506), locked nucleic acid (LNA; see Wahlestedt et al., Proc. Natl. Acad. Sci. U.S.A., 97: 5633-5638 (2000)), cyclohexenyl nucleic acids (see Wang, J. Am. Chem. Soc., 122: 8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

Nucleic acid or amino acid sequence “identity,” as described herein, can be determined by comparing a nucleic acid or amino acid sequence of interest to a reference nucleic acid or amino acid sequence. A number of mathematical algorithms for obtaining the optimal alignment and calculating identity between two or more sequences are known and incorporated into a number of available software programs. Examples of such programs include CLUSTAL-W, T-Coffee, and ALIGN (for alignment of nucleic acid and amino acid sequences), BLAST programs (e.g., BLAST 2.1, BL2SEQ, and later versions thereof) and FASTA programs (e.g., FASTA3x, FAS™, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106(10): 3770-3775 (2009), Durbin et al., eds., Biological Sequence Analysis: Probabilistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)).

The term “homology” and “homologous” refers to a degree of identity. There may be partial homology or complete homology. A partially homologous sequence is one that is less than 100% identical to another sequence.

As used herein, the term “hybridization” is used in reference to the pairing of complementary nucleic acids. Hybridization and the strength of hybridization (e.g., the strength of the association between the nucleic acids) is influenced by such factors as the degree of complementary between the nucleic acids, stringency of the conditions involved, and the Tₘ of the formed hybrid. Hybridization methods involve the annealing of one nucleic acid to another, complementary nucleic acid, e.g., a nucleic acid having a complementary nucleotide sequence. The ability of two polymers of nucleic acid containing complementary sequences to find each other and “anneal” or “hybridize” through base pairing interaction is a well-recognized phenomenon. The initial observations of the “hybridization” process by Marmur and Lane, Proc. Natl. Acad. Sci. USA, 46: 453 (1960) and Doty et al., Proc. Natl. Acad. Sci. USA, 46: 461 (1960), have been followed by the refinement of this process into an essential tool of modern biology. For example, hybridization and washing conditions are now well known and exemplified in Sambrook et al., supra. The conditions of temperature and ionic strength determine the “stringency” of the hybridization.

As used herein, a “double-stranded nucleic acid” may be a portion of a nucleic acid, a region of a longer nucleic acid, or an entire nucleic acid. A “double-stranded nucleic acid” may be, e.g., without limitation, a double-stranded DNA, a double-stranded RNA, a double-stranded DNA/RNA hybrid, etc. A single-stranded nucleic acid having secondary structure (e.g., base-paired secondary structure) and/or higher order structure (e.g., a stem-loop structure) may also be considered a “double-stranded nucleic acid.” For example, triplex structures are considered to be “double-stranded.” In some embodiments, any base-paired nucleic acid is a “double-stranded nucleic acid.”

The term “gene” refers to a DNA sequence that comprises control and coding sequences necessary for the production of an RNA having a non-coding function (e.g., a ribosomal or transfer RNA), a polypeptide, or a precursor of any of the foregoing. The RNA or polypeptide can be encoded by a full length coding sequence or by any portion of the coding sequence so long as the desired activity or function is retained. Thus, a “gene” refers to a DNA or RNA, or portion thereof, that encodes a polypeptide or an RNA chain that has functional role to play in an organism. For the purpose of this disclosure, it may be considered that genes include regions that regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The terms “non-naturally occurring,” “engineered,” and “synthetic” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

A “vector” or “expression vector” is a replicon, such as plasmid, phage, virus, or cosmid, to which another DNA segment, e.g., an “insert,” may be attached or incorporated so as to bring about the replication of the attached segment in a cell.

A cell has been “genetically modified,” “transformed,” or “transfected” by exogenous DNA, e.g., a recombinant expression vector, when such DNA has been introduced inside the cell. The presence of the exogenous DNA results in permanent or transient genetic change. The transforming DNA may or may not be integrated (covalently linked) into the genome of the cell. For example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods and compositions provided herein, the mammal is a human.

The term “contacting” as used herein refers to bring or put in contact, to be in or come into contact. The term “contact” as used herein refers to a state or condition of touching or of immediate or local proximity. Contacting a composition to a target destination, such as, but not limited to, an organ, tissue, cell, or tumor, may occur by any means of administration known to the skilled artisan.

As used herein, the terms “providing,” “administering,” and “introducing,” are used interchangeably herein and refer to the placement of the systems of the disclosure into a cell, organism, or subject by a method or route which results in at least partial localization of the system to a desired site. The systems can be administered by any appropriate route which results in delivery to a desired location in the cell, organism, or subject.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### Systems

Disclosed herein are systems or kits for DNA modification comprising: a) an engineered Clustered Regularly Interspaced Short Palindromic Repeats (CRISPR)-CRISPR associated (Cas) transposon (CAST) system or one or more nucleic acids encoding the engineered CAST system, wherein the CAST system comprises at least one or all of: i) at least one Cas protein; ii) at least one transposon-associated protein; and iii) a guide RNA (gRNA) complementary to at least a portion of a target nucleic acid sequence; and, optionally, b) at least one unfoldase protein, or a nucleic acid encoding thereof. In some embodiments, one or more of the at least one Cas protein are part of a ribonucleoprotein complex with the gRNA.

The system may be a cell free system. Also disclosed is a cell comprising the system described herein. In some embodiments, the cell is a prokaryotic cell. In some embodiments, the cell is a eukaryotic cell. In some embodiments, the cell is a mammalian cell (e.g., a cell of a non-human primate or a human cell). Thus, in some embodiments, disclosed herein are systems or kits for DNA integration into a target nucleic acid sequence in a eukaryotic cell (e.g., a mammalian cell, a human cell).

a. CAST System

CRISPR-Cas systems are currently grouped into two classes (1-2), six types (I-VI) and dozens of subtypes, depending on the signature and accessory genes that accompany the CRISPR array. The engineered CAST system may be derived from a Class 1 CRISPR-Cas system or a Class 2 CRISPR-Cas system.

Type I CRISPR-Cas systems encode a multi-subunit protein-RNA complex called Cascade, which utilizes a crRNA (or guide RNA) to target double-stranded DNA during an immune response. Cascade itself has no nuclease activity, and degradation of targeted DNA is instead mediated by a trans-acting nuclease known as Cas3. In Type I-A and I-D systems, the activities of Cas3 are carried out by separate proteins called Cas3′ (helicase) and Cas3″ (nuclease). Type I-D systems also comprise Cas10d instead of Cas8.

The engineered CAST system may be derived from a Type I CRISPR-Cas system (such as subtypes I-B and I-F, including I-F variants). In some embodiments, the engineered CAST system is a Type I-F system. In some embodiments, the engineered CAST system is a Type I-F3 system.

On the other hand, type V systems belong to the Class 2 CRISPR-Cas systems, characterized by a single-protein effector complex that is programmed with a gRNA. The transposon-associated Type V CRISPR-Cas systems may be derived from: Anabaena variabilis ATCC 29413 (or Trichormus variabilis ATCC 29413 (see GenBank CP000117.1)), Cyanobacterium aponinum IPPAS B-1202, Filamentous cyanobacterium CCP2, Nostoc punctiforme PCC 73102, and Scytonema hofmannii PCC 7110. Type V systems comprise Cas12k, previously known as C2c5.

In some embodiments, the engineered CAST system is derived from Vibrio cholerae, Photobacterium iliopiscarium, Vibrio parahaemolyticus, Pseudoalteromonas sp., Pseudoalteromonas ruthenica, Photobacterium ganghwense, Shewanella sp., Vibrio diazotrophicus, Vibrio sp. 16, Vibrio sp. F12, Vibrio splendidus, Aliivibrio wodanis, Aliivibrio sp., Endozoicomonas ascidiicola, and Parashewanella spongiae.

In some embodiments, the system comprises components from different CAST systems. In some embodiments, one or more of the at least one Cas protein and one or more transposon-associated proteins may be derived from a homologous CRISPR-transposon system compared to the other protein components in the system. In some embodiments, the engineered CAST system is at least partially derived (e.g., contains one or more Cas protein or transposon-associated protein) from any one or more of: Vibrio cholerae, Photobacterium iliopiscarium, Vibrio parahaemolyticus, Pseudoalteromonas sp., Pseudoalteromonas ruthenica, Photobacterium ganghwense, Shewanella sp., Vibrio diazotrophicus, Vibrio sp. 16, Vibrio sp. F12, Vibrio splendidus, Aliivibrio wodanis, Aliivibrio sp., Endozoicomonas ascidiicola, and Parashewanella spongiae.

In some embodiments, the system comprises two or more engineered CAST systems. Pairing of orthogonal systems with their orthogonal donor DNA substrates enables tandem insertion of multiple distinct payloads directly adjacent to each other without any risk of repressive effects from target immunity. For example, one, two, three, four, five, or more orthogonal CAST systems may be used. In some embodiments, multiple orthogonal RNA-guided transposases and their transposon donor DNAs may be integrated into distal regions of a given chromosome or genome, such that the lack of sequence identity between the transposon ends of the distinct transposon DNA substrates prevents genetic instability and the risk of recombination.

In some embodiments, the engineered CAST system comprises Cas5, Cas6, Cas7, Cas8, or any combination thereof. In some embodiments, the engineered CAST system comprises Cas8-Cas5 fusion protein.

An engineered CAST system of the present invention may comprise one or more transposon-associated proteins (e.g., transposases or other components of a transposon). The transposon-associated proteins may facilitate recognition or cleavage of the target nucleic acid and subsequent insertion of the donor nucleic acid into the target nucleic acid.

In some embodiments, the transposon-associated proteins are derived from a Tn7 or Tn7-like transposon. Tn7 and Tn7-like transposons may be categorized based on the presence of the hallmark DDE-like transposase gene, tnsB (also referred to as tniA), the presence of a gene encoding a protein within the AAA+ ATPase family, tnsC (also referred to as tniB), one or more targeting factors that define integration sites (which may include a protein within the tniQ family, also referred to as tnsD, but sometimes includes other distinct targeting factors), and inverted repeat transposon ends that typically comprise multiple binding sites thought to be specifically recognized by the TnsB transposase protein. In Tn7, the targeting factors, or “target selectors,” comprise the genes tnsD and tnsE. Based on biochemical and genetics studies, it is known that TnsD binds a conserved attachment site in the 3′ end of the glmS gene, directing downstream integration, whereas TnsE binds the lagging strand replication fork and directs sequence-non-specific integration primarily into replicating/mobile plasmids.

The most well-studied member of this family of transposons is Tn7, hence why the broader family of transposons may be referred to as Tn7-like. “Tn7-like” term does not imply any particular evolutionary relationship between Tn7 and related transposons; in some cases, a Tn7-like transposon will be even more basal in the phylogenetic tree and thus Tn7 can be considered as having evolved from, or derived from, this related Tn7-like transposon.

Whereas Tn7 comprises tnsD and tnsE target selectors, related transposons comprise other genes for targeting. For example, Tn5090/Tn5053 encode a member of the tniQ family (a homolog of E. coli tnsD) as well as a resolvase gene tniR; Tn6230 encodes the protein TnsF; and Tn6022 encodes two uncharacterized open reading frames orf2 and orf3; Tn6677 and related transposons encode variant Type I-F and Type I-B CRISPR-Cas systems that work together with TniQ for RNA-guided mobilization; and other transposons encode Type V-U5 CRISPR-Cas systems that work together with TniQ for random and RNA-guided mobilization. Any of the above transposon systems are compatible with the systems and methods described herein.

In some embodiments, the one or more transposon-associated proteins comprise TnsA, TnsB, TnsC, or a combination thereof. In some embodiments, the one or more transposon-associated proteins comprise TnsB and TnsC. In some embodiments, the one or more transposon-associated proteins comprise TnsA, TnsB, and TnsC.

In some embodiments, the at least one transposon protein comprises a TnsA-TnsB fusion protein. TnsA and TnsB can be fused in any orientation: N-terminus to C-terminus; C-terminus to N-terminus; N-terminus to N-terminus; or C-terminus to C-terminus, respectively. Preferably the C-terminus of TnsA is fused to the N-terminus of TnsB.

In some embodiments, the TnsA-TnsB fusion may be fused using an amino acid linker peptide of various lengths to provide greater physical separation and allow more spatial mobility between the fused portions. The linker may comprise any amino acids and may be of any length. In some embodiments, the linker may be less than about 50 (e.g., 40, 30, 20, 10, or 5) amino acid residues.

In some embodiments, the linker is a flexible linker, such that TnsA and TnsB can have orientation freedom in relationship to each other. For example, a flexible linker may include amino acids having relatively small side chains, and which may be hydrophilic. Without limitation, the flexible linker may contain a stretch of glycine and/or serine residues. In some embodiments, the linker comprises at least one glycine-rich region. For example, the glycine-rich region may comprise a sequence comprising [GS]n, wherein n is an integer between 1 and 10.

In some embodiments, the linker further comprises a nuclear localization sequence (NLS). The NLS may be embedded within a linker sequence, such that it is flanked by additional amino acids. In some embodiments, the NLS is flanked on each end by at least a portion of a flexible linker. In some embodiments, the NLS is flanked on each end by a glycine rich region of the linker. Suitable nuclear localization sequences for use with the disclosed system are described further below and are applicable to use with the TnsA-TnsB fusion protein. In some embodiments, the linker comprises the amino acid sequence of GCGCGKRTADGSEFESPKKKRKVGSGSGG (SEQ ID NO: 168).

In some embodiments, the disclosed systems further comprise TnsD, TniQ, or a combination thereof or a nucleic acid encoding TnsD, TniQ, or a combination thereof. Thus, the one or more transposon-associated proteins may comprise TnsD, TniQ, or a combination thereof.

In some embodiments, the engineered CAST system comprises TnsA, TnsB, TnsC, TnsD and TniQ. In some embodiments, the engineered CAST system comprises Cas5, Cas6, Cas7, Cas8, TnsA, TnsB, TnsC, and at least one or both of TnsD or TniQ. In certain embodiments, the engineered CAST system comprises TnsD. In certain embodiments, the engineered CAST system comprises TniQ. In certain embodiments, the engineered CAST system comprises TnsD and TniQ.

In some embodiments, any combination of the at least one Cas protein and the at least one transposon associated protein may be expressed as a single fusion protein. In some embodiments, each of the at least one Cas protein and one or more of the at least one transposon-associated protein are part of a single fusion protein in which the components are expressed as a single megapeptide.

Sequences of exemplary Cas proteins, transposon-associated proteins, gRNAs, and transposon ends can also be found in International Patent Applications WO2020181264, WO2022261122, and WO2022266492 incorporated herein by reference.

In some embodiments, at least one of the one or more Cas protein comprises: a Cas6 protein comprising an amino acid sequence having at least 70% (e.g., having at least 75%, at least 80%, at least 85%, at least 90%, at least 92%, at least 95%, at least 96%, at least 97%, at least 98%, at least 99%, or 100%) identity to SEQ ID NO: 207 or 208; a Cas7 protein comprising an amino acid sequence having at least 70% (e.g., having at least 75%, at least 80%, at least 85%, at least 90%, at least 92%, at least 95%, at least 96%, at least 97%, at least 98%, at least 99%, or 100%) identity to SEQ ID NO: 205 or 206; or a Cas8-Cas5 fusion protein comprising an amino acid sequence having at least 70% (e.g., having at least 75%, at least 80%, at least 85%, at least 90%, at least 92%, at least 95%, at least 96%, at least 97%, at least 98%, at least 99%, or 100%) identity to SEQ ID NO: 203 or 204.

In some embodiments, at least one of the one or more transposon-associated proteins comprises: a TnsA protein comprising an amino acid sequence having at least 70% (e.g., having at least 75%, at least 80%, at least 85%, at least 90%, at least 92%, at least 95%, at least 96%, at least 97%, at least 98%, at least 99%, or 100%)) identity to SEQ ID NO: 195 or 196; a TnsB protein comprising an amino acid sequence having at least 70% (e.g., having at least 75%, at least 80%, at least 85%, at least 90%, at least 92%, at least 95%, at least 96%, at least 97%, at least 98%, at least 99%, or 100%)) identity to SEQ ID NO: 197 or 198; a TnsC protein comprising an amino acid sequence having at least 70% (e.g., having at least 75%, at least 80%, at least 85%, at least 90%, at least 92%, at least 95%, at least 96%, at least 97%, at least 98%, at least 99%, or 100%)) identity to SEQ ID NO: 199 or 200; or a TniQ protein comprising an amino acid sequence having at least 70% (e.g., having at least 75%, at least 80%, at least 85%, at least 90%, at least 92%, at least 95%, at least 96%, at least 97%, at least 98%, at least 99%, or 100%)) identity to SEQ ID NO: 201 or 202.

The invention is not limited to the disclosed or referenced exemplary sequences. Indeed, genetic sequences can vary between different strains, and this natural scope of allelic variation is included within the scope of the invention.

In other embodiments, any of the proteins described or referenced herein may comprise a sequence corresponding to, or substantially corresponding to, the wild-type version of the protein. For example, the sequence may substantially correspond to the wild-type protein sequence except for changes made for facile cloning or removal of known restriction sites. Thus, protein products from potential alternative start codons compared to the predicted nucleic acid sequences in this document are therefore not excluded.

Any of the proteins described or referenced herein may comprise one or more amino acid substitutions as compared to the recited sequences. An amino acid “replacement” or “substitution” refers to the replacement of one amino acid at a given position or residue by another amino acid at the same position or residue within a polypeptide sequence. Amino acids are broadly grouped as “aromatic” or “aliphatic.” An aromatic amino acid includes an aromatic ring. Examples of “aromatic” amino acids include histidine (H or His), phenylalanine (F or Phe), tyrosine (Y or Tyr), and tryptophan (W or Trp). Non-aromatic amino acids are broadly grouped as “aliphatic.” Examples of “aliphatic” amino acids include glycine (G or Gly), alanine (A or Ala), valine (V or Val), leucine (L or Leu), isoleucine (I or He), methionine (M or Met), serine (S or Ser), threonine (T or Thr), cysteine (C or Cys), proline (P or Pro), glutamic acid (E or Glu), aspartic acid (A or Asp), asparagine (N or Asn), glutamine (Q or Gin), lysine (K or Lys), and arginine (R or Arg).

The amino acid replacement or substitution can be conservative, semi-conservative, or non-conservative. The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz and Schirmer, Principles of Protein Structure, Springer-Verlag, New York (1979)). According to such analyses, groups of amino acids may be defined where amino acids within a group exchange preferentially with each other, and therefore resemble each other most in their impact on the overall protein structure (Schulz and Schirmer, supra). Examples of conservative amino acid substitutions include substitutions of amino acids within the sub-groups described above, for example, lysine for arginine and vice versa such that a positive charge may be maintained, glutamic acid for aspartic acid and vice versa such that a negative charge may be maintained, serine for threonine such that a free-OH can be maintained, and glutamine for asparagine such that a free —NH₂ can be maintained. “Semi-conservative mutations” include amino acid substitutions of amino acids within the same groups listed above, but not within the same sub-group. For example, the substitution of aspartic acid for asparagine, or asparagine for lysine, involves amino acids within the same group, but different sub-groups. “Non-conservative mutations” involve amino acid substitutions between different groups, for example, lysine for tryptophan, or phenylalanine for serine, etc.

The components of the system may be present in the system in various ratios. In some embodiments, each of the protein components or the nucleic acids encoding thereof are provided in a 1:1 ratio. For example, when each protein component is encoded on a single nucleic acid, the single nucleic acid comprises a single coding sequence for each protein component.

In some embodiments, any one of the protein components may be provided in greater abundance to any other protein component. In certain embodiments, Cas7 or the nucleic acid encoding Cas7 in greater abundance compared to the remaining protein components or nucleic acids encoding thereof. For example, multiple copies of a nucleic acid encoding Cas7 may be provided for each copy of any of the other components (e.g., Cas6, Cas5, Cas8, TniQ or TnsC). In some embodiments, Cas7 is encoded on a nucleic acid separate from any of the other components such that it can be provided in the system and methods herein at a higher abundance or dosage than the other components. Analogously, higher concentrations of the Cas7 protein can be provided in the systems and methods compared to the other proteins. In some embodiments, for every one copy of Cas6 or Cas8, or nucleic acids encoding thereof, 2 or more copies of Cas7 or a nucleic acid encoding Cas7 are included in the system. In some embodiments, for every one copy of Cas6 or Cas8 or nucleic acids encoding thereof, 5-10 copies of Cas7 or a nucleic acid encoding Cas7 are included in the system.

b. gRNA

In some embodiments, the engineered CAST systems further comprise a gRNA complementary to at least a portion of the target nucleic acid sequence, or a nucleic acid encoding the at least one gRNA.

The gRNA may be a crRNA, crRNA/tracrRNA (or single guide RNA, sgRNA). The terms “gRNA,” “guide RNA,” “crRNA,” and “CRISPR guide sequence” may be used interchangeably throughout and refer to a nucleic acid comprising a sequence that determines the binding specificity of the engineered CAST system. A gRNA hybridizes to (complementary to, partially or completely) a target nucleic acid sequence (e.g., the genome in a host cell). In some embodiments, the at least one gRNA is encoded in a CRISPR RNA (crRNA) array.

The system may further comprise a target nucleic acid. In some embodiments, target nucleic acid sequence comprises a human sequence.

The gRNA or portion thereof that hybridizes to the target nucleic acid (a target site) may be between 15-40 nucleotides in length. In some embodiments, the gRNA sequence that hybridizes to the target nucleic acid is 15, 16, 17, 18, 19, 20, 21, 22, 23, 24, 25, 26, 27, 28, 29, 30, 31, 32, 33, 34, 35, 36, 37, 38, 39, or 40 nucleotides in length. gRNAs or sgRNA(s) used in the present disclosure can be between about 5 and 100 nucleotides long, or longer (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91 92, 93, 94, 95, 96, 97, 98, 99, or 100 nucleotides in length, or longer).

To facilitate gRNA design, many computational tools have been developed (See Prykhozhij et al. (PLOS ONE, 10(3): (2015)); Zhu et al. (PLOS ONE, 9(9) (2014)); Xiao et al. (Bioinformatics. January 21 (2014)); Heigwer et al. (Nat Methods, 11(2): 122-123 (2014)). Methods and tools for guide RNA design are discussed by Zhu (Frontiers in Biology, 10 (4) pp 289-296 (2015)), which is incorporated by reference herein. Additionally, there are many publicly available software tools that can be used to facilitate the design of sgRNA(s); including but not limited to, Genscript Interactive CRISPR gRNA Design Tool, WU-CRISPR, and Broad Institute GPP sgRNA Designer. There are also publicly available pre-designed gRNA sequences to target many genes and locations within the genomes of many species (human, mouse, rat, zebrafish, C. elegans), including but not limited to, IDT DNA Predesigned Alt-R CRISPR-Cas9 guide RNAs, Addgene Validated gRNA Target Sequences, and GenScript Genome-wide gRNA databases.

In addition to a sequence that binds to a target nucleic acid, in some embodiments, the gRNA may also comprise a scaffold sequence (e.g., tracrRNA). In some embodiments, such a chimeric gRNA may be referred to as a single guide RNA (sgRNA). Exemplary scaffold sequences will be evident to one of skill in the art and can be found, for example, in Jinek, et al. Science (2012) 337(6096):816-821, and Ran, et al. Nature Protocols (2013) 8:2281-2308, incorporated herein by reference in their entireties.

In some embodiments, the gRNA sequence does not comprise a scaffold sequence and a scaffold sequence is expressed as a separate transcript. In such embodiments, the gRNA sequence further comprises an additional sequence that is complementary to a portion of the scaffold sequence and functions to bind (hybridize) the scaffold sequence.

As described elsewhere herein the protein and gRNA components of the system may be expressed and transcribed from the nucleic acids using any promoter or regulatory sequences known in the art. In some embodiments, the gRNA is transcribed under control of an RNA Polymerase II promoter. In some embodiments, the gRNA is transcribed under control of an RNA Polymerase III promoter.

In some embodiments, the gRNA sequence is at least 50%, 55%, 60%, 65%, 70%, 75%, 80%, 85%, 90%, 95%, 96%, 97%, 98%, 99%, or at least 100% complementary to a target nucleic acid. In some embodiments, the gRNA sequence is at least 50%, 55%, 60%, 65%, 70%, 75%, 80%, 85%, 90%, 95%, 96%, 97%, 98%, 99%, or at least 100% complementary to the 3′ end of the target nucleic acid (e.g., the last 5, 6, 7, 8, 9, or 10 nucleotides of the 3′ end of the target nucleic acid).

The gRNA may be a non-naturally occurring gRNA.

The system may further comprise a target nucleic acid. The target nucleic acid may be flanked by a protospacer adjacent motif (PAM). A PAM site is a nucleotide sequence in proximity to a target sequence. For example, PAM may be a DNA sequence immediately following the DNA sequence targeted by the engineered CAST system.

The target sequence may or may not be flanked by a protospacer adjacent motif (PAM) sequence. In certain embodiments, a nucleic acid-guided nuclease can only cleave a target sequence if an appropriate PAM is present, see, for example Doudna et al., Science, 2014, 346(6213): 1258096, incorporated herein by reference. A PAM can be 5′ or 3′ of a target sequence. A PAM can be upstream or downstream of a target sequence. In one embodiment, the target sequence is immediately flanked on the 3′ end by a PAM sequence. A PAM can be 1, 2, 3, 4, 5, 6, 7, 8, 9, 10 or more nucleotides in length. In certain embodiments, a PAM is between 2-6 nucleotides in length. The target sequence may or may not be located adjacent to a PAM sequence (e.g., PAM sequence located immediately 3′ of the target sequence) (e.g., for Type I CRISPR/Cas systems). In some embodiments, e.g., Type I systems, the PAM is on the alternate side of the protospacer (the 5′ end). Makarova et al. describes the nomenclature for all the classes, types, and subtypes of CRISPR systems (Nature Reviews Microbiology 13:722-736 (2015)). Guide structures and PAMs are described in by R. Barrangou (Genome Biol. 16:247 (2015)).

Non-limiting examples of the PAM sequences include: CC, CA, AG, GT, TA, AC, CA, GC, CG, GG, CT, TG, GA, AGG, TGG, T-rich PAMs (such as TTT, TTG, TTC, etc.), NGG, NGA, NAG, NGGNG and NNAGAAW (W=A or T), NNNNGATT, NAAR (R=A or G), NNGRR (R=A or G), NNAGAA, and NAAAAC, where N is any nucleotide. In some embodiments, the PAM may comprise a sequence of CN, in which N is any nucleotide. In select embodiments, the PAM may comprise a sequence of CC.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule, which can form hydrogen bonds (e.g., Watson-Crick base pairing) with a second nucleic acid sequence. Full complementarity is not necessarily required, provided there is sufficient complementarity to cause hybridization. There may be mismatches distal from the PAM.

In some embodiments, when the system comprises TnsA, TnsB, TnsC, TnsD and TniQ binding to the target nucleic acid may be mediated through a TnsD binding site within the target nucleic acid sequence. Thus, the recognition of the target nucleic acid utilizing the systems described herein may proceed in a gRNA-dependent and/or-independent manner.

c. Unfoldase

The present systems may further include at least one unfoldase protein. Unfoldases are proteins that catalyze the unfolding of a native protein without affecting the primary structure. The unfoldase may be an NTP driven unfoldase. NTP driven unfoldases may include ATP-dependent proteases, including, but not limited to, ATPases, AAA proteases, or AAA+ enzymes (e.g., AAA+ enzyme). In some embodiments, the at least one unfoldase protein may comprise ClpX (caseinolytic mitochondrial matrix peptidase chaperone subunit X). In some embodiments, the at least one unfoldase protein may comprise a homolog of ClpX.

ClpX homologs may be readily screened through systematic testing and optimization of a large panel of homologs, identified through bioinformatic search strategies such as BLASTp and psi-BLASTp. In some embodiments, the unfoldase protein (e.g., ClpX) is derived from the same host organism as that of the engineered CAST system. In some embodiments, the unfoldase protein (e.g., ClpX) is derived from a different host organism as that of the engineered CAST system. As such, the at least one unfoldase protein (e.g., ClpX) is not limited from which organism it is derived. In some embodiments, the unfoldase protein (e.g., ClpX) is derived from the E. coli genome. In other embodiments, the unfoldase protein (e.g., ClpX) from the cognate strain from which the engineered CAST system is derived. For example, the unfoldase protein from Vibrio cholerae HE-45 can be used alongside RNA-guided DNA integration machinery derived from Tn6677, while unfoldase proteins from Pseudoalteromonas sp. S983 can be used alongside RNA-guided DNA integration machinery derived from Tn7016. In some embodiments, the ClpX is selected from the proteins shown in Table 1, or homologs thereof. In some embodiments, the ClpX comprises an amino acid sequence having at least 70% similarity to any of SEQ ID NOs: 1-8.

d. Nuclear Localization Sequence

In the systems disclosed herein, one or more of the at least one Cas protein, the at least one transposon-associated protein, or the unfoldase protein (e.g., ClpX) may comprise a nuclear localization signal (NLS). The nuclear localization sequence may be appended to the one or more of the at least one Cas protein, the at least one transposon-associated protein and the unfoldase protein (e.g., ClpX) at a N-terminus, a C-terminus, embedded in the protein (e.g., inserted internally within the open reading frame (ORF)), or a combination thereof.

In some embodiments, one or more of the at least one Cas protein, the at least one transposon-associated protein, and the at least one unfoldase protein (e.g., ClpX) comprises two or more NLSs. The two or more NLSs may be in tandem, separated by a linker, at either end terminus of the protein, or embedded in the protein (e.g., inserted internally within the ORF instead).

The nuclear localization sequence may comprise any amino acid sequence known in the art to functionally tag or direct a protein for import into a cell's nucleus (e.g., for nuclear transport). Usually, a nuclear localization sequence comprises one or more positively charged amino acids, such as lysine and arginine.

In some embodiments, the NLS is a monopartite sequence. A monopartite NLS comprise a single cluster of positively charged or basic amino acids. In some embodiments, the monopartite NLS comprises a sequence of K-K/R-X-K/R, wherein X can be any amino acid. Exemplary monopartite NLS sequences include those from the SV40 large T-antigen, c-Myc, and TUS-proteins.

In some embodiments, the NLS is a bipartite sequence. Bipartite NLSs comprise two clusters of basic amino acids, separated by a spacer of about 9-12 amino acids. Exemplary bipartite NLSs include the NLS of nucleoplasmin, KR[PAATKKAGQA]KKKK (SEQ ID NO: 169), and the NLS of EGL-13, MSRRRKANPTKLSENAKKLAKEVEN (SEQ ID NO: 170). In some embodiments, the NLS comprises a bipartite SV40 NLS. In certain embodiments, the NLS comprises an amino acid sequence having at least 70% similarity to KRTADGSEFESPKKKRKV (SEQ ID NO: 171). In select embodiments, the NLS comprises, consists essentially of, or consists of an amino acid sequence of KRTADGSEFESPKKKRKV (SEQ ID NO: 171).

The protein components of the disclosed system (e.g., the Cas proteins, the transposon-associated proteins, or the unfoldase protein (e.g., ClpX)) may further comprise an epitope tag (e.g., 3× FLAG tag, an HA tag, a Myc tag, and the like). In some embodiments, the epitope tag may be adjacent, either upstream or downstream, to a nuclear localization sequence. The epitope tags may be at the N-terminus, a C-terminus, or a combination thereof of the corresponding protein.

e. Donor Nucleic Acid

The system may further include a donor nucleic acid to be integrated. The donor nucleic acid may be a part of a bacterial plasmid, bacteriophage, a virus, autonomously replicating extra chromosomal DNA element, linear plasmid, linear DNA, linear covalently closed DNA, mitochondrial or other organellar DNA, chromosomal DNA, and the like. In some embodiments, the donor nucleic acid comprises a cargo nucleic acid sequence.

The donor nucleic acid may be flanked by at least one transposon end sequence. In some embodiments, the donor nucleic acid is flanked on the 5′ and the 3′ end with a transposon end sequence. The term “transposon end sequence” refers to any nucleic acid comprising a sequence capable of forming a complex with the transposase enzymes thus designating the nucleic acid between the two ends for rearrangement. Usually, these sequences contain inverted repeats and may be about 10-150 base pairs long, however the exact sequence requirements differ for the specific transposase enzymes. Transposon ends sequences may or may not include additional sequences that promotes or augment transposition.

The transposon end sequences on either end may be the same or different. The transposon end sequence may be the endogenous CRISPR-transposon end sequences or may include deletions, substitutions, or insertions. The endogenous CRISPR-transposon end sequences may be truncated. In some embodiments, the transposon end sequence includes an about 40 base pair (bp) deletion relative to the endogenous CRISPR-transposon end sequence. In some embodiments, the transposon end sequence includes an about 100 base pair deletion relative to the endogenous CRISPR-transposon end sequence. The deletion may be in the form of a truncation at the distal (in relation to the cargo) end of the transposon end sequences.

The donor nucleic acid, and by extension the cargo nucleic acid, may of any suitable length, including, for example, about 50-100 bp (base pairs), about 100-1000 bp, at least or about 10 bp, at least or about 20 bp, at least or about 25 bp, at least or about 30 bp, at least or about 35 bp, at least or about 40 bp, at least or about 45 bp, at least or about 50 bp, at least or about 55 bp, at least or about 60 bp, at least or about 65 bp, at least or about 70 bp, at least or about 75 bp, at least or about 80 bp, at least or about 85 bp, at least or about 90 bp, at least or about 95 bp, at least or about 100 bp, at least or about 200 bp, at least or about 300 bp, at least or about 400 bp, at least or about 500 bp, at least or about 600 bp, at least or about 700 bp, at least or about 800 bp, at least or about 900 bp, at least or about 1 kb (kilobase pair), at least or about 2 kb, at least or about 3 kb, at least or about 4 kb, at least or about 5 kb, at least or about 6 kb, at least or about 7 kb, at least or about 8 kb, at least or about 9 kb, at least or about 10 kb, or greater.

f. Nucleic Acids

The one or more nucleic acids encoding the engineered CAST system or the nucleic acid encoding the unfoldase protein (e.g., ClpX) may be any nucleic acid including DNA, RNA, or combinations thereof. In some embodiments, nucleic acids comprise one or more messenger RNAs, one or more vectors, or any combination thereof.

The at least one Cas protein, the at least one transposon-associated protein, the at least one unfoldase protein (e.g., ClpX), the at least one gRNA, and the donor nucleic acid may be on the same or different nucleic acids (e.g., vector(s)). In some embodiments, the at least one Cas protein, the at least one transposon associated protein, and the unfoldase protein (e.g., ClpX) are encoded by different nucleic acids. In some embodiments, the at least one Cas protein and the at least one transposon associated protein encoded by a single nucleic acid. In some embodiments, the at least one Cas protein, the at least one transposon associated protein, and the at least one unfoldase protein (e.g., ClpX) are encoded by a single nucleic acid. In some embodiments, the at least one gRNA is encoded by a nucleic acid different from the nucleic acid(s) encoding the at least one Cas protein, the at least one transposon associated protein, and the at least one unfoldase protein (e.g., ClpX). In some embodiments, the at least one gRNA is encoded by a nucleic acid also encoding the at least one Cas protein, the at least one transposon associated protein, the at least one unfoldase protein (e.g., ClpX), or a combination thereof. In some embodiments, the nucleic acid encoding the at least one Cas protein, at least one transposon associated protein, the at least one unfoldase protein (e.g., ClpX), the at least one gRNA, or any combination thereof further comprises the donor nucleic acid.

In select embodiments, a single nucleic acid encodes the gRNA and at least one Cas protein. The gRNA may be encoded anywhere in the nucleic acid encoding the at least one Cas protein. In some embodiments, the gRNA is encoded in the 3′ UTR of the Cas protein-coding gene.

In certain embodiments, engineering the system for use in eukaryotic cells may involve codon-optimization. It will be appreciated that changing native codons to those most frequently used in mammals allows for maximum expression of the system proteins in mammalian cells (e.g., human cells). Such modified nucleic acid sequences are commonly described in the art as “codon-optimized,” or as utilizing “mammalian-preferred” or “human-preferred” codons. In some embodiments, the nucleic acid sequence is considered codon-optimized if at least about 60% (e.g., 65%, 70%, 75%, 80%, 85%, 90%, 95%, or 98%) of the codons encoded therein are mammalian preferred codons. Furthermore, in some embodiments, engineering the CRISPR-Cas system involves incorporating elements of the native CRISPR array into the disclosed system.

The present disclosure also provides for DNA segments encoding the proteins and nucleic acids disclosed herein, vectors containing these segments and cells containing the vectors. The vectors may be used to propagate the segment in an appropriate cell and/or to allow expression from the segment (e.g., an expression vector). The person of ordinary skill in the art would be aware of the various vectors available for propagation and expression of a nucleic acid sequence.

The present disclosure further provides engineered, non-naturally occurring vectors and vector systems, which can encode one or more or all of the components of the present system. The vector(s) can be introduced into a cell that is capable of expressing the polypeptide encoded thereby, including any suitable prokaryotic or eukaryotic cell.

The vectors of the present disclosure may be delivered to a eukaryotic cell in a subject. Modification of the eukaryotic cells via the present system can take place in a cell culture, where the method comprises isolating the eukaryotic cell from a subject prior to the modification. In some embodiments, the method further comprises returning said eukaryotic cell and/or cells derived therefrom to the subject.

Viral and non-viral based gene transfer methods can be used to introduce nucleic acids encoding components of the present system into cells, tissues, or a subject. Such methods can be used to administer nucleic acids encoding components of the present system to cells in culture, or in a host organism. Non-viral vector delivery systems include DNA plasmids, cosmids, RNA (e.g., a transcript of a vector described herein), a nucleic acid, and a nucleic acid complexed with a delivery vehicle. Viral vector delivery systems include DNA and RNA viruses, which have either episomal or integrated genomes after delivery to the cell. Viral vectors include, for example, retroviral, lentiviral, adenoviral, adeno-associated and herpes simplex viral vectors.

In certain embodiments, plasmids that are non-replicative, or plasmids that can be cured by high temperature may be used, such that any or all of the necessary components of the system may be removed from the cells under certain conditions. For example. this may allow for DNA integration by transforming bacteria of interest, but then being left with engineered strains that have no memory of the plasmids or vectors used for the integration.

Drug selection strategies may be adopted for positively selecting for cells that underwent DNA integration. A donor nucleic acid may contain one or more drug-selectable markers within the cargo. Then presuming that the original donor plasmid is removed, drug selection may be used to enrich for integrated clones. Colony screenings may be used to isolate clonal events.

A variety of viral constructs may be used to deliver the present system (such as one or more Cas proteins, transposon associated proteins, unfoldase proteins (e.g., ClpX), gRNA(s), donor DNA, etc.) to the targeted cells and/or a subject. Nonlimiting examples of such recombinant viruses include recombinant adeno-associated virus (AAV), recombinant adenoviruses, recombinant lentiviruses, recombinant retroviruses, recombinant herpes simplex viruses, recombinant poxviruses, phages, etc. The present disclosure provides vectors capable of integration in the host genome, such as retrovirus or lentivirus. See, e.g., Ausubel et al., Current Protocols in Molecular Biology, John Wiley & Sons, New York, 1989; Kay, M. A., et al., 2001 Nat. Medic. 7(1):33-40; and Walther W. and Stein U., 2000 Drugs, 60(2): 249-71, incorporated herein by reference.

In one embodiment, a DNA segment encoding the present protein(s) is contained in a plasmid vector that allows expression of the protein(s) and subsequent isolation and purification of the protein produced by the recombinant vector. Accordingly, the proteins disclosed herein can be purified following expression, obtained by chemical synthesis, or obtained by recombinant methods.

To construct cells that express the present system, expression vectors for stable or transient expression of the present system may be constructed via conventional methods as described herein and introduced into host cells. For example, nucleic acids encoding the components of the present system may be cloned into a suitable expression vector, such as a plasmid or a viral vector in operable linkage to a suitable promoter. The selection of expression vectors/plasmids/viral vectors should be suitable for integration and replication in eukaryotic cells.

In certain embodiments, vectors of the present disclosure can drive the expression of one or more sequences in prokaryotic cells. Promoters that may be used include T7 RNA polymerase promoters, constitutive E. coli promoters, and promoters that could be broadly recognized by transcriptional machinery in a wide range of bacterial organisms. The system may be used with various bacterial hosts.

In certain embodiments, vectors of the present disclosure can drive the expression of one or more sequences in mammalian cells using a mammalian expression vector. Examples of mammalian expression vectors include pCDM8 (Seed, Nature (1987) 329:840, incorporated herein by reference) and pMT2PC (Kaufman, et al., EMBO J. (1987) 6:187, incorporated herein by reference). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd eds., Cold Spring Harbor Laboratory, Cold Spring Harbor Laboratory Press, Cold Spring Harbor, N.Y., 1989, incorporated herein by reference.

Vectors of the present disclosure can comprise any of a number of promoters known to the art, wherein the promoter is constitutive, regulatable or inducible, cell type specific, tissue-specific, or species specific. In addition to the sequence sufficient to direct transcription, a promoter sequence of the invention can also include sequences of other regulatory elements that are involved in modulating transcription (e.g., enhancers, Kozak sequences and introns). Many promoter/regulatory sequences useful for driving constitutive expression of a gene are available in the art and include, but are not limited to, for example, CMV (cytomegalovirus promoter), EF1a (human elongation factor 1 alpha promoter), SV40 (simian vacuolating virus 40 promoter), PGK (mammalian phosphoglycerate kinase promoter), Ubc (human ubiquitin C promoter), human beta-actin promoter, rodent beta-actin promoter, CBh (chicken beta-actin promoter), CAG (hybrid promoter contains CMV enhancer, chicken beta actin promoter, and rabbit beta-globin splice acceptor), TRE (Tetracycline response element promoter), H1 (human polymerase III RNA promoter), U6 (human U6 small nuclear promoter), and the like. Additional promoters that can be used for expression of the components of the present system, include, without limitation, cytomegalovirus (CMV) intermediate early promoter, a viral LTR such as the Rous sarcoma virus LTR, HIV-LTR, HTLV-1 LTR, Maloney murine leukemia virus (MMLV) LTR, myeoloproliferative sarcoma virus (MPSV) LTR, spleen focus-forming virus (SFFV) LTR, the simian virus 40 (SV40) early promoter, herpes simplex tk virus promoter, elongation factor 1-alpha (EF1-α) promoter with or without the EF1-a intron. Additional promoters include any constitutively active promoter. Alternatively, any regulatable promoter may be used, such that its expression can be modulated within a cell.

Moreover, inducible and tissue specific expression of a RNA, transmembrane proteins, or other proteins can be accomplished by placing the nucleic acid encoding such a molecule under the control of an inducible or tissue specific promoter/regulatory sequence. Examples of tissue specific or inducible promoter/regulatory sequences which are useful for this purpose include, but are not limited to, the rhodopsin promoter, the MMTV LTR inducible promoter, the SV40 late enhancer/promoter, synapsin 1 promoter, ET hepatocyte promoter, GS glutamine synthase promoter and many others. Various commercially available ubiquitous as well as tissue-specific promoters and tumor-specific are available, for example from InvivoGen. In addition, promoters which are well known in the art can be induced in response to inducing agents such as metals, glucocorticoids, tetracycline, hormones, and the like, are also contemplated for use with the invention. Thus, it will be appreciated that the present disclosure includes the use of any promoter/regulatory sequence known in the art that is capable of driving expression of the desired protein operably linked thereto.

The vectors of the present disclosure may direct expression of the nucleic acid in a particular cell type (e.g., tissue-specific regulatory elements are used to express the nucleic acid). Such regulatory elements include promoters that may be tissue specific or cell specific. The term “tissue specific” as it applies to a promoter refers to a promoter that is capable of directing selective expression of a nucleotide sequence of interest to a specific type of tissue (e.g., seeds) in the relative absence of expression of the same nucleotide sequence of interest in a different type of tissue. The term “cell type specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining.

Additionally, the vector may contain, for example, some or all of the following: a selectable marker gene, such as the neomycin gene for selection of stable or transient transfectants in host cells; enhancer/promoter sequences from the immediate early gene of human CMV for high levels of transcription; transcription termination and RNA processing signals from SV40 for mRNA stability; 5′- and 3′-untranslated regions for mRNA stability and translation efficiency from highly-expressed genes like α-globin or β-globin; SV40 polyoma origins of replication and ColE1 for proper episomal replication; internal ribosome binding sites (IRESes), versatile multiple cloning sites; T7 and SP6 RNA promoters for in vitro transcription of sense and antisense RNA; a “suicide switch” or “suicide gene” which when triggered causes cells carrying the vector to die (e.g., HSV thymidine kinase, an inducible caspase such as iCasp9), and reporter gene for assessing expression of the chimeric receptor. Suitable vectors and methods for producing vectors containing transgenes are well known and available in the art. Selectable markers also include chloramphenicol resistance, tetracycline resistance, spectinomycin resistance, streptomycin resistance, erythromycin resistance, rifampicin resistance, bleomycin resistance, thermally adapted kanamycin resistance, gentamycin resistance, hygromycin resistance, trimethoprim resistance, dihydrofolate reductase (DHFR), GPT; the URA3, HIS4, LEU2, and TRP1 genes of S. cerevisiae.

When introduced into the cell, the vectors may be maintained as an autonomously replicating sequence or extrachromosomal element or may be integrated into host DNA.

In one embodiment, the donor DNA may be delivered using the same gene transfer system as used to deliver the Cas protein, and/or transposon associated proteins (included on the same vector) or may be delivered using a different delivery system. In another embodiment, the donor DNA may be delivered using the same transfer system as used to deliver gRNA(s).

In one embodiment, the present disclosure comprises integration of exogenous DNA into the endogenous gene. Alternatively, an exogenous DNA is not integrated into the endogenous gene. The DNA may be packaged into an extrachromosomal or episomal vector (such as AAV vector), which persists in the nucleus in an extrachromosomal state, and offers donor-template delivery and expression without integration into the host genome. Use of extrachromosomal gene vector technologies has been discussed in detail by Wade-Martins R (Methods Mol Biol. 2011; 738:1-17, incorporated herein by reference).

The present system (e.g., proteins, polynucleotides encoding these proteins, donor polynucleotides and compositions comprising the proteins and/or polynucleotides described herein) may be delivered by any suitable means. In certain embodiments, the system is delivered in vivo. In other embodiments, the system is delivered to isolated/cultured cells (e.g., autologous iPS cells) in vitro to provide modified cells useful for in vivo delivery to patients afflicted with a disease or condition.

Vectors according to the present disclosure can be transformed, transfected, or otherwise introduced into a wide variety of cells. Transfection refers to the taking up of a vector by a cell whether or not any coding sequences are in fact expressed. Numerous methods of transfection are known to the ordinarily skilled artisan, for example, lipofectamine, calcium phosphate co-precipitation, electroporation, DEAE-dextran treatment, microinjection, viral infection, and other methods known in the art. Transduction refers to entry of a virus into the cell and expression (e.g., transcription and/or translation) of sequences delivered by the viral vector genome. In the case of a recombinant vector, “transduction” generally refers to entry of the recombinant viral vector into the cell and expression of a nucleic acid of interest delivered by the vector genome.

Any of the vectors comprising a nucleic acid sequence that encodes the components of the present system is also within the scope of the present disclosure. Such a vector may be delivered into host cells by a suitable method. Methods of delivering vectors to cells are well known in the art and may include DNA or RNA electroporation, transfection reagents such as liposomes or nanoparticles to delivery DNA or RNA; delivery of DNA, RNA, or protein by mechanical deformation (see, e.g., Sharei et al. Proc. Natl. Acad. Sci. USA (2013) 110(6): 2082-2087, incorporated herein by reference); or viral transduction. In some embodiments, the vectors are delivered to host cells by viral transduction. Nucleic acids can be delivered as part of a larger construct, such as a plasmid or viral vector, or directly, e.g., by electroporation, lipid vesicles, viral transporters, microinjection, and biolistics (high-speed particle bombardment). Similarly, the construct containing the one or more transgenes can be delivered by any method appropriate for introducing nucleic acids into a cell. In some embodiments, the construct or the nucleic acid encoding the components of the present system is a DNA molecule. In some embodiments, the nucleic acid encoding the components of the present system is a DNA vector and may be electroporated to cells. In some embodiments, the nucleic acid encoding the components of the present system is an RNA molecule, which may be electroporated to cells.

Additionally, delivery vehicles such as nanoparticle- and lipid-based mRNA or protein delivery systems can be used. Further examples of delivery vehicles include lentiviral vectors, ribonucleoprotein (RNP) complexes, lipid-based delivery system, gene gun, hydrodynamic, electroporation or nucleofection microinjection, and biolistics. Various gene delivery methods are discussed in detail by Nayerossadat et al. (Adv Biomed Res. 2012; 1: 27) and Ibraheem et al. (Int J Pharm. 2014 Jan. 1; 459(1-2):70-83), incorporated herein by reference.

### Methods

Also disclosed herein are methods for nucleic acid modification (e.g., insertion/deletion) utilizing the disclosed systems or kits. The methods may comprise contacting a target nucleic acid sequence with a system disclosed herein or a composition comprising the system. The descriptions and embodiments provided above for the engineered CAST system (e.g., the Cas proteins and transposon associated proteins), the at least one unfoldase protein (e.g., ClpX), the gRNA, and the donor nucleic acid are applicable to the methods described herein.

The target nucleic acid sequence may be in a cell. In some embodiments, contacting a target nucleic acid sequence comprises introducing the system into the cell. As described above the system may be introduced into eukaryotic or prokaryotic cells by methods known in the art. In some embodiments, the cell is a mammalian cell. In some embodiments, the cell is a human cell.

In some embodiments, the target nucleic acid is a nucleic acid endogenous to a target cell. In some embodiments, the target nucleic acid is a genomic DNA sequence. The term “genomic,” as used herein, refers to a nucleic acid sequence (e.g., a gene or locus) that is located on a chromosome in a cell.

In some embodiments, the target nucleic acid encodes a gene or gene product. The term “gene product,” as used herein, refers to any biochemical product resulting from expression of a gene. Gene products may be RNA or protein. RNA gene products include non-coding RNA, such as tRNA, rRNA, micro RNA (miRNA), and small interfering RNA (siRNA), and coding RNA, such as messenger RNA (mRNA). In some embodiments, the target nucleic acid sequence encodes a protein or polypeptide.

Polynucleotides containing the target nucleic acid sequence may include, but is not limited to, purified chromosomal DNA, total cDNA, cDNA fractionated according to tissue or expression state (e.g., after heat shock or after cytokine treatment other treatment) or expression time (after any such treatment) or developmental stage, plasmid, cosmid, BAC, YAC, phage library, etc. Polynucleotides containing the target site may include DNA from organisms such as Homo sapiens, Mus domesticus, Mus spretus, Canis domesticus, Bos, Caenorhabditis elegans, Plasmodium falciparum, Plasmodium vivax, Onchocerca volvulus, Brugia malayi, Dirofilaria immitis, Leishmania, Zea maize, Arabidopsis thaliana, Glycine max, Drosophila melanogaster, Saccharomyces cerevisiae, Schizosaccharomyces pombe, Neurospora, Escherichia coli, Salmonella typhimurium, Bacillus subtilis, Neisseria gonorrhoeae, Staphylococcus aureus, Streptococcus pneumonia, Mycobacterium tuberculosis, Aquifex, Thermus aquaticus, Pyrococcus furiosus, Thermus littoralis, Methanobacterium thermoautotrophicum, Sulfolobus caldoaceticus, and others.

The method may comprise administering to the subject, in vivo, or by transplantation of ex vivo treated cells, an effective amount of the described system. In some embodiments, the vector(s) is delivered to the tissue of interest by, for example, an intramuscular, intravenous, transdermal, intranasal, oral, mucosal, or other delivery methods.

The components of the present system or ex vivo treated cells may be administered with a pharmaceutically acceptable carrier or excipient as a pharmaceutical composition. In some embodiments, the components of the present system may be mixed, individually or in any combination, with a pharmaceutically acceptable carrier to form pharmaceutical compositions, which are also within the scope of the present disclosure.

In some embodiments, an effective amount of the components of the present system or compositions as described herein can be administered. As used herein the term “effective amount” may be used interchangeably with the term “therapeutically effective amount” and refers to that quantity that is sufficient to result in a desired activity upon administration to a subject in need thereof. Within the context of the present disclosure, the term “effective amount” refers to that quantity of the components of the system such that successful DNA integration is achieved.

When utilized as a method of treatment, the effective amount may depend on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject. In some embodiments, the subject is a human.

In the context of the present disclosure insofar as it relates to any of the disease conditions recited herein, the terms “treat,” “treatment,” and the like mean to relieve or alleviate at least one symptom associated with such condition, or to slow or reverse the progression of such condition. Within the meaning of the present disclosure, the term “treat” also denotes to arrest, delay the onset (e.g., the period prior to clinical manifestation of a disease) and/or reduce the risk of developing or worsening a disease. For example, in connection with cancer the term “treat” may mean eliminate or reduce a patient's tumor burden, or prevent, delay, or inhibit metastasis, etc.

The phrase “pharmaceutically acceptable,” as used in connection with compositions and/or cells of the present disclosure, refers to molecular entities and other ingredients of such compositions that are physiologically tolerable and do not typically produce untoward reactions when administered to a subject (e.g., a mammal, a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. “Acceptable” means that the carrier is compatible with the active ingredient of the composition (e.g., the nucleic acids, vectors, cells, or therapeutic antibodies) and does not negatively affect the subject to which the composition(s) are administered. Any of the pharmaceutical compositions and/or cells to be used in the present methods can comprise pharmaceutically acceptable carriers, excipients, or stabilizers in the form of lyophilized formations or aqueous solutions.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g., Remington: The Science and Practice of Pharmacy 20th Ed. (2000) Lippincott Williams and Wilkins, Ed. K. E. Hoover.

The methods may be used for a variety of purposes. For example, the methods may include, but are not limited to, inactivation of a microbial gene, RNA-guided DNA integration in a plant or animal cell, methods of treating a subject suffering from a disease or disorder (e.g., cancer, Duchenne muscular dystrophy (DMD), sickle cell disease (SCD), β-thalassemia, and hereditary tyrosinemia type I (HT1)), and methods of treating a diseased cell (e.g., a cell deficient in a gene which causes cancer).

### Kits

Also within the scope of the present disclosure are kits that include the components of the present system.

The kit may include instructions for use in any of the methods described herein. The instructions can comprise a description of administration of the present system or composition to a subject to achieve the intended effect. The instructions generally include information as to dosage, dosing schedule, and route of administration for the intended treatment. The kit may further comprise a description of selecting a subject suitable for treatment based on identifying whether the subject is in need of the treatment.

The kits provided herein are in suitable packaging. Suitable packaging includes, but is not limited to, vials, bottles, jars, flexible packaging, and the like.

The packaging may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

The kit may further comprise a device for holding or administering the present system or composition. The device may include an infusion device, an intravenous solution bag, a hypodermic needle, a vial, and/or a syringe.

The present disclosure also provides for kits for performing DNA integration in vitro. The kit may include the components of the present system. Optional components of the kit include one or more of the following: buffer constituents, control plasmid, sequencing primers, cells, and the like.

### EXAMPLES

The following are examples of the present invention and are not to be construed as limiting.

The following nomenclature details are applicable: Tn6677 encodes a naturally occurring Cas8-Cas5 fusion protein, as part of the Type I-F CRISPR-Cas system, referred to herein as Cas8, for simplicity; the Type I-F CRISPR-Cas system encoded within Tn7-like transposons may be more specifically referred to as Type I-F3, however Type I-F may be used for simplicity; the complex known as TniQ-Cascade, or QCascade (for simplicity), comprises crRNA (one copy), Cas8 (one copy), Cas7 (six copies), Cas6 (one copy), and TniQ (two copies); in some contexts, QCascade subunits have been referred to with other gene and protein naming schemes, e.g. Csy1 or Csy2 or Cas8f instead of Cas8; Csy3 or Cas7f Cas7; Csy4 or Cas6f instead of Cas6; the mini-transposon, also known as a mini-Tn, refers to the mobilizable DNA containing a cargo/payload sequence flanked by conserved left (L) and right (R) ends of the transposon; the mini-Tn may be encoded within a larger donor DNA molecule, for example a plasmid-based donor, or pDonor. Guide RNA (gRNA) for CRISPR-associated transposon (CAST) systems may be equivalently referred to as CRISPR RNA (crRNA), and herein gRNA and crRNA are used synonymously. Finally, CAST systems may also be referred to as INTEGRATE systems; CRISPR-transposon systems; CRISPR-Tn systems; RNA-guided transposase systems; RNA-guided DNA integration system; or a similar set of synonymous terms to refer to the core technology as molecular machinery. RNA-guided DNA integration by CAST systems may involve a diverse array of targeting proteins, which include Cascade from Type I-B, Type I-D, and Type I-F CRISPR-Cas systems, and Cas12k from Type V-K CRISPR-Cas systems.

### Materials and Methods

Plasmid construction. Genes were human codon-optimized and synthesized by Genscript, and plasmids were generated using a combination of restriction digestion, ligation, Gibson assembly, and inverted (around-the-horn) PCR. All PCR fragments for cloning were generated using Q5 DNA Polymerase (NEB).

The CRISPR array sequence (repeat-spacer-repeat) for VchCAST is as follows: 5′-GTGAACTGCCGAGTAGGTAGCTGATAAC (SEQ ID NO: 172)-N₃₂-GTGAACTGCCGAGTAGGTAGCTGATAAC (SEQ ID NO: 172)-3′ where N₃₂ represents the 32-nt guide region.

The sequence of the mature crRNA is as follows: 5′-CUGAUAAC (SEQ ID NO: 173)-N₃₂-GUGAACUGCCGAGUAGGUAG (SEQ ID NO: 174)-3′.

The CRISPR array sequence (repeat-spacer-repeat) for PseCAST is as follows: 5′-GTGACCTGCCGTATAGGCAGCTGAAAAT (SEQ ID NO: 175)-N₃₂-GTGACCTGCCGTATAGGCAGCTGAAAAT (SEQ ID NO: 175)-3′ where N32 represents the 32-nt guide region.

The sequence of the mature crRNA is as follows: 5′-CUGAAAAU (SEQ ID NO: 176)-N₃₂-GUGACCUGCCGUAUAGGCAG (SEQ ID NO: 177)-3′.

‘Atypical’ repeats (See, Klompe, S. E. et al. Mol. Cell 82, 616-628.e5 (2022) and Petassi, M. T., Hsieh, S. & Peters, J. E. Cell 183, 1757-1771.e18 (2020), incorporated herein by reference) were also used for PseCAST (unless otherwise mentioned) to reduce the likelihood of recombination during cloning. For these variant CRISPR arrays, the repeat-spacer-repeat sequence is as follows: 5′-GTGACCTGCCGTATAGGCAGCTGAAGAT (SEQ ID NO: 178)-N₃₂-TAATTCTGCCGAAAAGGCAGTGAGTAGT (SEQ ID NO: 179)-3′ where N32 represents the N₃₂-nt guide region. The sequence of the mature crRNA is as follows: 5′-CUGAAGAU (SEQ ID NO: 180)-N₃₂-UAAUUCUGCCGAAAAGGCAG (SEQ ID NO: 181)-3′. Where noted, the 32-nt guide region was modified to have varying lengths. The repeat sequences flanking the guide region were not modified in these experiments.

Clp proteins from the E. coli genome were PCR amplified from BL21 DE3 cells with primers that specifically amplified the open reading frame of the indicated protein and cloned into pcDNA3.1 expression vectors with an N-terminal bipartite-NLS tag. ClpX sequences from E. coli, Pseudoalteromonas sp., and V. cholerae were then codon-optimized by Genscript and ordered as Twist fragments to be cloned into pcDNA3.1 expression vectors with an N-terminal bipartite-NLS tag.

E. coli culturing and general transposition assays. Chemically competent E. coli BL21 (DE3) cells carrying pDonor, pDonor and pTnsABC, or pDonor and pQCascade, were prepared and transformed with 150-250 ng of pEffector, pQCascade, or pTnsABC, respectively. Transformations were plated on agar plates with the appropriate antibiotics (100 μg/ml spectinomycin, 100 μg/ml carbenicillin, 50 μg/ml kanamycin) and 0.1 mM IPTG. For bacterial transposition assays investigating PseCAST activity, cells were co-transformed with pEffector and pDonor. Cells were incubated for 18-20 h at 37° C. and typically grew as densely spaced colonies, before being scraped, resuspended in LB medium, and prepared for subsequent analysis. A full list of all plasmids used for transposition experiments is provided in Table 1, and a list of crRNAs used is provided in Table 3.

E. coli qPCR analysis of transposition products. The optical density of resuspended colonies from the transposition assays was measured at 600 nm, and approximately 3.2×10⁸ cells (the equivalent of 200 μl of OD600=2.0) were pelleted by centrifugation at 4,000×g for 5 min. The cell pellets were resuspended in 80 μl of H₂O, before being lysed by incubating at 95° C. for 10 min in a thermal cycler. The cell debris was pelleted by centrifugation at 4,000×g for 5 min, and 5 μl of lysate supernatant was removed and serially diluted in water to generate 20- and 500-fold lysate dilutions for qPCR analysis.

Integration in the T-RL orientation was measured by qPCR by comparing Cq values of a T-RL-specific primer pair (one transposon- and one genome-specific primer) to a genome-specific primer pair that amplifies an E. coli reference gene (rssA). Transposition efficiency was then calculated as 2^{ΔCq}, in which ΔCq is the Cq difference between the experimental reaction and the reference reaction. qPCR reactions (10 μl) contained 5 μl of SsoAdvanced Universal SYBR Green Supermix (BioRad), 1 μl H2O, 2 μl of 2.5 μM primers, and 2 μl of 500-fold diluted cell lysate. Reactions were prepared in 384-well white PCR plates (BioRad), and measurements were performed on a CFX384 Real-Time PCR Detection System (BioRad) using the following thermal cycling parameters: polymerase activation and DNA denaturation (98° C. for 3 min), and 35 cycles of amplification (98° C. for 10 s, 59° C. for 1 min).

Mammalian cell culture and transfections. HEK293T cells were cultured at 37° C. and 5% CO₂. Cells were maintained in DMEM media with 10% FBS and 100 U/mL of penicillin and streptomycin (Fisher Scientific). The cell line was authenticated by the supplier and tested negative for mycoplasma.

Cells were typically seeded at approximately 100,000 cells per well in a 24-well plate (Eppendorf or Fisher Scientific) coated with poly-D-lysine (Fisher Scientific), 24 hours prior to transfection. Cells were transfected with DNA mixtures and 2 μl of Lipofectamine 2000 (Fisher Scientific), per the manufacturer's instructions. Transfection reactions typically contained between 1 μg and 1.5 μg of total DNA. For detailed transfection parameters specific to distinct assays, please refer to the sections below.

Western immunoblotting and nuclear/cytoplasmic fractionation. Cells were transfected with epitope-tagged protein expression plasmids. Approximately 72 hours after transfection, cells were washed with PBS and harvested using Cell Lysis Buffer (150 mM NaCl, 0.1% Triton X-100, 50 mM Tris-HCl pH 8.0, Protease inhibitor (Sigma Aldrich)). For nuclear and cytoplasmic fractionation experiments, cells were harvested using Cell Lysis Buffer (Thermo Fisher Scientific) per the manufacturer's instructions. Proteins were separated by SDS-PAGE and transferred to a PVDF membrane (Fisher Scientific). The membrane was then washed with TBS-T (50 mM Tris-Cl, pH 7.5, 150 mM NaCl, 0.1% Tween-20) and blocked with blocking buffer (TBS-T with 5% w/v BSA). Membranes were then incubated with primary antibodies overnight at 4° C. in blocking buffer. Membranes were then washed and incubated with secondary antibodies at room temperature for one hour. All antibodies (both primary and secondary) were diluted 1:10,000 in blocking buffer. Membranes were again washed and then developed with SuperSignal West Dura (Thermo Fisher).

HEK293T fluorescent reporter assays and flow cytometry analysis and sorting. HEK293T cells were seeded at approximately 50,000 cells per well in a 24-well plate coated with poly-D-lysine 24 hours prior to transfection. For Cas6-mediated RNA processing assays, cells were co-transfected with 300 ng of GFP-reporter plasmid, 300 ng of pCas6, and 10 ng of an mCherry expression plasmid (as a transfection marker). In negative control experiments, cells were transfected with 300 ng of a pdCas9 instead of a pCas6 to control for possible expression burden or squelching. For transcriptional activation assays, cells were co-transfected with 60 ng of reporter plasmid, 20 ng of a plasmid encoding an orthogonal fluorescent protein (as a transfection marker), and the additional indicated plasmids. In separate wells, cells were transfected with 100 ng of Cas9-based transcriptional activators and 50 ng of either a non-targeting or targeting sgRNA as positive controls.

DNA mixtures were transfected using 2 μl of Lipofectamine 2000 (Fisher Scientific), per the manufacturer's instructions. Approximately 72-96 hours after transfection, cells were collected for assay by flow cytometry. Transfected cells were analyzed by gating based on fluorescent intensity of the transfection marker relative to a negative control (see Yeo, N. C. et al. Nat. Methods 15, 611-616 (2018)). For assays that involved cell sorting, cells were transfected with a GFP expression plasmid and collected 4 days after transfection. A BD FACS Aria flow cytometer was used to sort cells and obtain flow cytometry data. Cells with the top 20% brightest GFP fluorescence were sorted by 5% increments into 4 bins. Cells were immediately harvested after sorting, as detailed below.

HEK293T genomic activation and RT-qPCR analysis. HEK293T cells were seeded at approximately 50,000 cells per well in a 24-well plate coated with poly-D-lysine 24 hours prior to transfection. Cells were co-transfected as described above, with the following VchCAST components: 100 ng pTnsAB_{f}, 50 ng pTnsC-VP64, 50 ng pTniQ, 50 ng pCas6, 250 ng pCas7, 50 ng pCas8, and 62.5 ng each of 4 targeting crRNAs for TIN, MIAT, and ASCL1 (or 83.3 ng each of 3 targeting crRNAs for ACTC1) (pCRISPR). In control experiments, cells were co-transfected with 100 ng of either pdCas9-VP64 or pdCas9-VPR plasmid, 62.5 ng each of 4 targeting sgRNAs for TTN (psgRNA), and a pUC19 plasmid to standardize transfected DNA amounts. Cells were harvested 72 hours after transfection using the RNeasy Plus Mini Kit (Qiagen), according to the manufacturer's instructions. cDNA was subsequently synthesized using the iScript cDNA Synthesis Kit (BioRad) using 1000 ng of RNA in a 20 uL reaction. Gene-specific qPCR primers were designed to amplify an approximately 180-250 bp fragment to quantify the RNA expression of each gene, and a separate pair of primers was designed to amplify ACTB (beta-actin) reference gene for normalization purposes.

qPCR reactions (10 μl) contained 5 μl of SsoAdvanced Universal SYBR Green Supermix (BioRad), 2 μl H₂O, 1 μl of 5 μM primer pair, and 2 μl of cDNA diluted 1:4 in H₂O. Reactions were prepared in 384-well white PCR plates (BioRad), and measurements were performed on a CFX384 Real-Time PCR Detection System (BioRad) using the following thermal cycling parameters: polymerase activation and DNA denaturation (98° C. for 2 min), 40 cycles of amplification (95° C. for 10 s, 60° C. for 30 s), and terminal melt-curve analysis (65-95° C. in 0.5° C. per 5 s increments). Each condition was analyzed using three biological replicates, and two technical replicates were run per sample. Normalized gene activation was calculated as the ratio of the 2-ΔCq of the targeting samples to the non-targeting samples, in which ΔCq is the Cq difference between the experimental gene primer pair and the reference gene primer pair.

Chromatin Immunoprecipitation. For ChIP-seq analysis experiments, HEK293T cells were seeded at approximately 1,500,000 cells per well in a 10 cm dish coated with poly-D-lysine 24 hours prior to transfection. Cells were co-transfected as described above with the following eCAST-1 components: 1.5 μg p3× FLAG-TnsC, 1.5 μg pTniQ, 1.5 μg pCas6, 7.5 μg pCas7, 1.5 μg pCas8, and 3 μg of either a targeting (TTN crRNA 1) or non-targeting crRNA. 72 hours after transfection, cells were harvested and pelleted by centrifugation at 300×g for 5 minutes, and the supernatant was aspirated. In brief, pellets were resuspended in 1% freshly made formaldehyde (Thermo Fisher Scientific in DPBS and shaken gently for 10 minutes. Fixation was quenched by adding 2.5 M glycine, for a final concentration of 125 mM glycine, and rotating cells for 5 minutes. Cells were pelleted, washed with cold DPBS, pelleted, resuspended in DPBS and 1× cOmplete EDTA free protease inhibitors (Sigma Aldrich), pelleted, flash frozen in liquid nitrogen, and stored at −80° C.

On the day of sonication, the cross-linked pellets were resuspended in 1 mL of Lysis Buffer 1 (50 mM HEPES-KOH, 140 mM NaCl, 1 mM EDTA, 10% glycerol, 0.5% NP-40, 0.25% Triton X-100) and 1× protease inhibitors and rotated for 10 minutes. Cells were pelleted at 1350 g for 5 minutes. Pellets were resuspended in 1 mL of Lysis Buffer 2 (10 mM Tris-HCl, 200 mM NaCl, 1 mM EDTA, 0.5 mM EGTA) and 1× protease inhibitors and rotated for 10 minutes before being pelleted at 1350 g for 5 minutes. Pellets were resuspended in 900 uL of Lysis Buffer 3 (10 mM Tris-HCl, 100 mM NaCl, 1 mM EDTA, 0.5 mM EGTA, 0.1% Na-Deoxycholate, 0.5% N-lauroylsarcosine), 100 uL of 10% Triton X-100, and 1× protease inhibitors. All steps took place at 4° C.

The resuspended cells were transferred to 1 ml milliTUBE AFA Fiber (Covaris) and sonicated on M220 Focused-ultrasonicator (Covaris) under the following SonoLab 7.2 settings: minimum temperature 4° C., set point 6° C., maximum temperature 7° C., Peak Power 75.0, Duty Factor 10.0, Cycles/Burst 200, sonication time 490 seconds. Sonicated cell lysate was centrifuged at 20,000 g for 10 minutes at 4° C. The supernatant was transferred to a new tube, and 5% was saved as the input sample. The remaining supernatant was incubated with Dynabeads Protein G (Thermo Fisher Scientific) that were bound to the monoclonal anti-Flag M2 antibody at a 1:8 dilution (Sigma-Aldrich) the day before sonication by overnight rotating at 4° C., and the lysate-Dynabead mixture was rotated overnight at 4° C.

The samples were washed three times each with low salt buffer (150 mM NaCl, 0.1% SDS, 1% Triton X-100, 1 mM EDTA, 50 mM Tris HCl), high salt buffer (550 mM NaCl, 0.1% SDS, 1% Triton X-100, 1 mM EDTA, 50 mM Tris HCl), and LiCl buffer (150 mM LiCl, 0.5% Na-deoxycholate, 0.1% SDS, 1% Nonidet P-40, 1 mM EDTA, 50 mM Tris HCl) on a magnetic stand at 4° C. The samples were washed with 1 mL of TE buffer (1 mM EDTA, 10 mM Tris HCl) with 50 mM NaCl and centrifuged at 960 g for 3 minutes at 4° C. The supernatant was aspirated and 210 μL of elution buffer (1% SDS, 50 mM Tris HCl, 10 mM EDTA, 200 mM NaCl) was added to samples and incubated for 30 minutes at 65° C. Samples were centrifuged for 1 minute at 16,000 g at room temperature, and 200 μL of supernatant was incubated overnight at 65° C. The input sample was diluted in 150 μL of elution buffer and also incubated overnight at 65° C. 0.5 μL of 10 mg/mL RNase was added, and samples were incubated for 1 hour at 37° C. 2 μL of 20 mg/mL Proteinase K were added, and samples were incubated for 1 hour at 55° C. The DNA was recovered by the QiaQUICK PCR Purification Kit (Qiagen) and DNA was eluted in 50 μL of water for downstream analysis.

ChIP-seq Sample Preparation. Sample DNA concentration was determined by the DeNovix dsDNA High Sensitivity Kit. Illumina libraries were generated using the NEBNext Ultra II Dna Library Prep Kit for Illumina (NEB). Sample concentrations were normalized such that 12 ng of DNA in each condition was used for library preparation. The concentration of DNA was determined for pooling using the DeNovix dsDNA High Sensitivity Kit. Illumina libraries were sequenced in paired-end mode on the Illumina NextSeq platforms with automated demultiplexing and adaptor trimming. For each ChIP-seq sample, 75-bp paired end reads were obtained and between 9.5 and 18.9 million uniquely mapped fragments were analyzed.

ChIP-seq analysis. ChIP-seq data were processed using CoBRA v2.0 with modifications as follows. Each experimental condition (TnsC with TTN-targeting gRNA or TnsC with non-targeting [NT] gRNA) was processed with three biological replicate ChIP samples and one corresponding non-immunoprecipitated input sample. Reads were aligned to the hg38 human reference genome using BWA-MEM with default settings. Reads were sorted and indexed using SAMtools, and multi-mapping reads with a MAPQ score<1 were removed using the samtools view command. Peaks were called using MACS2 v2.2.6. The callpeak function was executed in paired-end mode with the following parameters: −g 2.7e9 −q 0.0001—keep-dup auto—nomodel. Input samples were used as controls for peak calling. Bedgraph files for each sample with pileup information in signal per million reads (SPMR) were generated with the—SPMR and −B subcommands of MACS2 callpeak and were converted to bigwig files using bedGraphToBigWig. ChIP-seq signal at individual genomic loci was visualized with IGV. Reads mapping to the Y chromosome or the mitochondrial genome were removed prior to downstream analysis.

A consensus list of peaks for each experimental condition was identified using bedtools v2.30.0. First, peak files for the three replicates were concatenated and sorted and overlapping peaks were merged. Then, peaks appearing in fewer than three replicates were removed. Blacklisted regions of the genome defined by the ENCODE Consortium were also removed. The consensus lists for the conditions were then intersected to identify peaks exclusive to either condition (bedtools intersect −v) or peaks shared by both conditions (bedtools intersect −u). Differential binding analysis was performed using DiffBind v3.6.5 to compare ChIP-seq read density between the two conditions in the regions defined by their consensus peak lists. Reads were counted using dba.count with the following arguments: summits=F, bUseSummarizeOverlaps=T, bRemoveDuplicates=F, bSubControl=F. Read counts were normalized to account for differences in sequencing depth between samples. Normalized read counts were passed to DESeq2 to calculate the mean across conditions, as well as fold change and q-value (using the Benjamini-Hochberg procedure) between conditions, for each peak. The result of differential binding analysis was visualized using ggplot2.

Heatmaps of ChIP-seq signal intensity over peaks exclusive to the TTN gRNA condition were plotted using deepTools v3.3.2. Score matrices were generated using computeMatrix in reference-point mode. Peaks were sorted in descending order by mean signal over 2 kb windows around peak centers before plotting using plotHeatmap.

For manual inspection of potential off-target sites, a custom script was used to identify genomic loci with high similarity to the TTN spacer sequence. Other than the TTN locus itself, no loci with fewer than 5 mismatches were identified. TnsC ChIP-seq signal at the 5 most similar loci was visualized with IGV.

HEK293T integration assays. For assays in which plasmids were isolated and used to transform bacteria, HEK293T cells were transfected with requisite eCAST-1 expression plasmids, a pDonor that contained a non-replicative origin of replication (R6K), a pTarget plasmid, and a crRNA expression plasmid (pCRISPR) that either encoded a non-targeting crRNA or a crRNA targeting pTarget. 72 hours after transfection, cells were washed with PBS, harvested using TrypLE (Fisher Scientific), neutralized with culture media, and pelleted. After removal of supernatant, transfected plasmids were harvested using Qiagen Miniprep columns per the manufacturer's instructions, and further concentrated using the Qiagen MinElute column. Of this final purified plasmid mixture, 1 μl was used to electroporate NEB 10-beta electrocompetent E. coli cells (NEB) per the manufacturer's instructions. After recovery at 37° C., cells were plated onto LB-agar plates containing chloramphenicol. Chloramphenicol-resistant colonies were then replated onto LB-agar plates containing both chloramphenicol and kanamycin, and doubly-resistant colonies were harvested for genotypic analyses.

For all other integration assays, HEK293T cells were counted using a Countess 3 Cell Counter and seeded at 20,000 cells per well, unless otherwise specified, in a 24-well plate coated with poly-D-lysine 24 hours prior to transfection. Cells were transfected using plasmid DNA mixtures and 2 μl of Lipofectamine 2000, per the manufacturer's instructions. For eCAST-1 transposition assays, HEK293T cells were transfected with the following optimized VchCAST components, unless otherwise stated: 300 ng of pTnsAB_{f}, 25 ng of pTnsC, 100 ng each of pTniQ, pCas6, pCas7, pCas8, 200 ng of pDonor, 100 ng pTarget, and 100 ng of a targeting or non-targeting crRNA (pCRISPR). For eCAST-2 transposition assays, HEK293T cells were transfected with the following PseCAST components, unless otherwise specified: 200 ng of pTnsAB_{f}, 50 ng each of pTnsC, pTniQ, pCas6, pCas7, and pCas8, 200 ng of pDonor, and 100 ng of pTarget and a targeting or non-targeting crRNA (pCRISPR). When a QCascade polycistronic expression vector was used (pQCas), 75 ng was transfected. For eCAST-3 transposition assays, eCAST-2 conditions were used with pQCas, and 20 ng of pClpX was co-transfected as well (unless otherwise noted). All eCAST-3 transposition assays utilized puromycin selection (unless otherwise noted, see below for puromycin conditions), as constitutive ClpX expression led to visible toxicity independent of CAST machineries. Unless otherwise stated, cells were cultured for 4 days after transfection. Cells were washed with DPBS with no calcium or magnesium (Fisher Scientific), harvested using TrypLE (Fisher Scientific), and neutralized with culture media. 20% of the resuspended cells were pelleted by centrifugation at 300×g for 5 minutes, and the supernatant was aspirated. Cell pellets were resuspended in 50 μL of Quick Extract (Lucigen), and genomic DNA was prepared per the manufacturer's instructions.

For assays that utilized puromycin selection, HEK293T cells were transfected as described above with the addition of 20 ng of puromycin resistance expression plasmid as a transfection marker. Media was changed 24 hours after transfection, and selection with 1 μg/mL of puromycin was started. Cells were harvested using Quick Extract (Lucigen) per the manufacturer's instructions, either 4 days after transfection, or for timecourse experiments, beginning at 2 days after transfection until 6 days after transfection, with or without puromycin selection. For plasmid-based assays that utilized cell sorting, HEK293T cells were transfected with eCAST-2 components as described above with an additional 5 ng of GFP expression plasmid as a transfection marker. 4 days after transfection, the GFP positive cells with the brightest mean fluorescence intensity were sorted in 4 bins of 5% increments to encompass the 20% brightest cells and were immediately harvested as described above. For genomic assays that utilized cell sorting, HEK293T cells were seeded at approximately 100,000 cells in 6 well plates coated with poly-D lysine 24 hours before transfection. Cells were transfected with the following eCAST-3 components: 1000 ng each of pTnsAB_{f} and pDonor, 250 ng of pTnsC, 375 ng of polycistronic pCas7-Cas8-Cas6-TniQ, 20 ng of pGFP, 100 ng of pClpX, and 500 ng of a targeting crRNA (pCRISPR). 4 days after transfection, the top 20% of GFP positive cells with the brightest mean fluorescence intensity were sorted and immediately harvested, as described above. For genomic integration assays, cells were harvested by previously described assays, using 100 μl of freshly prepared lysis buffer (10 mM Tris-HCl, pH 7.5; 0.05% SDS; 25 μg/ml proteinase K (ThermoFisher Scientific) directly into each well of the tissue culture plate. The genomic DNA mixture was incubated at 37° C. for 1-2 h, followed by an 80° C. enzyme inactivation step for 30 min.

For assays that utilized cargo sizes ranging from 798 bp to 15 kb, HEK293T cells were transfected as described above with eCAST-2 component plasmids, except the 5 kb, 10 kb, and 15 kb pDonor plasmids were transfected in molar equivalents to the 798 bp pDonor (˜406 fmol), to account for the size difference between donor plasmids. For assays that utilized amplicon deep sequencing, HEK293T cells were transfected as described above, with a pDonor plasmid that contained a primer binding site immediately downstream of the right transposon end that matched a primer binding site present in the unedited pTarget plasmid. Cells were harvested 4days after transfection.

Nested PCR analysis of transposition assays. DNA amplification was performed by PCR using Q5 Hot Start High-Fidelity DNA Polymerase (NEB) following the manufacturer's protocol. In brief, PCR-1 1 μL of cell lysate was added to a 25 μL PCR reaction. Thermocycling conditions were as follows: 98° C. for 45 seconds, 98° C. for 15 seconds, 66° C. for 15 seconds, 72° C. for 10 seconds, 72° C. for 2 minutes, with steps 2-4 repeated 24 times. The annealing temperature was adjusted depending on primers used. 1 μL of the first PCR reaction served as the template for PCR-2, a 25 μL PCR reaction that was run under the same thermocycling conditions. Primer pairs contained one target-specific primer and one transposon-specific primer, and the primers used in the second PCR reaction generated a smaller amplicon than the first reaction. PCR amplicons were resolved by 1-2% agarose gel electrophoresis and visualized by staining with SYBR Safe (Thermo Scientific). Negative control samples were always analyzed in parallel with experimental samples to identify mis-priming products, some of which presumably result from the analysis being performed on crude cell lysates that still contain the pDonor and target-site DNA.

qPCR Analysis of Plasmid-to-Plasmid and Genomic Integration Products.
Transposition-specific qPCR primers were designed to amplify a ˜140-bp fragment to quantify integration efficiency. Primer pairs were designed to span the integration junction, with the forward primer annealing to pTarget, or the genome, and the reverse primer annealing within the transposon. Additionally, a custom 5′ FAM-labeled, ZEN/3′ IBFQ probe (IDT) was designed to anneal to each unique integration junction. A separate pair of primers and a SUN-labeled, ZEN/3′ IBFQ probe (IDT) were designed to amplify a distinct reference sequence in the target plasmid or the human genome, for efficiency calculation purposes.

Probe-based qPCR reactions (10 μL) contained 5 μL of TaqMan Fast Advanced Master Mix, 0.5 μL of each 18 μM primer pair, 0.5 μL of each 5 μM probe, 1 μL of H₂O, and 2 μL of ten-fold diluted cell lysate for plasmid-based transposition samples, or 2 μL of five-fold diluted cell lysate for genomic transposition samples. Reactions were prepared in 384-well white PCR plates (BioRad), and measurements were performed on a CFX384 Real-Time PCR Detection System (BioRad) using the following thermal cycling parameters: polymerase activation (95° C. for 10 minutes) and 50 cycles of amplification (95° C. for 15 seconds, 59.5° C. for 1 minute). Each condition was analyzed using either two or three biological replicates, and two technical replicates were run per sample. Baseline threshold ratios were manually adjusted to be 1:1 for the reference primer pair to the transposition primer pair. Integration efficiency was calculated as a percentage as 2^{-ΔCq} times 100, in which ΔCq is the Cq difference between the reference primer pair and the transposition primer pair.

To analyze the frequency of left-right insertion (T-LR) versus right-left insertion (T-RL) of the PseCAST transposon in plasmid-based assays, integration-specific qPCR primers were designed to span the T-LR integration junction, in addition to the primer pairs used for T-RL integration and the reference amplicon in the probe-based qPCR analysis described above. qPCR reactions (10 μL) contained 5 μl of SsoAdvanced Universal SYBR Green Supermix (BioRad), 2 μl H₂O, 1 μl of 5 μM primer pair, and 2 μl of ten-fold diluted cell lysate. Reactions were prepared in 384-well white PCR plates (BioRad), and measurements were performed on a CFX384 Real-Time PCR Detection System (BioRad) using the following thermal cycling parameters: polymerase activation and DNA denaturation (98° C. for 2 min), 50 cycles of amplification (95° C. for 10 s, 59.5° C. for 20 s), and terminal melt-curve analysis (65-95° C. in 0.5° C. per 5 s increments). Each condition was analyzed using three biological replicates, and two technical replicates were run per sample.

ddPCR analysis of integration products. During harvesting of HEK293T plasmid-based integration assays, 50% of the resuspended cells were reserved during lysate generation. 500 μL of resuspended cells were pelleted by centrifugation at 300×g for 5 minutes. The supernatant was aspirated, and DNA was extracted from cell pellets using the Qiagen DNeasy Blood and Tissue Kit (Qiagen). DNA was eluted in H₂O and diluted to a concentration of 2.5 ng/μL. For genomic integration assays, crude cell lysate, generated as described above, was purified using two-sided AMPure XP beads (Beckman Coulter) as follows: 45 μL of AMPure XP beads were added to 20-80 μL of genomic lysate and incubated for 5 minutes before being placed on a magnetic PCR rack for 5 minutes. The supernatant was aspirated, and the beads were washed twice with 80% ethanol. The beads were dried for 5 minutes, then 25 μL of water was added to resuspend the beads. The suspension was incubated for 10 minutes off the magnetic rack, then placed back on the rack for 5 minutes. The supernatant was transferred to a new tube.

ddPCR was performed with the same primers and probes as for plasmid-to-plasmid integration analysis and genomic integration assays with the exception of the OXA1L-2 target site, which was not quantified via qPCR. Plasmid-based ddPCR reactions (20 μL) contained 10 μL of ddPCR Supermix for Probes (Biorad), 1 μL of each 5 μM probe, 1 μL of each 18 μM primer pair, 5 units of HindIII (NEB), 4.13 μL of H₂O, and 2 μL of 2.5 ng/μL DNA. Genomic ddPCR reactions (20 μL) contained 10 μL of ddPCR Supermix for Probes (Biorad), 1 μL of each 5 μM probe, 1 μL of each 18 μM primer pair, 5 units of HindIII (NEB), and 6.33 μL of purified DNA, ranging from ˜6 ng to ˜500 ng. Reactions were assembled at room temperature, and droplets were generated using the Biorad QX200 Droplet Generator according to the manufacturer's instructions. Thermocycling was performed on a Biorad C1000 Touch Thermocycler with the following parameters: enzyme activation (95° C. for 10 minutes), 40 cycles of amplification (94° C. for 30 second, 61.5° C. for 1 minute) and enzyme deactivation (98° C. for 10 minutes). After thermocycling, droplets were hardened at 4° C. for 2 hours. Droplets were analyzed using the QX200 Droplet Reader according to the manufacturer instructions. Integration percentages were calculated as the number of FAM positive molecules divided by the number of SUN/VIC positive molecules times 100.

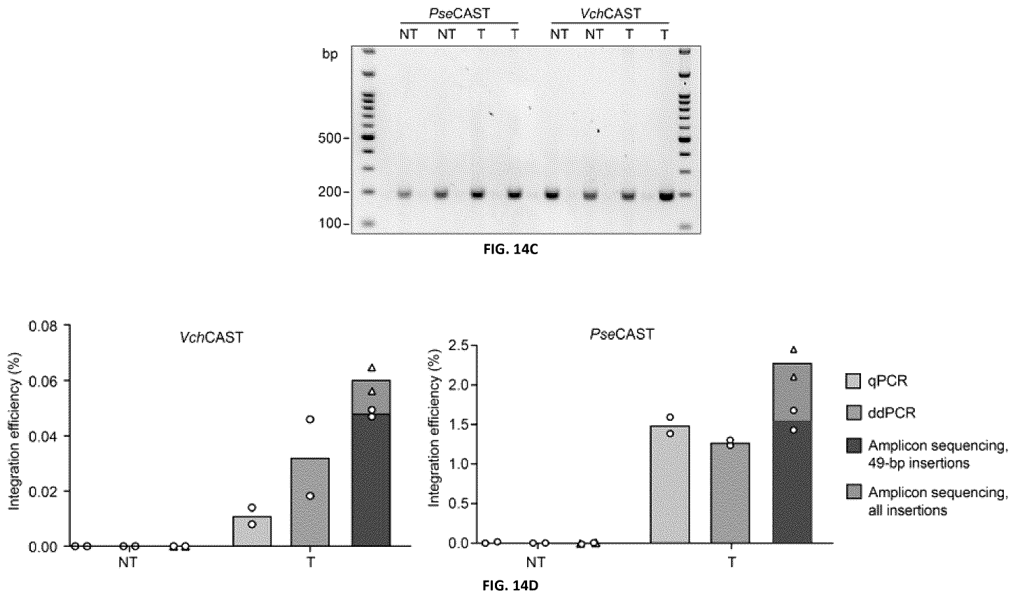

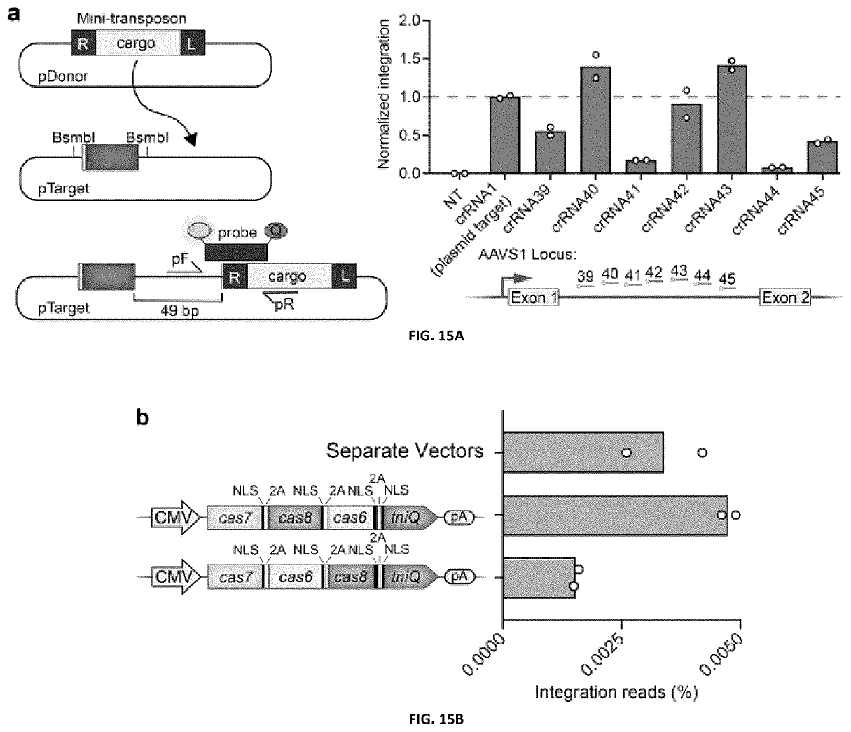

Amplicon sequencing strategy to quantify integration efficiencies. To improve sensitivity of genomic integration assays in human cells, an NGS-based approach was designed in which both unedited sites and integration products are simultaneously amplified in a single PCR reaction (FIG. 14B). PCR-1 products were generated as described for PCR-1 in the nested PCR analyses, except primers contained universal Illumina adaptors as 5′ overhangs and the cycle number was reduced to 15 for plasmid-to-plasmid integration assays, and 25 for genomic integration assays. Additionally, up to 5 degenerate nucleotides were placed between the primer binding site and the Illumina adaptor 5′ overhang to improve library diversity when sequencing. 1 μl of lysate per 10 μl of overall PCR reaction was used; plasmid-to-plasmid integration assays were 20 μl PCR reactions, while genomic integration assays were 250 μl PCR reactions to sample sufficient alleles. These products were then diluted 20-fold into a fresh polymerase chain reaction (PCR-2) containing indexed p5/p7 primers and subjected to 10 additional thermal cycles using an annealing temperature of 65° C. After verifying amplification by analytical gel electrophoresis, barcoded reactions were pooled and resolved by 2% agarose gel electrophoresis, DNA was isolated by Gel Extraction Kit (Qiagen), and NGS libraries were quantified by qPCR using the NEBNext Library Quant Kit (NEB). Illumina sequencing was performed using the NextSeq platform with automated demultiplexing and adaptor trimming (Illumina).

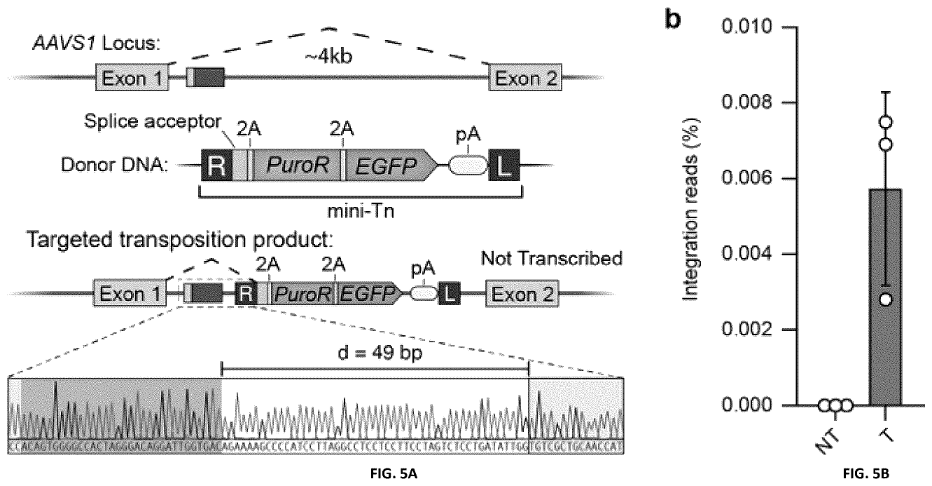

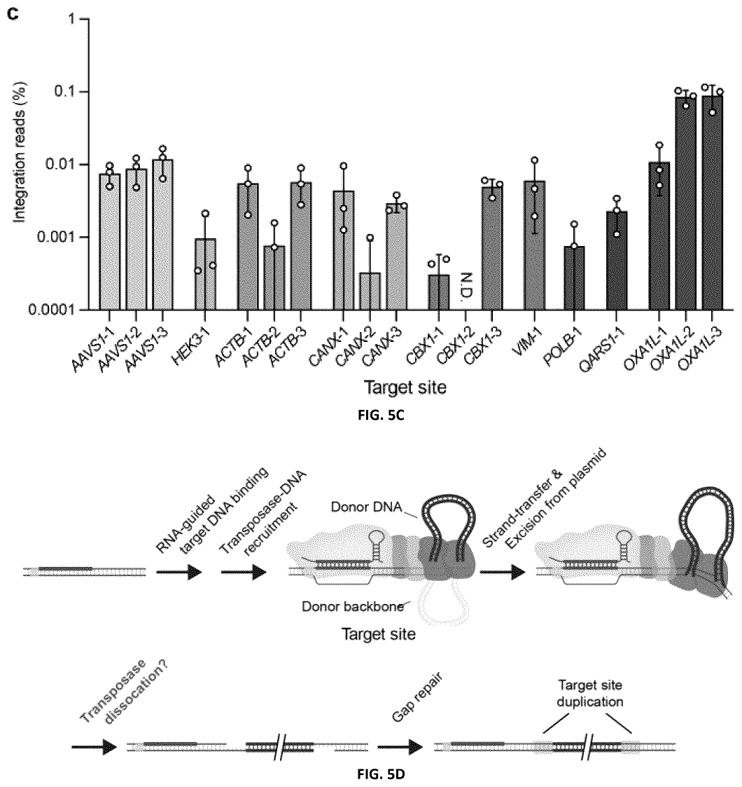

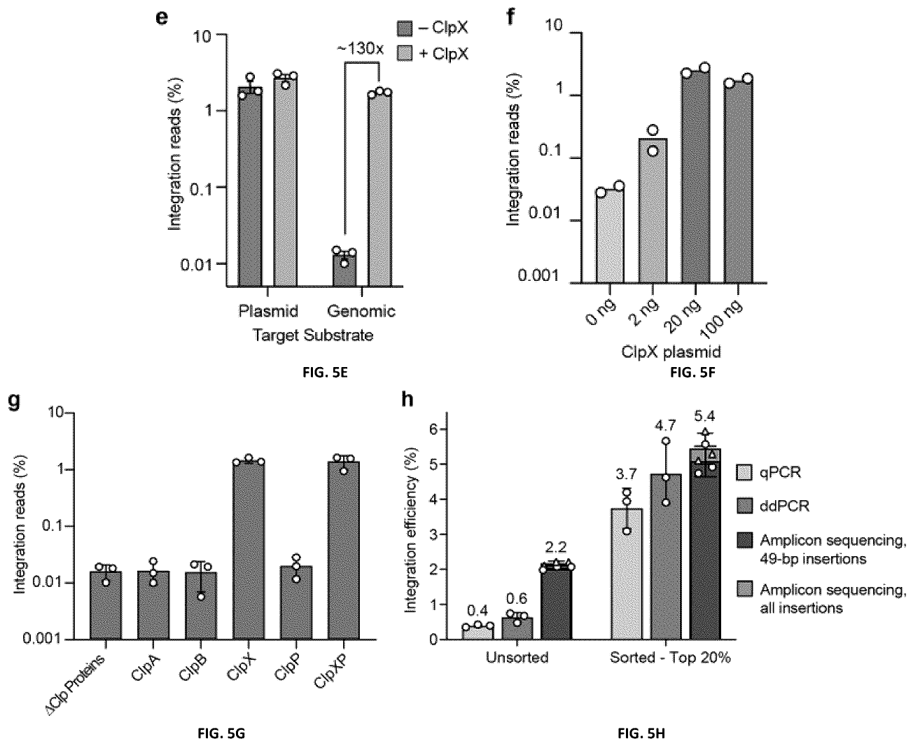

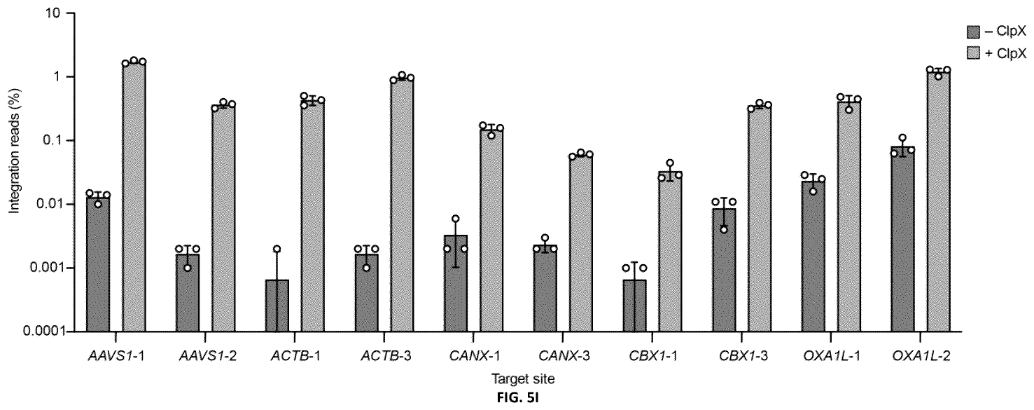

To determine integration efficiencies and distributions, reads were filtered that contained the expected 10-bp sequence immediately downstream of the forward primer, to verify that they derived from the target site. Next, reads containing a 10-bp transposon end sequence were counted as “integration reads,” and the integration distance was calculated from the start of the transposon end to the PAM-distal end of the target sequence. Reads that instead contained a 10-bp sequence from the unedited site at the end of the read were counted as “unedited reads.” The integration efficiency, or “integration reads (%)”, as marked in FIGS. 5B-5G, was calculated as the number of “integration reads” divided by the sum of both “integration reads” and “unedited reads”, converted to a percentage. Histograms of integration distances were plotted by compiling distances across all reads within a given sample.

Data availability. Sequencing data has been deposited in the National Center for Biotechnology Information Sequence Read Archive under GEO accession GSE223174.

### Example 1

### Identification of a Bacterial Factor Involved in RNA-Guided DNA Integration

Previously, a diverse set of CAST systems that encode nuclease-deficient type I-F CRISPR-Cas systems were identified and shown to catalyze RNA-guided DNA integration into extra-chromosomal (e.g., plasmid) DNA targets in human cells at varying efficiencies. A specific CAST system derived from Tn7016 in Pseudoalteromonas sp. S983, referred to as PseCAST, exhibited RNA-guided DNA integration at plasmid target sites at efficiencies ranging from roughly 0.5-5%, whereas the efficiencies for RNA-guided DNA integration at genomic target sites ranged from 0.01% to 0.1%, as shown in FIG. 19A. This discrepancy between efficiencies observed at plasmid versus genomic targets could be explained by a number of possible factors, including, but not limited to, the copy number of the target site, the chromatin state (e.g., whether the DNA is occluded by nucleosomes or not), the topology of the DNA (e.g., supercoiling), the cellular localization of the substrate, and the sequence complexity of the DNA substrate, among other possible explanations, as outlined in FIG. 19B.

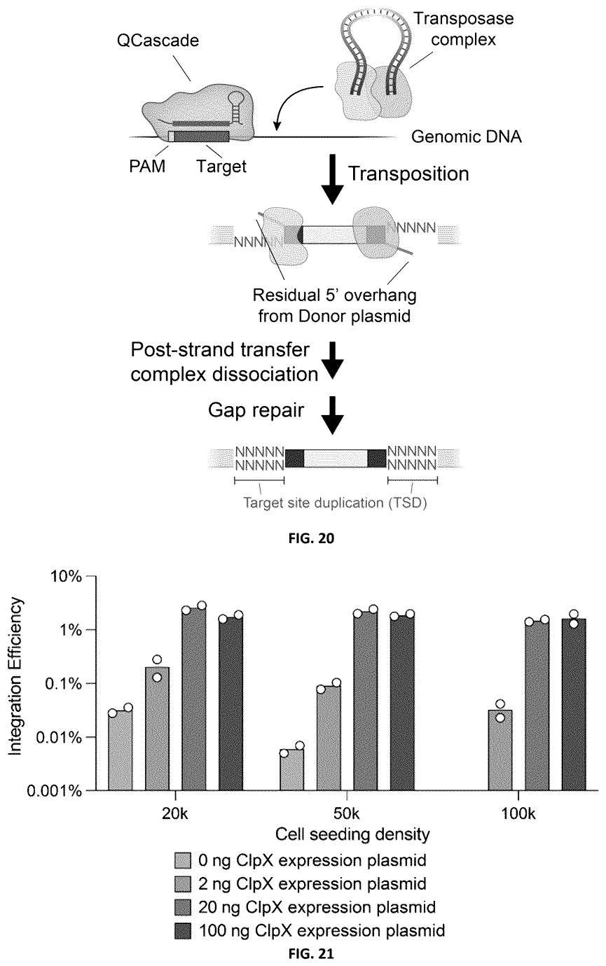

Another potential difference could involve the extent to which integration product intermediates are recognized and acted upon by endogenous DNA repair proteins, to complete the entire editing reaction and generate resolved integration products (FIG. 20). Tn7-like CAST systems, specifically those that also encode a TnsA endonuclease protein, catalyze cut-and-paste transposition that leaves DNA double-strand breaks behind on the donor DNA molecule after excision, and generates gapped intermediate products at the target site after the strand-transfer reaction, which covalently joins the 3′-hydroxyl ends of the excised (mini)-transposon DNA substrate with the target DNA at a 5-bp staggered site. Excision of the (mini)-transposon DNA from the donor DNA molecule requires enzymatic activity of both TnsA (endonuclease) and TnsB (DDE-family transposase), whereas the strand-transfer reaction requires only the TnsB proteins. Importantly, two monomers must both catalyze reactions concurrently to join both ends of the inserted DNA with the target site. The initial intermediate products then contain 5-nt gaps on both sides of the inserted DNA, which must be filled in by a DNA polymerase enzyme, followed by a ligation reaction, to complete the overall DNA integration (e.g., transposition) pathway. This pathway ultimately yields simple-insertion DNA products, which are characterized by hallmark 5-bp target-site duplications (TSDs) that are a consequence of the 5-nt gap fill-in reaction that occurs on both ends of the inserted DNA. Importantly, gap fill-in requires disassembly of the post-strand transfer transpososome complex, in order to render the DNA accessible to DNA polymerase and ligase for completion of the terminal reaction steps.

pcDNA3.1-derivated plasmids that encode an NLS-tagged ClpX protein, which was subcloned from the genome of E. coli BL21 (DE3) strain, were generated to enable robust expression and nuclear localization of EcoClpX in human cells (DNA and protein sequences can be found in Tables 1 and 2). HEK293T cells were co-transfected with ClpX expression plasmids, along with all required machinery for PseCAST to carry out RNA-guided DNA integration. crRNAs targeting either plasmid or genomic target sites for RNA-guided DNA integration were expressed, and integration activity was quantified using a next-generation sequencing (NGS)-based approach, in which unedited and edited (DNA-inserted) alleles are amplified using the same set of primers, due to the presence of a genomic primer binding site within the mini-transposon cargo. An approximate 100× increase in integration efficiencies was observed at genomic target sites in the presence of EcoClpX, whereas integration efficiencies at ectopic plasmid target sites exhibited little change with the addition of ClpX (FIG. 5E).

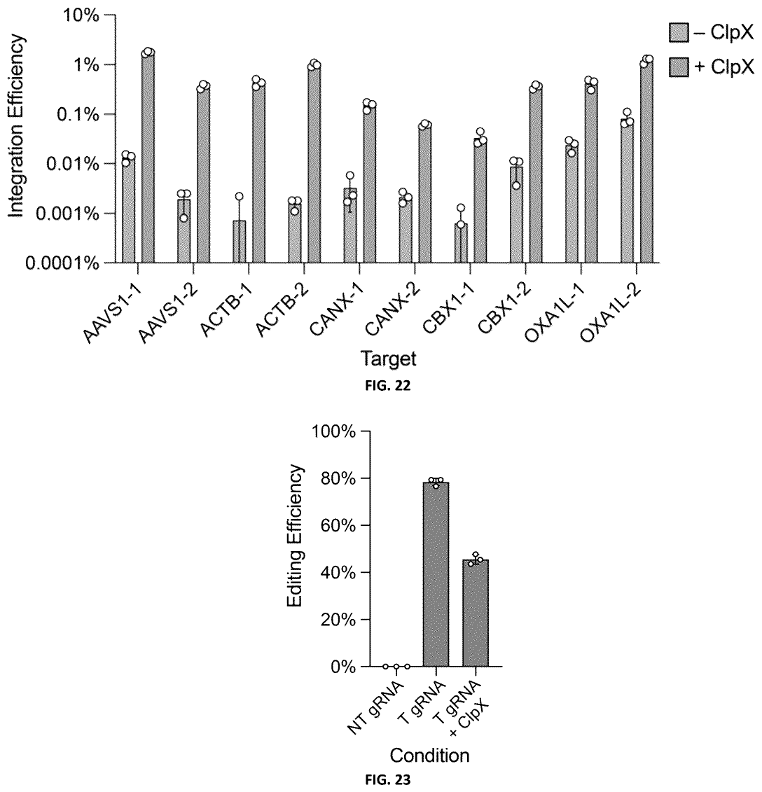

The impact of ClpX on CAST-mediated DNA integration into genomic target sites was investigated by titrating the amount of ClpX expressed in the host cell. The amount of ClpX-expression plasmid was serially increased from 0 ng to 100 ng, as shown in FIG. 21, and the seeding density of cells was modulated approximately 20-24 hours prior to transfection. A dose response was observed in the editing efficiency at genomic target sites as a function of ClpX expression plasmid amount, where integration efficiency increased as more plasmid was transfected, until the effect was saturated at 100 ng. The ability for ClpX to increase genomic integration efficiencies was investigated by targeting multiple loci across the genome, and comparing integration efficiencies in the presence and absence of ClpX. As shown in FIG. 22, ClpX universally improved genomic integration efficiencies; this increase was between approximately 10- and 600-fold.

ClpX is part of a large multi-protein degradation pathway in bacteria, which also involves other proteins including ClpA, ClpB, and ClpP. ClpP is a large, tetradecameric subunit peptidase, which has no intrinsic protein specificity. ClpP can form a proteolytic complex with either ClpA or ClpX. ClpA recognizes substrates with abnormal N-termini sequences, while ClpX recognizes C-termini motifs, such as the SsrA sequence. ClpB has approximately 80% sequence identity to ClpA, but is an AAA+ ATPase chaperone that functions independent of ClpP. In order to determine whether ClpX was specifically involved in enhancing RNA-guided DNA integration at genomic target sites in mammalian cells, or if other members of this multi-protein degradation pathway could also similarly act as accessory factors, similar experiments as those described above were performed, but ClpX was substituted with either ClpA, ClpB, ClpP, or a combination of both ClpX and ClpP simultaneously. As shown in FIG. 5G, genomic integration efficiencies were only improved in the presence of ClpX, but not ClpA or ClpB, and the additional presence of ClpP did not further impact genomic integration efficiencies. These results indicate that the unfoldase activity of ClpX, but not the proteolytic activity of ClpP, is sufficient to enhance the integration efficiency of CAST systems in mammalian cells. This enhancement may be due to the specific unfolding and active disassembly of post-transposition complexes, thereby rendering the DNA integration intermediate product accessible to enzymes for gap fill-in and ligation and may indicate the presence of protein-protein interactions between ClpX and one or more components of CAST systems present in the post-strand transfer (e.g., post-transposition) complex.

The described above demonstrated an enhancement of RNA-guided DNA integration at genomic target sites using ClpX derived from the E. coli BL21(DE3) genome. However, CAST systems referred to here as PseCAST and VchCAST are derived from species that are not within the Escherichia genus, and derive instead from a Pseudoalteromonas genus and Vibrio cholerae, respectively. In some embodiments, the native ClpX from the species matched with the particular CAST system is instead used to enhance RNA-guided DNA integration activity, such that the ClpX derives from a cellular environment where it may have co-evolved more closely with the components from the CAST system. Human codon-optimized (hCO) DNA fragments were cloned that encode ClpX proteins from both Pseudoalteromonas sp. and Vibrio cholerae into pcDNA3.1-like vectors, and they were co-transfected with PseCAST and VchCAST, respectively. As shown in FIG. 16B, PseCAST and VchCAST exhibited enhanced genomic integration efficiencies with both E. coli derived ClpX (EcoClpX) and with their respective native ClpX proteins (PseClpX and VchClpX, respectively). Notably, VchCAST exhibited near-undetectable integration efficiencies in the absence of ClpX altogether, whereas in the presence of ClpX, integration efficiencies were enhanced to detectable levels of ˜0.01%.

EcoClpX was tested in combination with a more conventional gene editing system, namely SpyCas9 together with a sgRNA, in order to determine whether the enhancement effect of ClpX is specific to CAST, or whether there is some more general, non-specific enhancement activity. When the AAVS1 locus within intron 1 of the PPP1R12C gene was targeted for standard gene editing with CRISPR-Cas9, using amplicon-sequencing to determine editing efficiencies as measured by the frequency of indel reads compared to wild-type (unedited) reads, EcoClpX failed to enhance the observed editing efficiencies for CRISPR-Cas9 (FIG. 23). Rather, there was a minor ˜2× decrease in editing efficiency, possibly due to squelching effects or impacts on cellular fitness as a consequence of ClpX expression.

### Example 2

### Investigating TnsB-ClpX Interactions Via Plasmid and Genomic Integration Assays

The specific interactions between ClpX and component(s) of Type I-F CAST systems are unknown. PseCAST is active for targeted integration at both episomal plasmid DNA and genomic DNA sites in the absence of ClpX protein, and the addition of ClpX selectively enhances integration efficiency at genomic target sites, but not plasmid DNA sites.

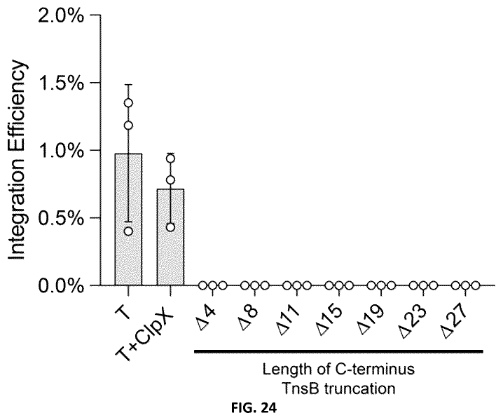

A panel of sequence truncations of PseTnsB beginning from the C-terminus was generated and the efficiency of integration into plasmid target sites for each truncation mutant was tested. As shown in FIG. 24, introducing even a 4-aa truncation at the C-terminus of TnsB resulted in a complete loss of plasmid-based integration activity, suggesting that these residues are involved in some aspect of the integration reaction that is independent of ClpX.

### Example 3

### Heterologous Expression of CAST Components in Human Cells

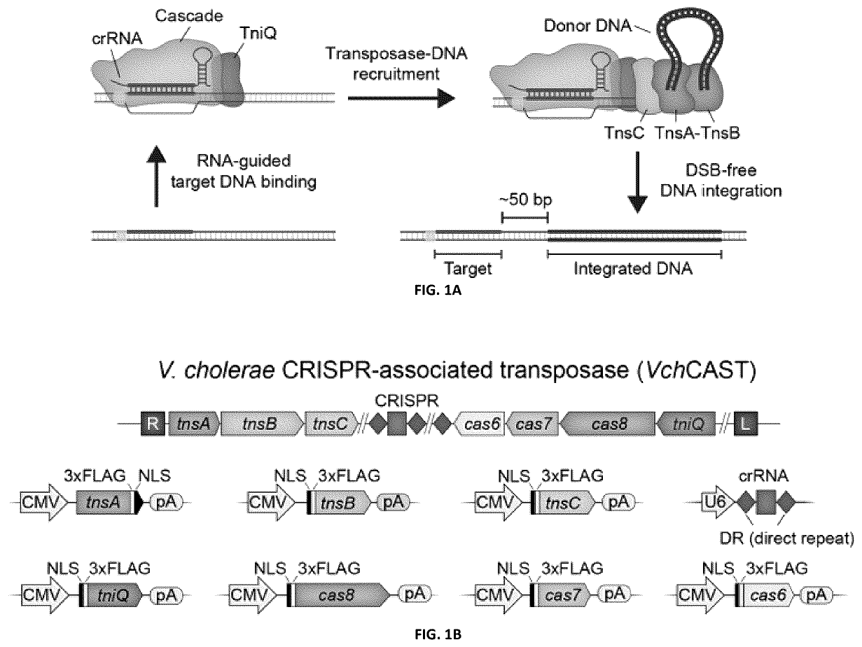

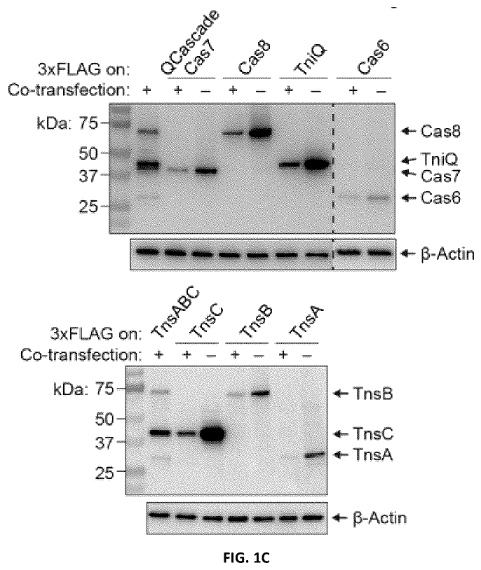

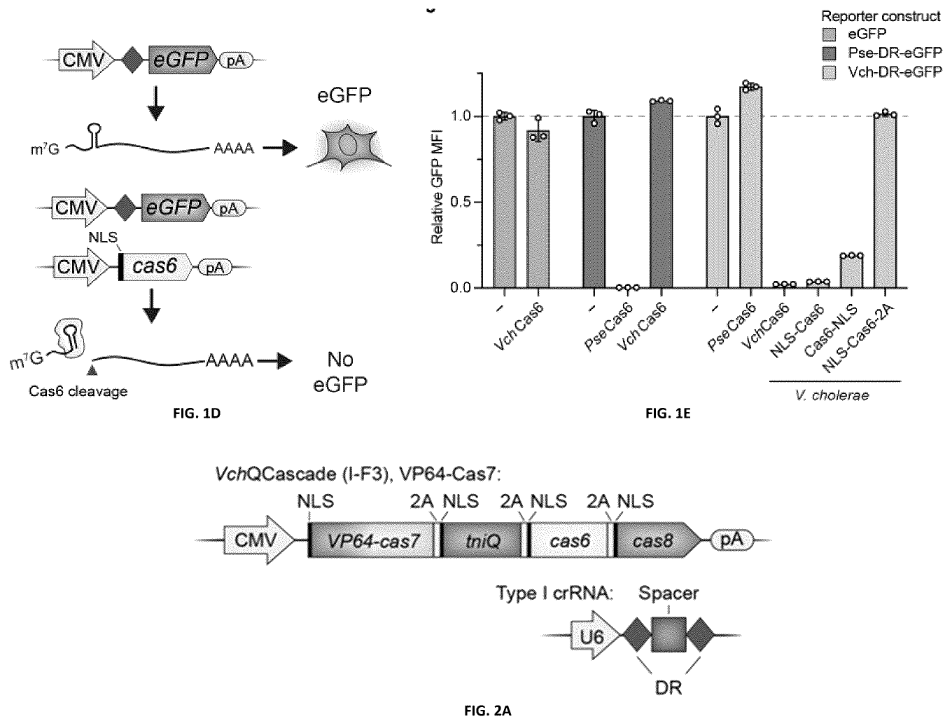

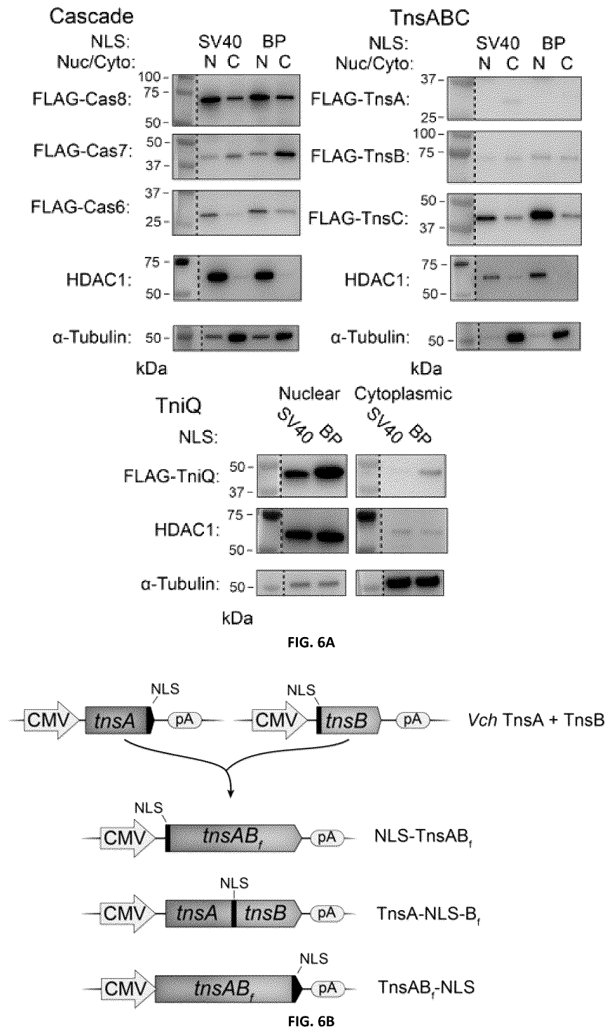

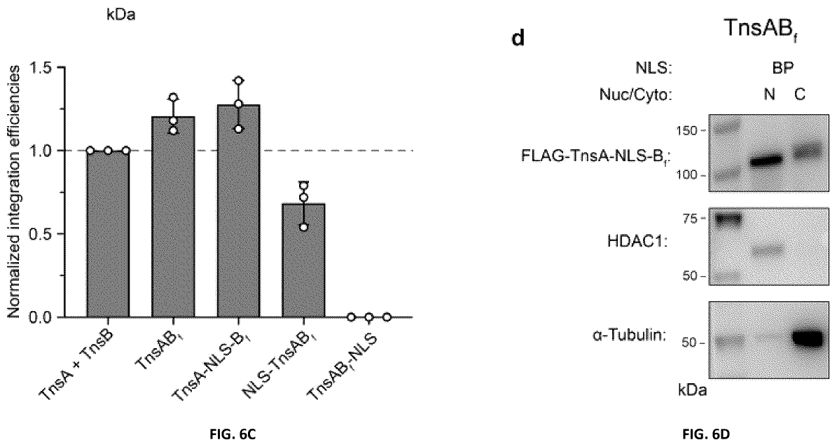

To establish whether the component parts of Type I-F V. cholerae CAST (VchCAST; previously also referred to as VchINTEGRATE) were efficiently expressed, each protein-coding gene was cloned onto a standard mammalian expression vector with an N- or C-terminal nuclear localization signal (NLS) and 3× FLAG epitope tag (FIG. 1B). Using Western blotting, robust heterologous protein expression was shown both individually and when all CAST proteins were co-expressed (FIG. 1C). Cellular fractionation provided evidence of nuclear trafficking, and efficient expression and trafficking of an engineered TnsAB fusion protein (TnsAB_{f}) that retains wild-type activity was also demonstrated (FIG. 6). However, initial attempts to reconstitute RNA-guided DNA integration in HEK293T cells proved unsuccessful, even after exploring numerous strategies to enrich rare events through both positive and negative selection. To separately assess guide RNA expression a previously developed approach (See, Chen, Y. et al. Nat. Commun. 11, 1-4 (2020)). was adapted to monitor crRNA biogenesis within the 5′ untranslated region (UTR) of a GFP-encoding mRNA. Cas6 is a ribonuclease subunit of Cascade that cleaves the CRISPR repeat sequence in most Type I CRISPR-Cas systems, which would sever the 5′ cap from the GFP open reading frame and thus lead to fluorescence knockdown (FIG. 1D). Accordingly, a near-total loss of GFP fluorescence was observed when the reporter plasmid was co-transfected with cognate VchCas6, but not when the reporter encoded a non-cognate CRISPR repeat or lacked a repeat altogether (FIG. 1E). Interestingly, GFP knockdown was substantially reduced when Cas6 contained a C-terminal NLS or 2A peptide (FIG. 1E), indicating a sensitivity to terminal tagging that could not be explained by the cryoEM structure (see below).

### Example 4

### QCascade and TnsC Function as Transcriptional Activators

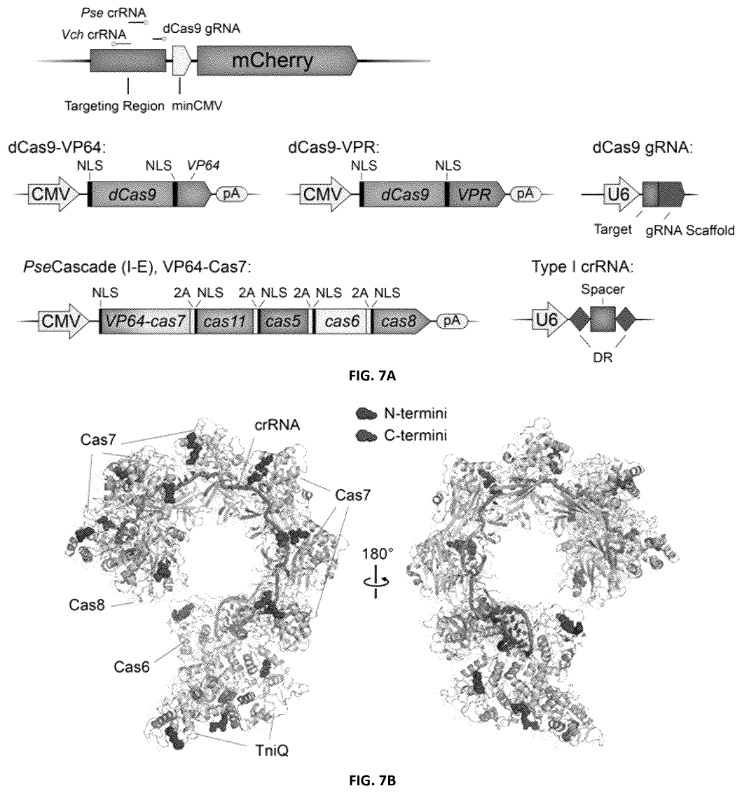

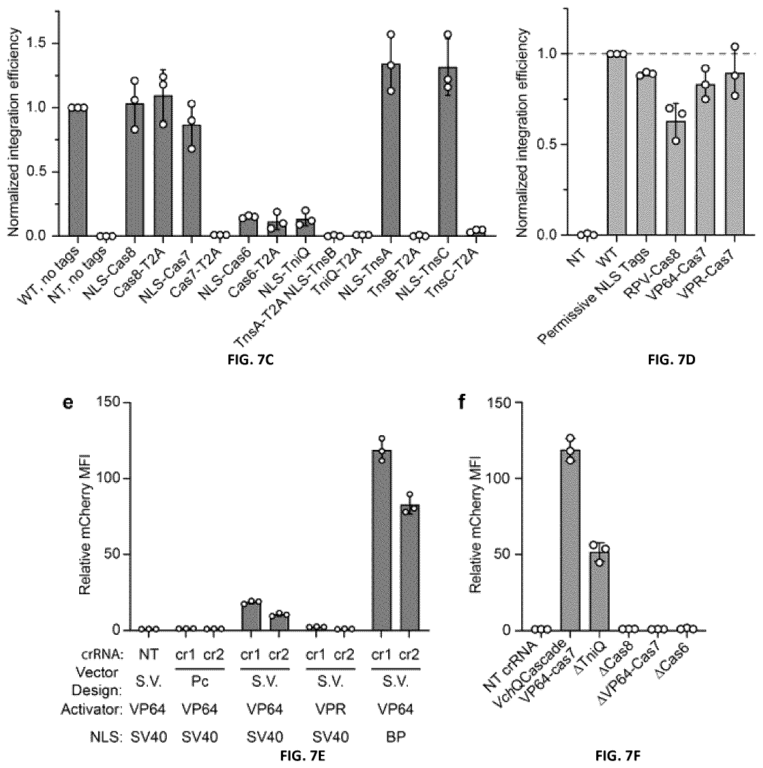

Unlike most Type II and V CRISPR-Cas systems, which encode single-effector proteins that function as RNA-guided DNA nucleases (Cas9 and Cas12, respectively), the Cascade complex encoded by Type I systems does not possess DNA cleavage activity and instead exhibits long-lived target DNA binding upon R-loop formation, analogously to catalytically inactive Cas9 (dCas9). This activity was leveraged for transcriptional activation of an mCherry reporter gene by fusing transcriptional activators to QCascade, thereby converting DNA binding into a detectable signal that would allow facile troubleshooting and optimization of QCascade function (FIG. 7A).

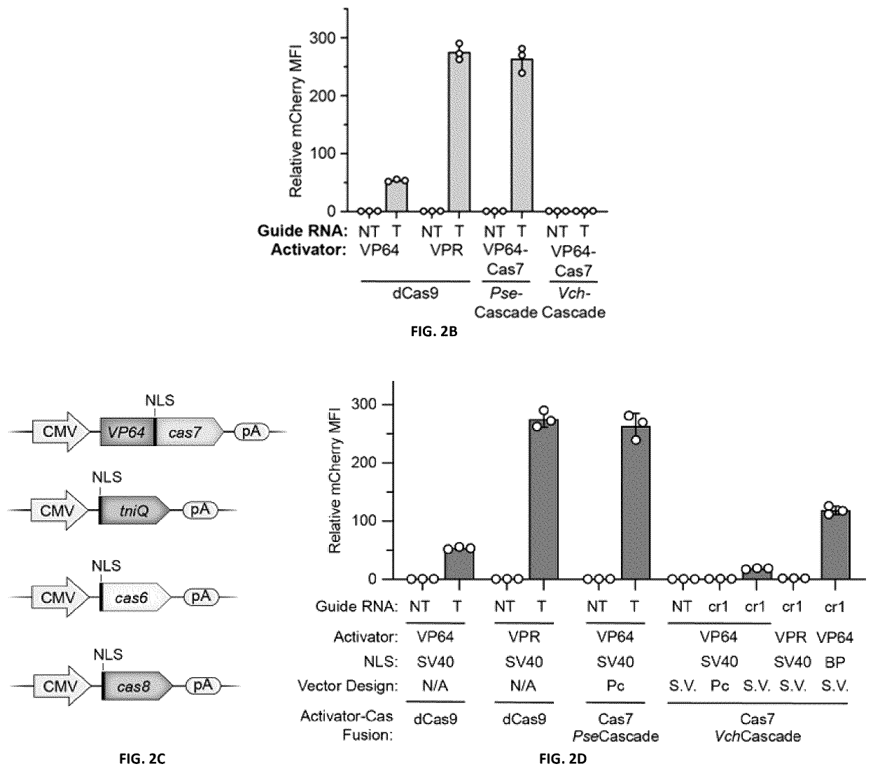

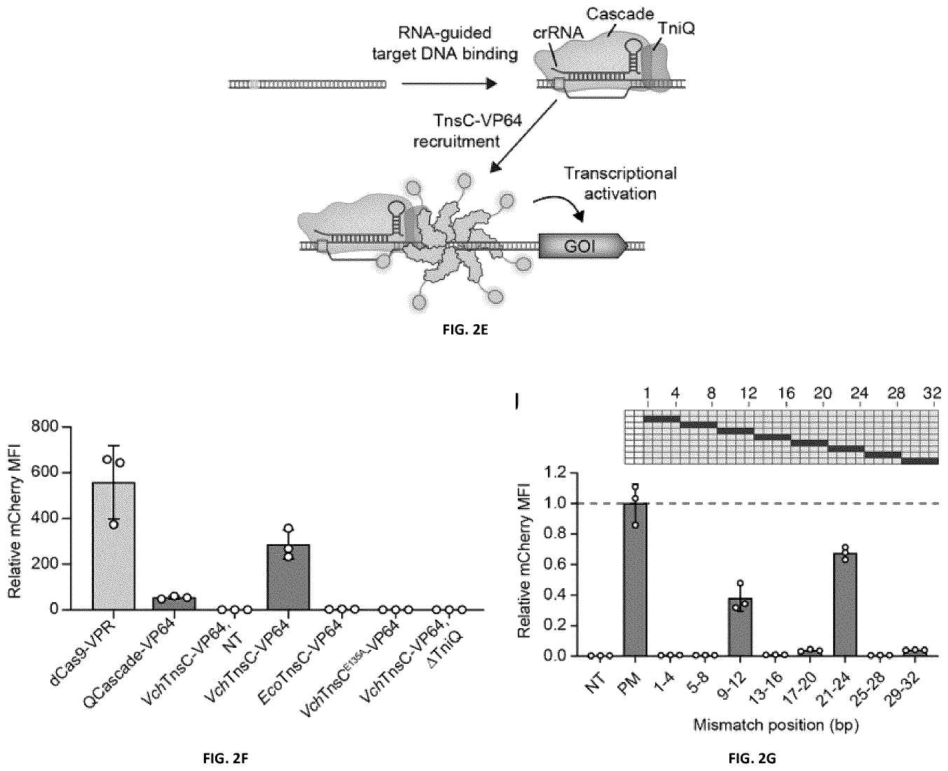

Activators using a Type I-E Cascade unrelated to transposons from Pseudomonas sp. S-6-2 (PseCascade_IE) were constructed. VP64 was fused to the hexameric Cas7 subunit and all five cas genes were concatenated within a single polycistronic vector downstream of a CMV promoter, by linking them together with virally derived 2A ‘skipping’ peptides; the crRNA was separately expressed from a U6 promoter (FIG. 7A). The resulting expression plasmids yielded ˜260-fold mCherry activation when co-transfected with the reporter plasmid, similar to levels achieved with dCas9-VPR, and the effect was ablated in the presence of a non-targeting crRNA (FIG. 2B). Surprisingly, nearly identical designs using the transposon-encoded Type I-F QCascade homolog from V. cholerae, failed to result in detectable activation (FIGS. 2A-2B).

To systematically investigate if presence of N-terminal NLS tags, C-terminal 2A tags, or both, might be inhibiting QCascade assembly and/or RNA-guided DNA targeting, peptide tags were cloned onto the termini of all VchCAST components and their impact was tested in E. coli transposition assays. While some tags had little effect on activity, others led to a severe reduction or complete loss of targeted DNA integration (FIG. 7C). The transposase components were particularly vulnerable, with an N-terminal tag on TnsA and C-terminal tags on TnsB and TnsC being largely prohibitive. Within the context of QCascade, C-terminal 2A tags on TniQ and Cas7 each reduced integration by >90%, which could explain the lack of transcriptional activation observed using polycistronic vector designs. Multiple components were screened for activator fusions and the N-terminus of Cas7 was amenable to both VP64 and VPR fusions in bacteria (FIG. 7D).

QCascade-VP64 was tested in human cells using individual expression vectors with optimized NLS tag locations for each component, and mCherry activation was detected for two distinct crRNAs, evidencing successful assembly and target binding in human cells (FIGS. 2C, 2D and 7E). Activation levels were further increased by replacing all monopartite SV40 NLS tags with bipartite (BP) NLS tags, and this activity was dependent on the simultaneous expression of Cas8, Cas7, Cas6, and a targeting crRNA (FIGS. 2D, 7E-7F). Interestingly, although Cas7 tolerated a VPR fusion in bacteria, transcriptional activation was unable to be detected in mammalian cells using VPR-Cas7 (FIGS. 2D, 7D-7E).

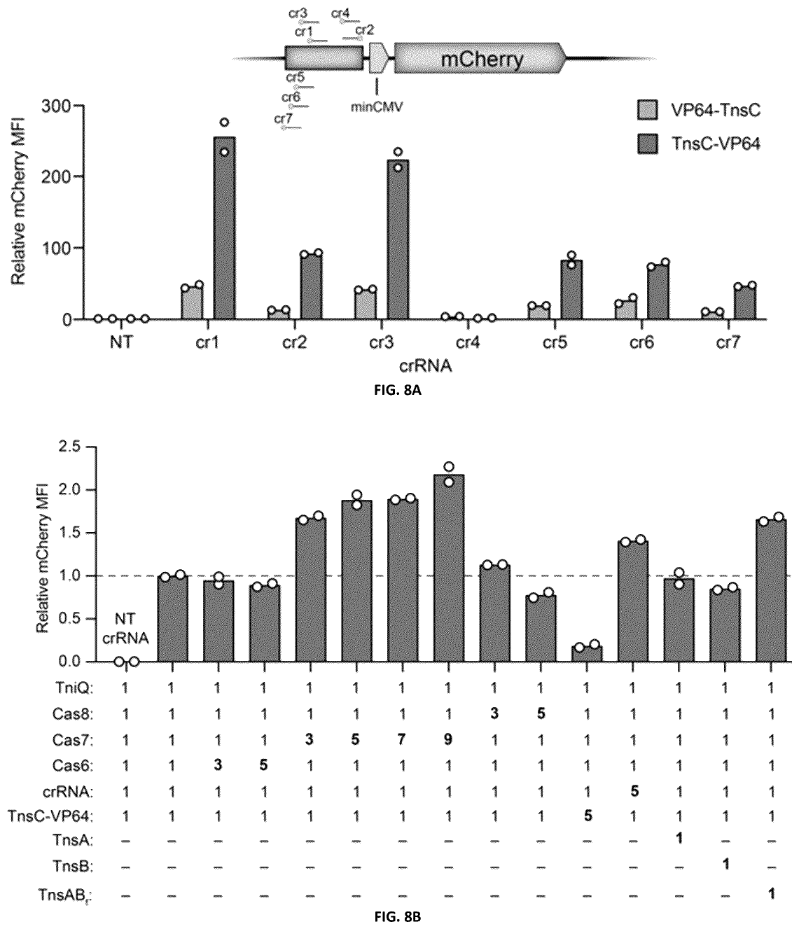

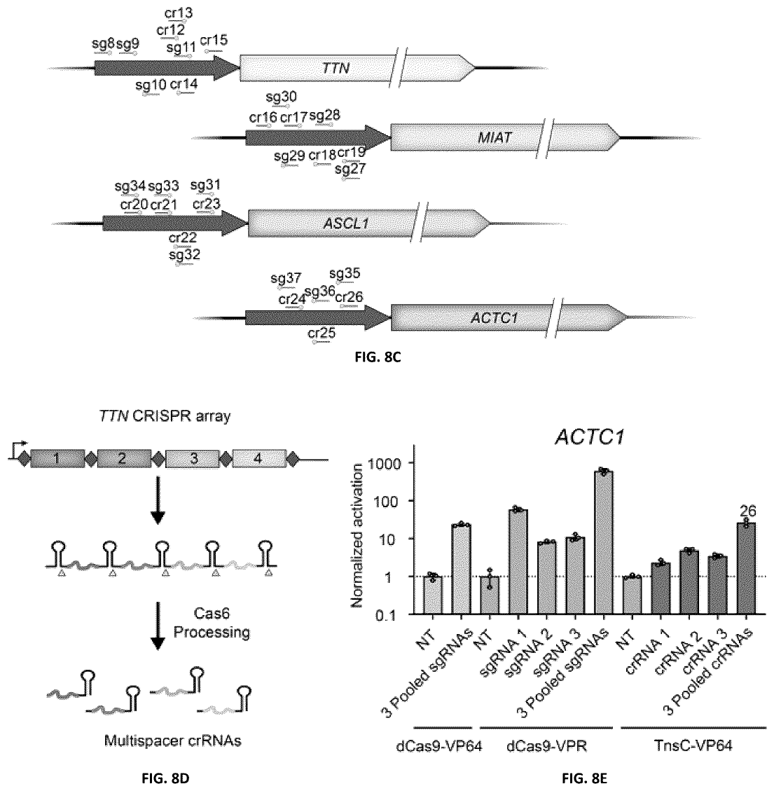

Multivalent assembly of TnsC may be used to increase the potency of transcriptional activation in mammalian cells, while also demonstrating recruitment of a critical transposase component in a QCascade-dependent fashion (FIG. 2E). VP64 was fused to either the N- or C-terminus of TnsC, seven candidate sites upstream of the mCherry reporter gene were targeted (FIG. 8A), and the potential for TnsC to stimulate transcriptional activation was investigated. Strikingly, TnsC-VP64 activators drove substantially higher levels of mCherry activation than QCascade alone, and activation levels could be further improved by optimizing the relative amount of each expression plasmid used during transfection (FIGS. 2F, 8B). This effect was absent when TniQ was omitted or an E. coli TnsC homolog was substituted, confirming the importance of cognate TniQ-TnsC interactions. Furthermore, a TnsC ATPase mutant that prevents oligomer formation (E135A) also abolished transcriptional activation, suggesting that the observed signal requires protein oligomerization on DNA (FIG. 2F). Non-targeting controls generated undetectable mCherry MFI above background levels, demonstrating the specificity of potential TnsC filamentation in Type I-F CASTs (FIG. 2F). When probing the specificity of QCascade DNA binding, intermediate levels of transcriptional activation were retained when mismatches were tiled within the middle of the 32-bp target site, but there was a reliance on cognate pairing in the seed (positions 1-8) and PAM-distal (positions 25-32) regions (FIG. 2G).

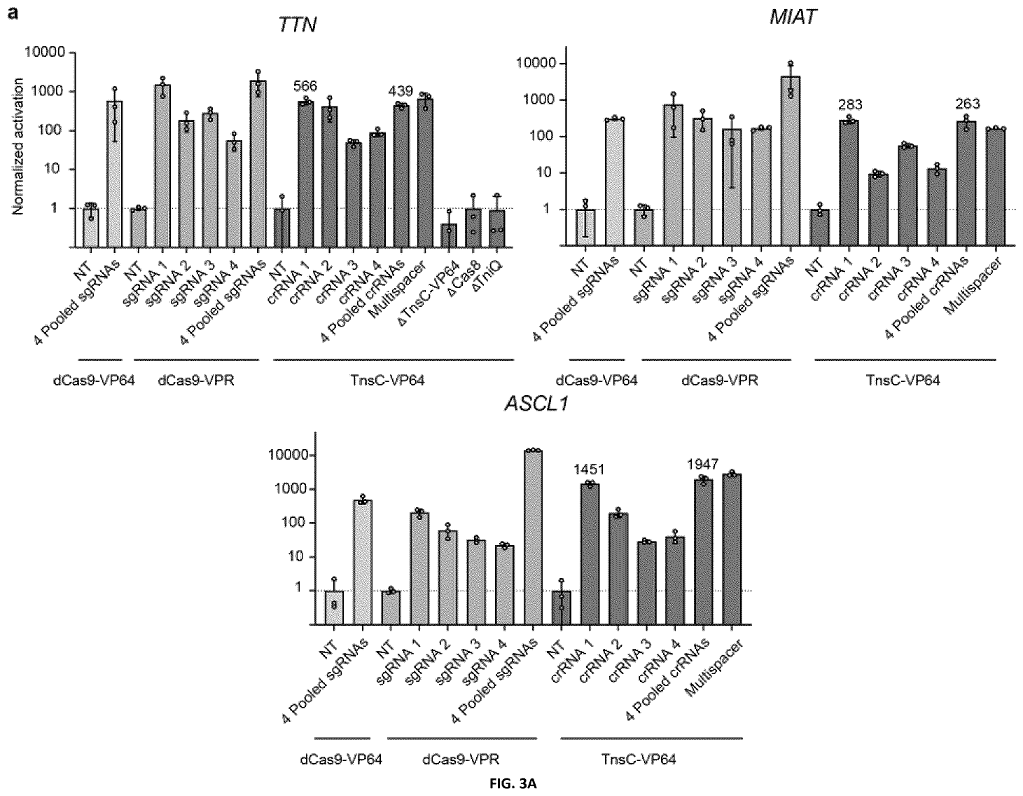

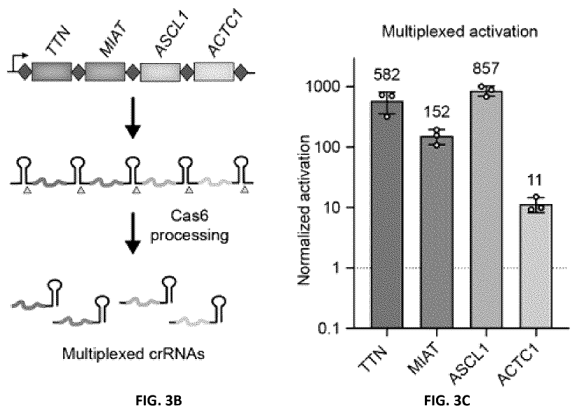

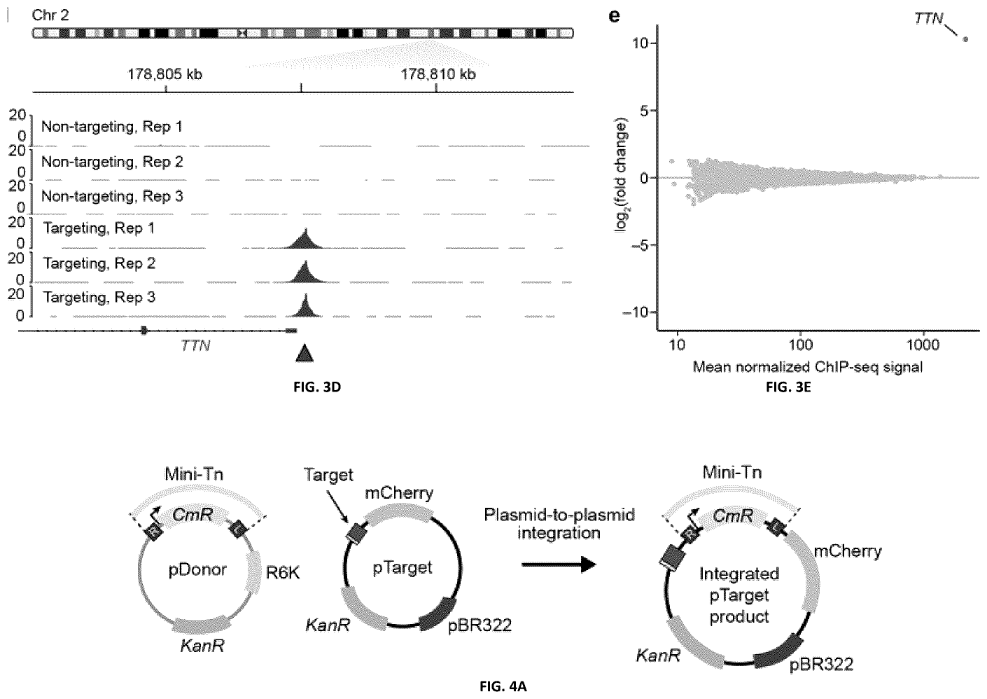

Four endogenous genes in the human genome (TTN, MIAT, ASCL1, and ACTC1), which have been previously targeted with CRISPRa using dCas9-VPR, were targeted. Three or four distinct crRNAs tiled upstream of the transcription start site were designed and delivered by either transfecting a single crRNA expression plasmid, co-transfecting multiple crRNA expression plasmids, or transfecting a single crRNA expression plasmid containing a four-spacer CRISPR array (FIG. 3A, 8C, 8D). TTN induction by TnsC-VP64 was comparable to dCas9-VP64 and dCas9-VPR activation, and the presence of Cas8 and TniQ facilitated induction (FIG. 3A). Potent activation was seen on other genomic targets ranging from 200-fold (MIAT) to >1000-fold (ASCL1), highlighting the programmability of the multimeric system (FIG. 3A), though other sites showed more moderate activation (FIG. 8E). Furthermore, the ability to utilize a multiplexed CRISPR array containing four spacers that each targeted a different gene to achieve robust transcriptional activation of all 4 genes (TTN, MIAT, ASCL1, and ACTC1) in the same cell population was demonstrated at levels comparable to activation achieved by single spacer CRISPR arrays (FIGS. 3B and 3C).

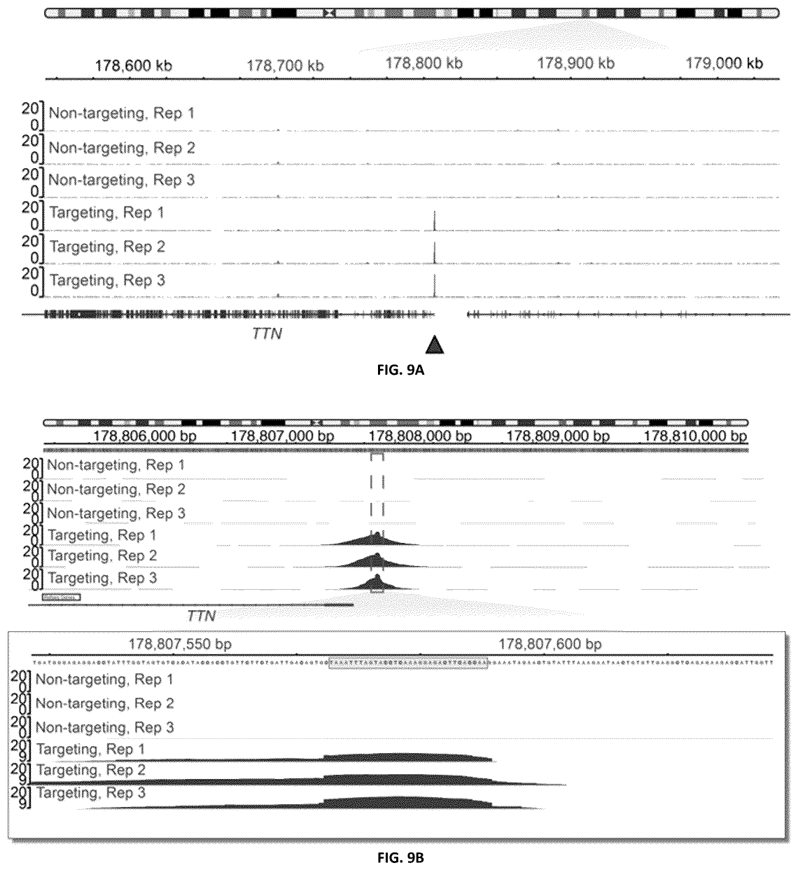

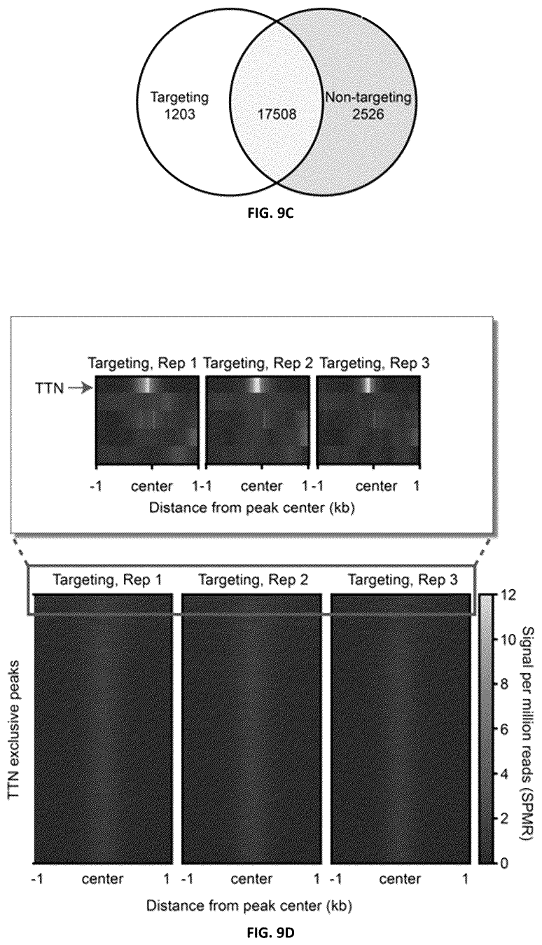

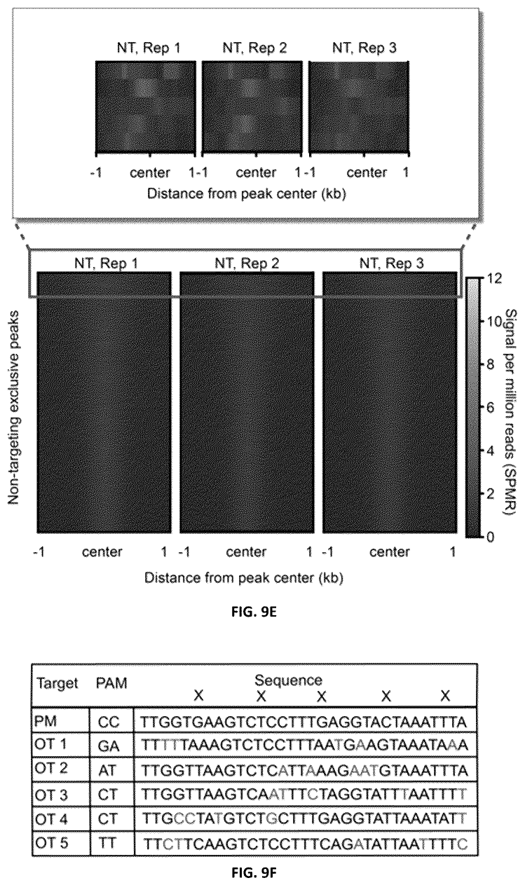

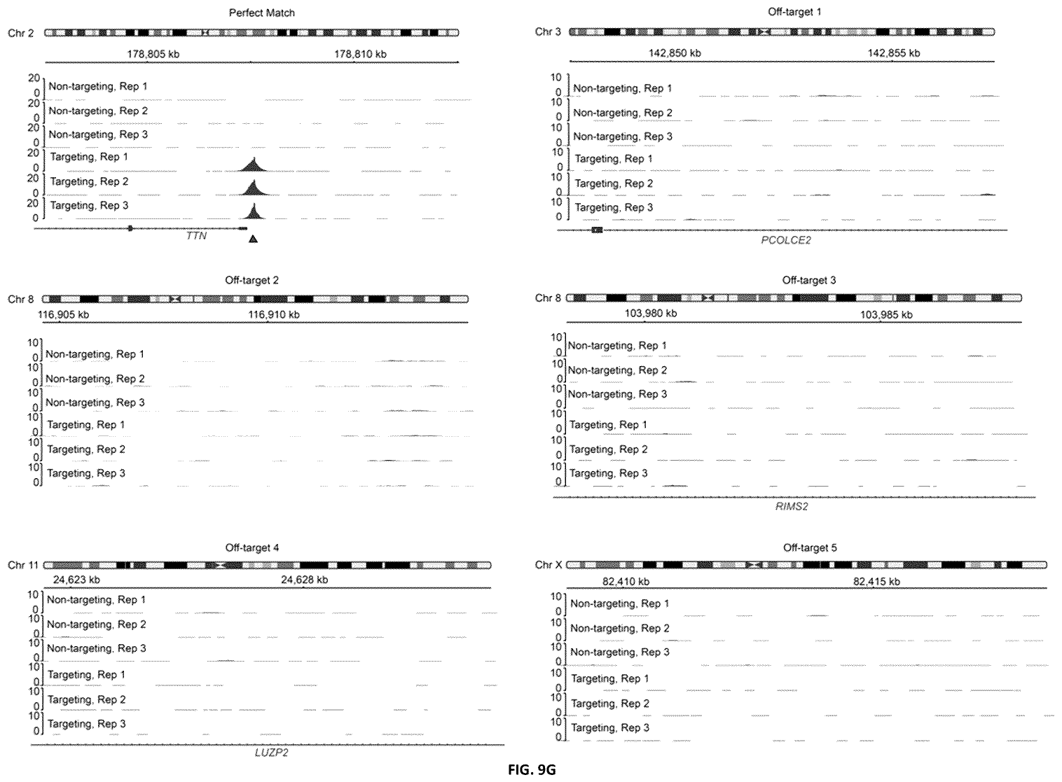

The fidelity of TnsC recruitment was investigated by performing ChIP-seq after co-transfecting plasmids encoding FLAG-tagged TnsC, protein components of QCascade, and a TTN-specific crRNA. Analysis of the resulting data revealed a sharp peak directly upstream of the TTN transcriptional start site (TSS) at the expected target site, which was absent in non-targeting (NT) samples transfected with a crRNA containing a spacer not found in the human genome (FIGS. 3D, 9A, 9B). To assess off-target binding, all peaks in both targeting and non-targeting conditions were analyzed across three biological replicates and differential binding analysis was performed, revealing only a single region at the TTN promoter that exhibited significantly different binding affinity between both conditions (FDR<0.05), highlighting the specificity of Type I-F CAST assembly (FIGS. 3E and 9C). Heatmap analysis of additional peaks that were called in either targeting or non-targeting conditions revealed low enrichment values, and a further manual inspection of 5 potential off-target sites that exhibited high similarity to the TTN spacer sequence lacked any detectable signal enrichment in the ChIP-seq datasets (FIG. 9D-9G). These results indicate that TnsC binds target sites marked by QCascade with high-fidelity, and that the intrinsic ability of TnsC to form ATP-dependent oligomers enables multiple copies of an effector protein to be delivered to genomic sites targeted by a single guide RNA.

This programmable, multivalent recruitment represents an exciting opportunity to further develop genome and transcriptome engineering tools that benefit from RNA-guided DNA binding of an effector ATPase. In the context of efforts to reconstitute CAST systems, TnsC-mediated transcriptional activation provided compelling evidence that both CRISPR- and transposon-associated protein components can be functionally assembled at plasmid and genomic target sites in a highly specific and programmable manner.

### Example 5

### RNA-Guided Episomal DNA Integration in Human Cells

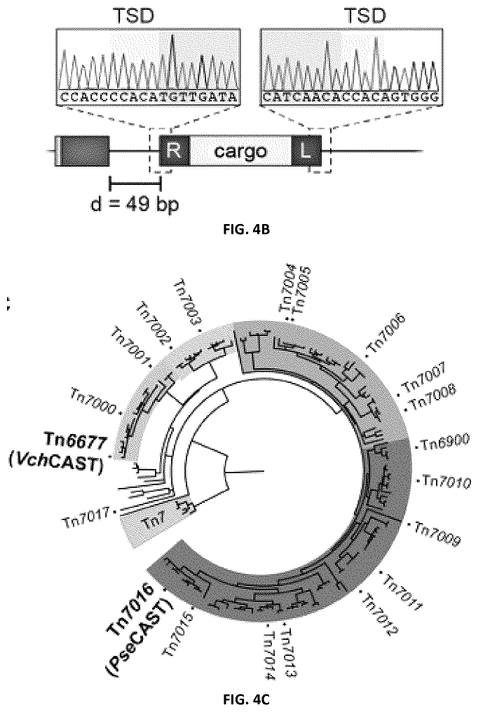

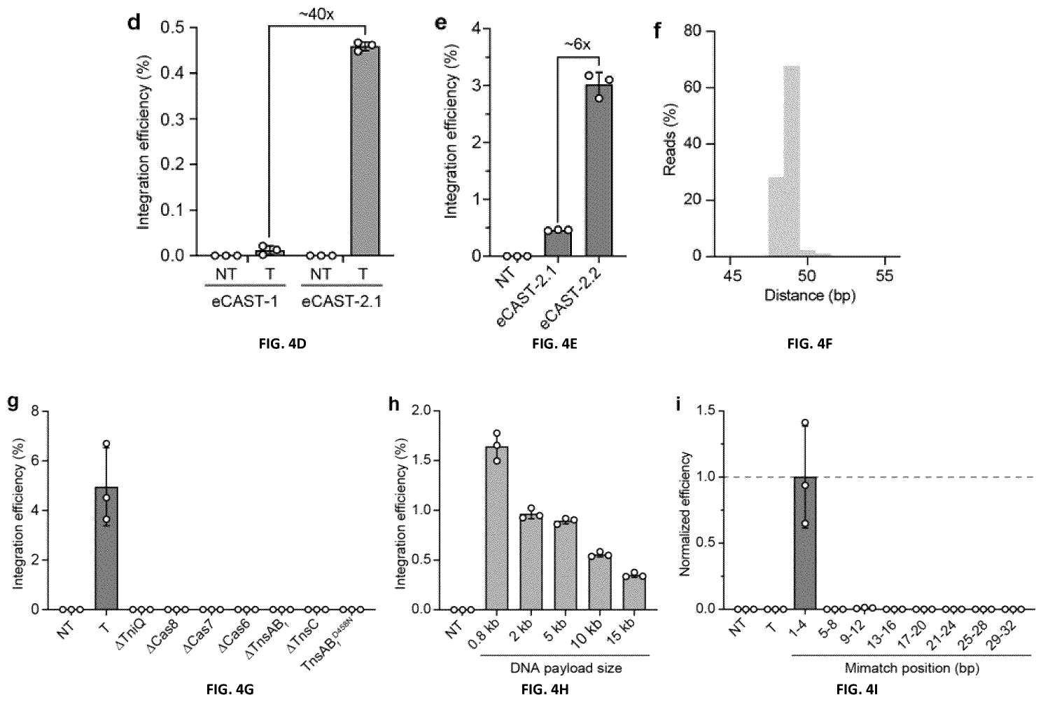

A promoter-driven chloramphenicol resistance cassette (CmR) was cloned within the mini-transposon of a donor plasmid (pDonor) and then the same sequence on the mCherry reporter plasmid (pTarget) that was used in transcriptional activation experiments was targeted. Upon successful transposition in HEK293T cells, integrated pTarget products will carry both CmR and KanR drug markers and can thus be selected for by transforming E. coli with plasmid DNA isolated from transfected cells (FIG. 4A). Importantly, a pDonor backbone that cannot be replicated in standard E. coli strains was used, reducing background from unreacted plasmids. A TnsAB fusion protein (TnsAB_{f}) that contains an internal bipartite NLS and maintains wild-type activity in E. coli was used (FIG. 6C), thereby reducing the number of unique protein components; this modified system is hereafter referred to as engineered CAST-1 (eCAST-1).

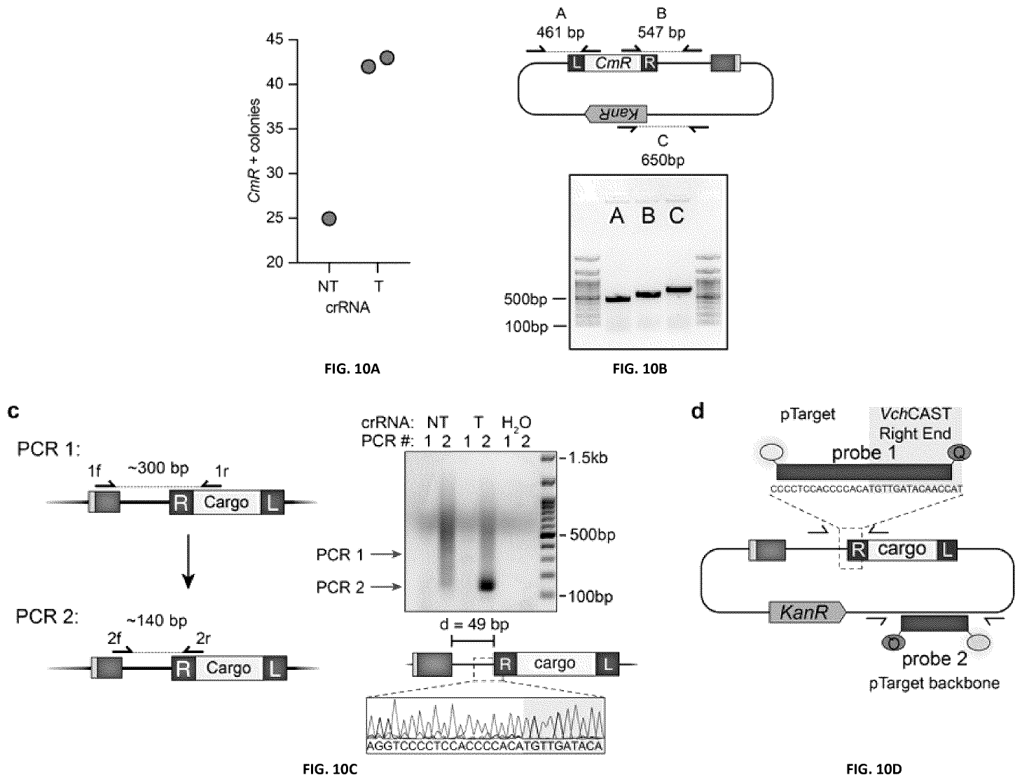

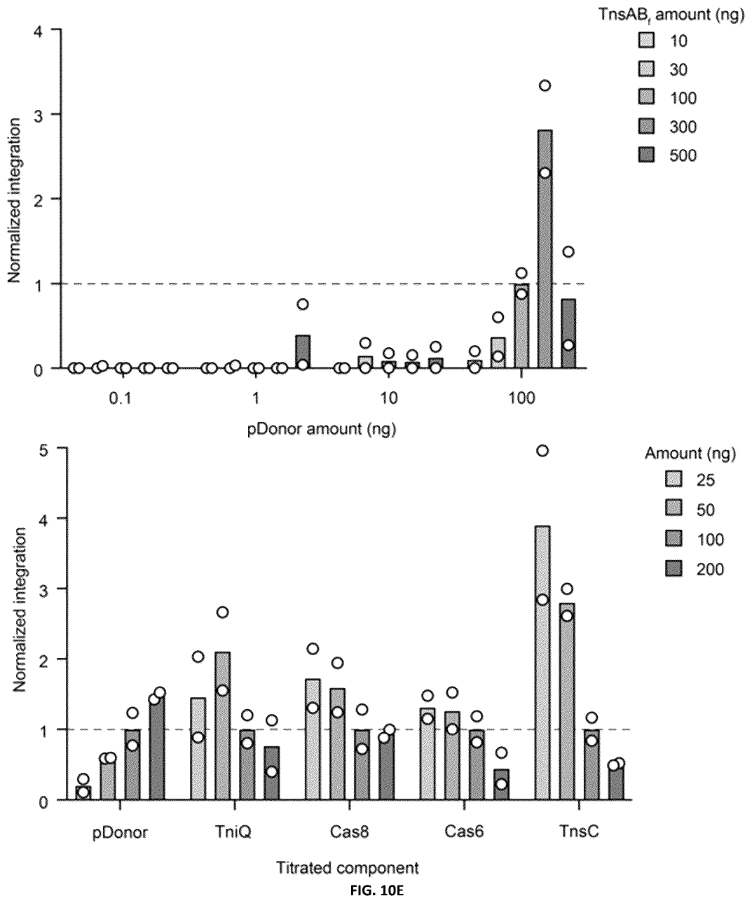

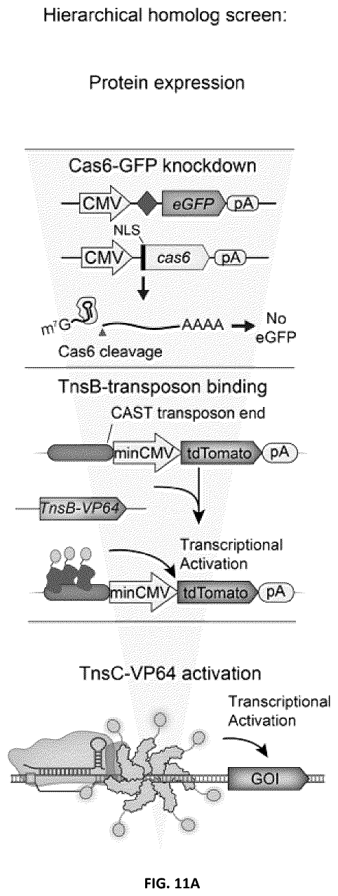

After transfecting HEK293T cells with pDonor, pTarget, and all protein-RNA expression plasmids, purifying the plasmid mixture from cells, and using the mixture to transform E. coli, the emergence of colonies that were chloramphenicol resistant was observed, which outnumbered the corresponding colonies obtained from experiments using a non-targeting crRNA that did not match pTarget (FIG. 10A). Junction PCR was performed on select colonies and bands of the expected size were obtained, which subsequent Sanger sequencing confirmed were integration products arising from DNA transposition 49-bp downstream of the target site (FIG. 4B), as expected. Further analyses of individual clones revealed the expected junction sequences across both the transposon left and right ends (FIG. 10B). The same products could be detected by nested PCR directly from HEK293T cell lysates (FIG. 10C), and a sensitive TaqMan probe-based qPCR strategy was used to quantify integration events from lysates by detecting site-specific, plasmid-transposon junctions (FIG. 10D). Using this approach, an initial optimization screen was performed by varying the relative amounts of expression and pDonor plasmids and efficiencies were greatest with low levels of pTnsC and high levels of pTnsAB_{f} and pDonor (FIG. 10E). Absolute efficiencies of plasmid-to-plasmid integration with this eCAST-1 system from V. cholerae remained <0.1%.

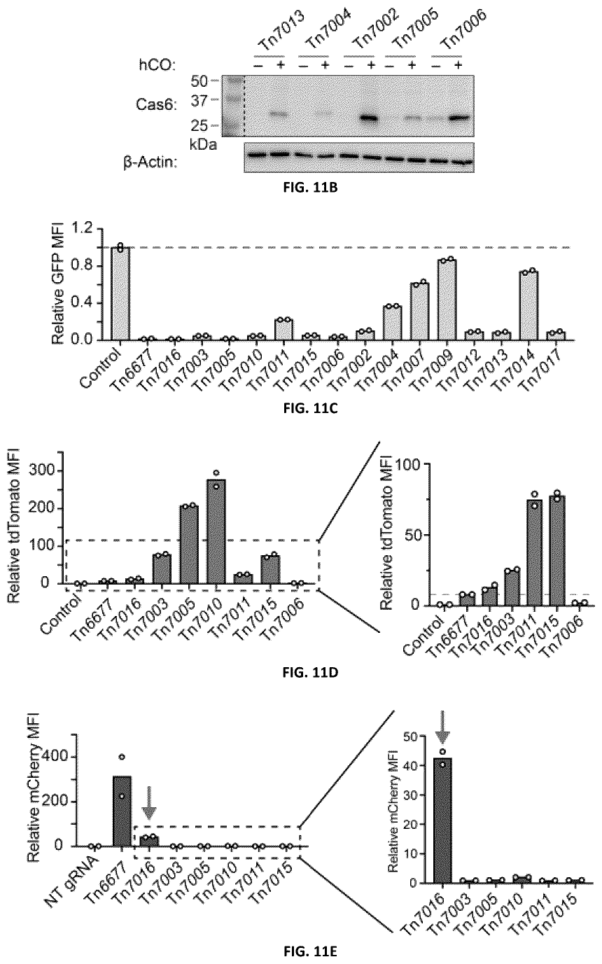

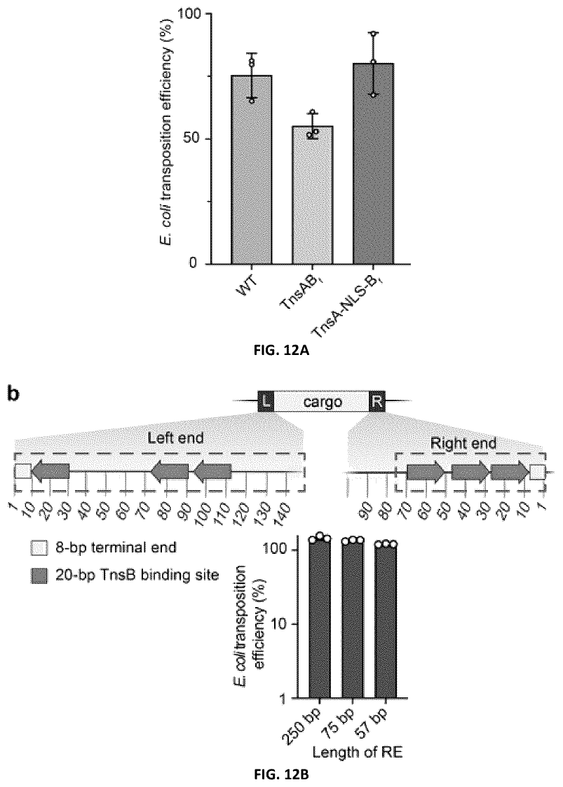

The bioinformatic mining and experimental characterization of 18 new Type I-F CRISPR-associated transposons (denoted Tn7000-Tn7017), many of which exhibited high-efficiency and high-fidelity RNA-guided DNA integration in E. coli (FIG. 4c), was used in a hierarchical screening approach to uncover variants with improved activity in human cells (FIG. 11A). Briefly, the screening approach involved filtering based on robust activity in three key areas: (i) crRNA biogenesis by Cas6, assessed using the GFP knockdown assay; (ii) transposon DNA binding by TnsB, assessed using a tdTomato reporter assay; and (iii) transcriptional activation by TnsC-VP64, assessed using the mCherry reporter assay. In all cases, genes were human codon optimized, which often facilitated achieving strong expression (FIG. 11B), and tagged with NLS sequences on the same termini as for Tn6677 (VchCAST). The majority of systems exhibited efficient crRNA biogenesis and transposon DNA binding activity that was similar to that observed with Tn6677 (FIGS. 11C-11D). Interestingly, of those systems selected for testing in transcriptional activation experiments, only Tn7016 showed reproducible induction of mCherry expression, albeit at levels ˜8-fold lower than Tn6677 (FIG. 11E).

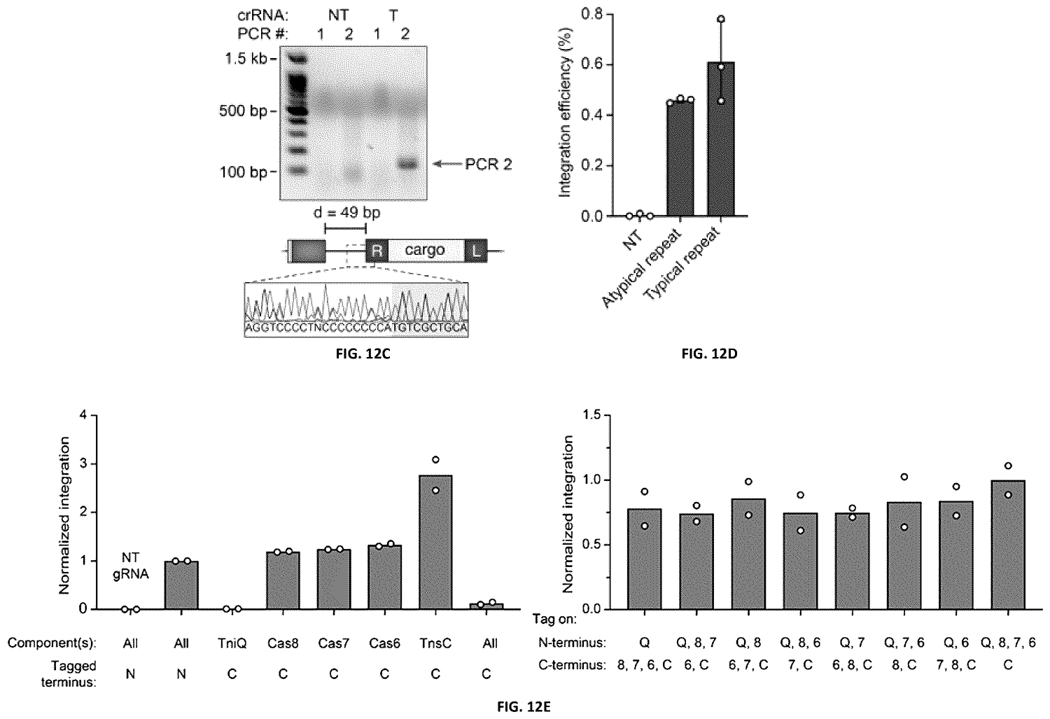

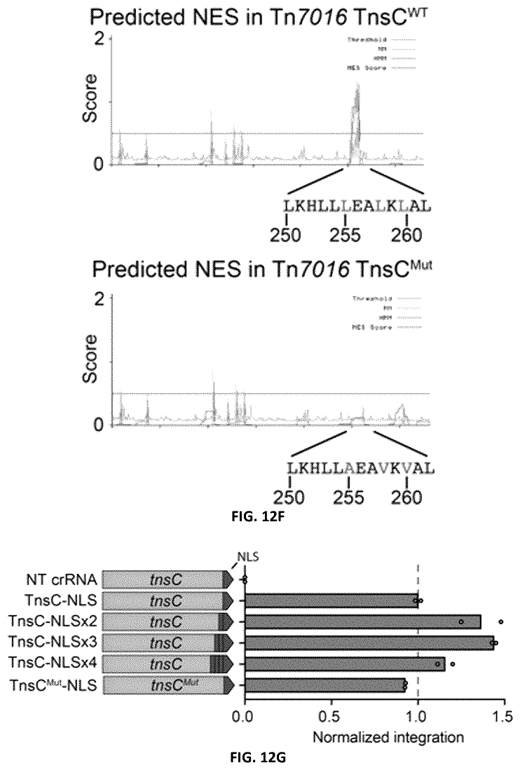

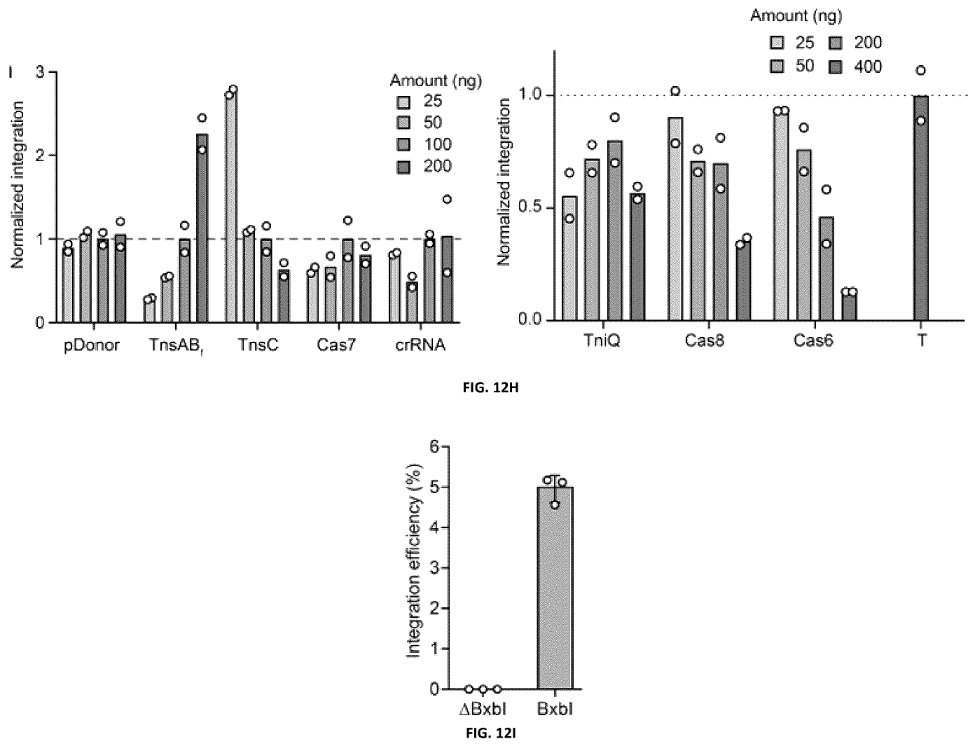

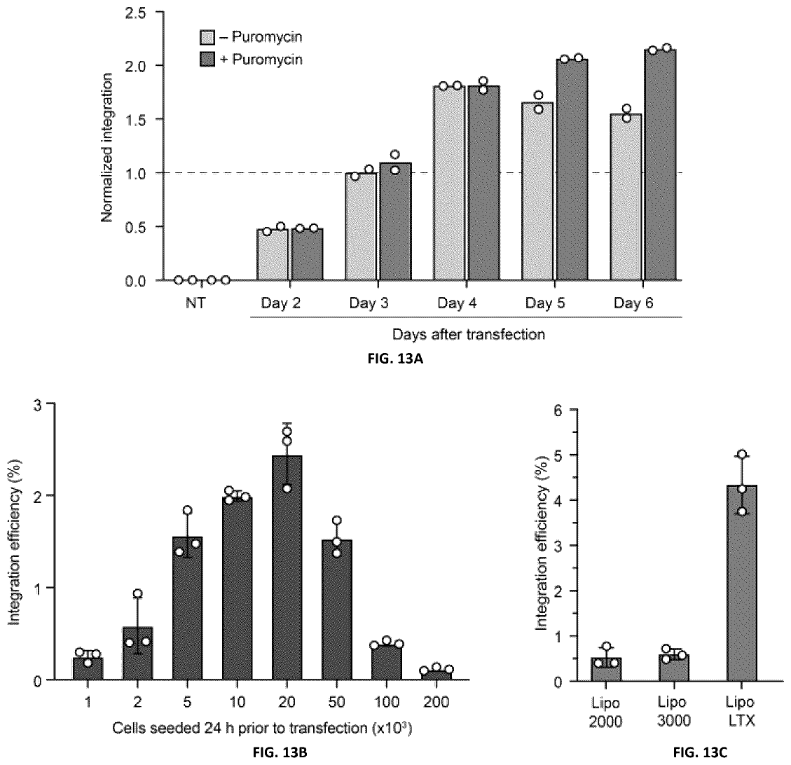

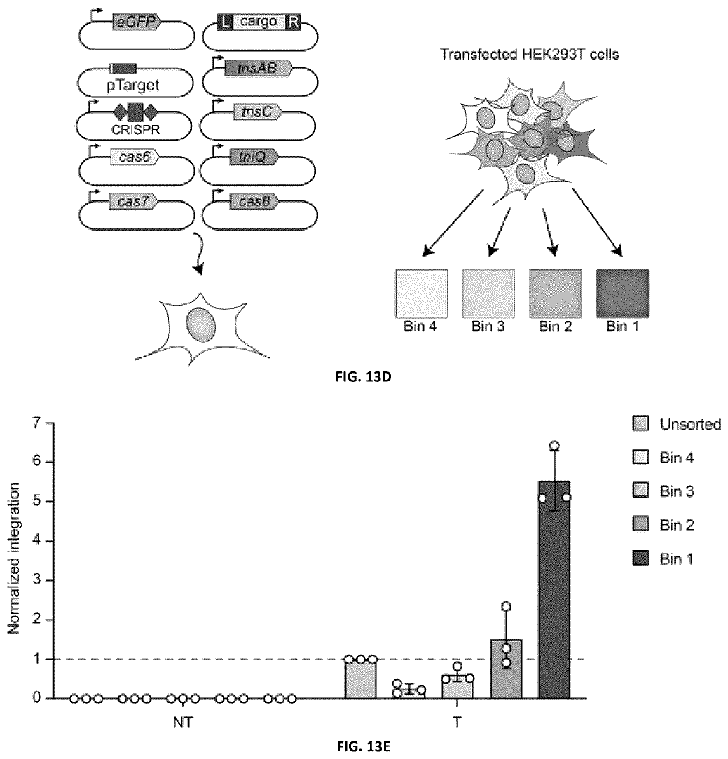

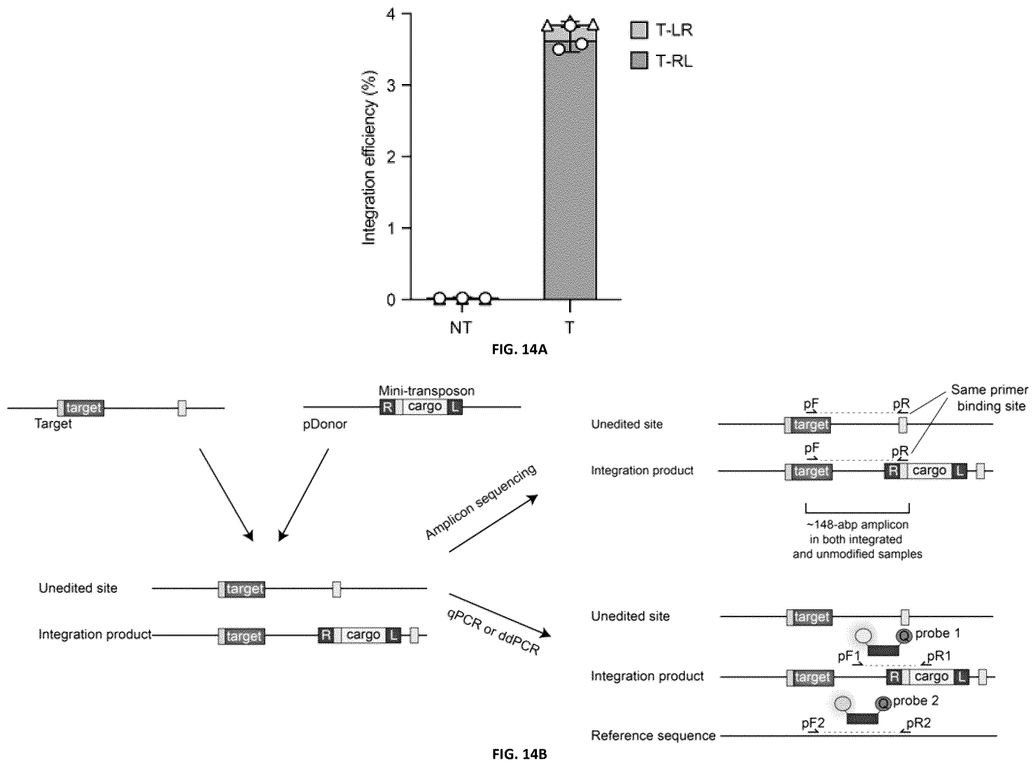

After verifying that fusing TnsA and TnsB from Tn7016, a 31-kb transposon from Pseudoalteromonas sp. S983 (PseCAST), and with an internal NLS retained function, and optimizing the length of left and right transposon ends (FIGS. 12A-12B), plasmid-to-plasmid transposition assays were repeated in HEK293T cells. Strikingly, the engineered Pseudoalteromonas CAST (eCAST-2.1) was ˜40-fold more active than eCAST-1 when tested under unoptimized conditions (FIGS. 4D and 12C). To further improve integration efficiencies, the design of the crRNA, location of NLS tags, and relative amounts of each expression plasmid were systematically varied; the resulting eCAST-2.2 yielded a further ˜6-fold improvement to reach levels of 3-5% integration, and PCR followed by Sanger or Illumina sequencing analysis confirmed the expected site of integration 49-bp downstream of the target (FIGS. 4E, 4F, and 12D-12H). Of note, these efficiencies were comparable to integration efficiencies achieved with BxbI under similar plasmid-to-plasmid conditions (FIG. 12I). Peak integration occurred 4-6 days post-transfection, with the efficiency exhibiting sensitivity to both cell density and the choice of cationic lipid delivery method (FIGS. 13A-13C). The observed integration efficiency was increased by >5-fold upon co-transfection of a GFP transfection marker and separately analyzing sorted cells exhibiting high GFP fluorescence levels, suggesting that activity was dependent not only the stoichiometry of the transfected plasmids but also the plasmid dosage across the population of cells (FIGS. 13D-13E).

Integration was dependent on a targeting crRNA and the presence of all protein components, including an intact TnsB active site (FIG. 4G), and functioned with genetic payloads spanning 1-15 kb in size, albeit with a ˜3-fold decrease in efficiency with larger payloads (FIG. 4H). A panel of mismatched crRNAs was generated in which mutations were tiled along the length of the 32-nt guide, and activity was ablated regardless of the location (FIG. 4I), indicating a greater degree of discrimination than that observed in activation experiments utilizing VchCAST in activation experiments or in E. coli. An alternative qPCR approach was used to confirm that integration orientation for eCAST-2.2 was highly biased towards T-RL, as expected from prior bacterial integration data (FIG. 14A). An NGS-based amplicon sequencing approach was used to quantify all integration events at the expected insertion site (FIGS. 14B-14C) and droplet digital PCR (ddPCR) corroborated the quantitative data obtained from TaqMan qPCR (FIG. 14D).

### Example 6

### RNA-Guided Episomal DNA Integration in Human Cells

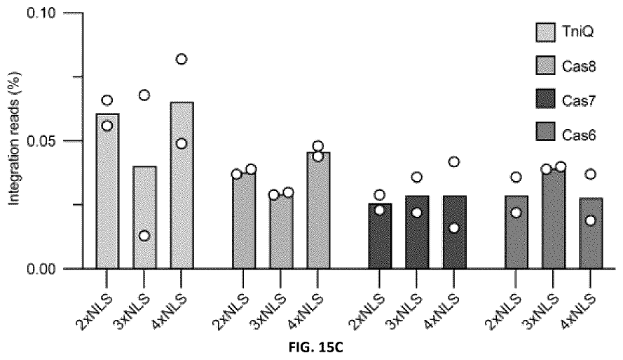

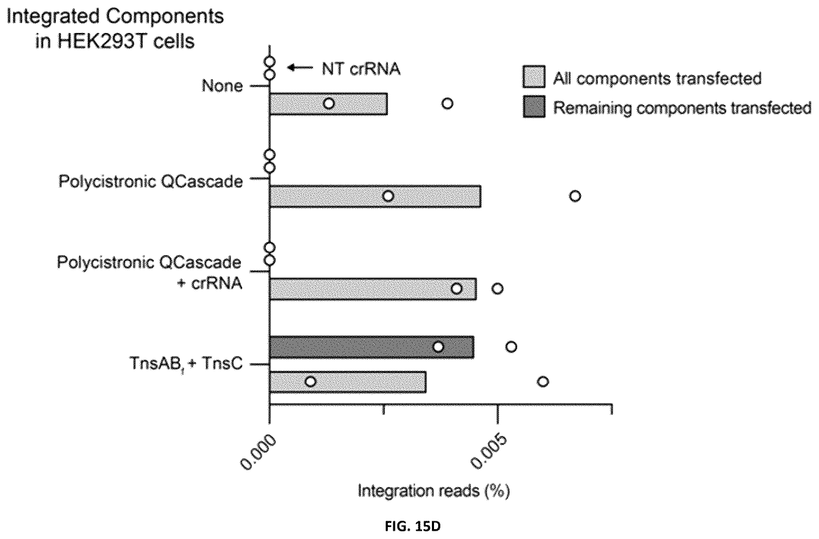

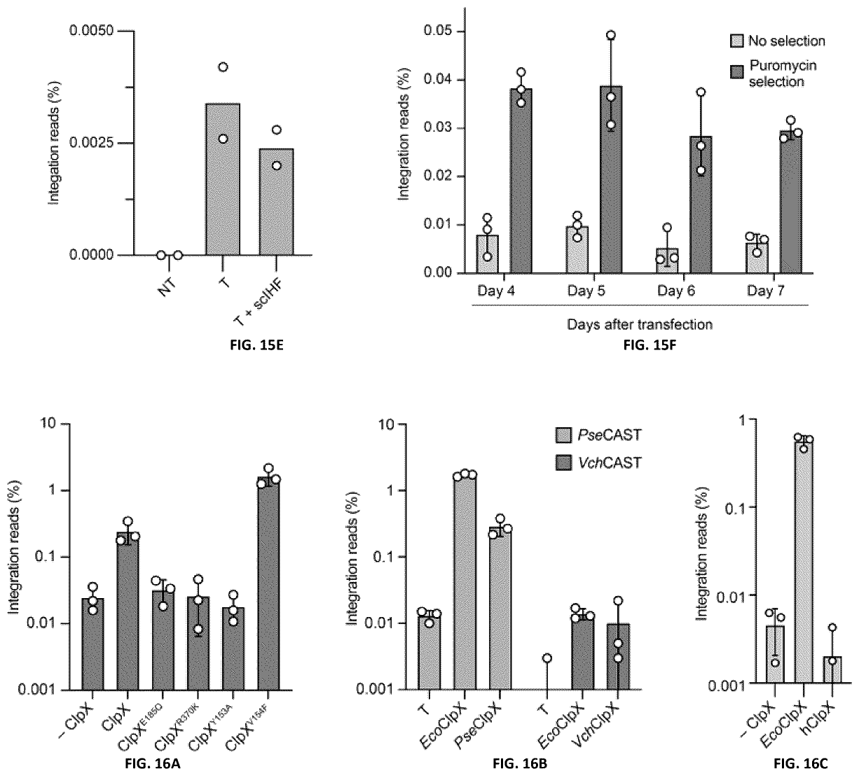

A panel of guide sequences targeting the AAVS1 safe-harbor locus were screened via a plasmid-to-plasmid integration assay, in which 32-bp target sites derived from AAVS1 were cloned into pTarget and existing assays were leveraged to identify two active crRNAs that outperformed the original plasmid-specific crRNA (FIG. 15A). When the AAVS1 locus was tested for genomic integration using a nested PCR strategy, RNA-guided DNA integration products were identified that again maintained the expected 49-bp distance dependence from the target site (FIG. 5A). However, detection was often not consistent across biological replicates, suggesting that integration efficiencies were near the limit of detection. An NGS-based amplicon sequencing method established in the prior plasmid-based assays yielded reproducible efficiencies on the order of ˜0.005% (FIGS. 5B and 14B).

An additional 8 sites were targeted across the genome, with 1-3 crRNAs per locus, and detected integration at efficiencies that varied but were generally ˜0.01% (FIG. 5C). Attempts to increase the efficiency further through simplified delivery of a polycistronic QCascade expression vector, serial additions of extra NLS sequences, constitutive expression of the targeting machinery, inclusion of bacterial IHFa/b, or phenotypic drug selection to enrich for integration events (FIGS. 15B-15F) did not reduce the large, 100-1,000× discrepancy between observed integration efficiencies at plasmid and genomic target sites. Although differences in chromatinization remained a distinct possibility. Without being bound by theory, the discrepancy might be due to potential toxicity of genomic integration intermediate products.

To test if CAST systems might utilize bacterial ClpX, or some other accessory factor, for active mechanical disassembly of the PTC, human cells were co-transfected with eCAST-2.2 components and a plasmid expressing NLS-tagged E. coli ClpX (EcoClpX), collectively referred to as eCAST-3. Remarkably, genomic integration efficiencies increased by ˜100× in a ClpX dose-responsive manner, albeit with observable ClpX-induced cellular toxicity, whereas plasmid integration efficiencies were unaffected (FIGS. 5E and 5F). To investigate if the effect was specific to ClpX, other bacterial unfoldases were tested, including ClpA and ClpB, and found that ClpX was the only tested ATPase that enhanced genomic integration. ClpP, which functions as the peptidase component within the ClpXP protease complex, had no effect on integration, either alone or in combination with ClpX, suggesting that protein unfolding, but not protein degradation, is sufficient (FIG. 5G). When point mutations that ablate ATP hydrolysis (E185Q or R370K) or substrate engagement (Y153A) were introduced, ClpX failed to enhance genomic integration (FIG. 16A), further supporting the mechanistic link between ATPase-driven protein unfolding and PTC disassembly. ClpX is highly conserved across bacterial species, and the homolog from Pseudoalteromonas (80% amino acid identity) also stimulated integration, albeit to a slightly lesser extent that EcoClpX (FIG. 16B); NLS-tagged human ClpX, which normally functions in the mitochondria, had no effect on integration (FIG. 16C). Interestingly, genomic integration with eCAST-1 (VchCAST) was reproducibly detectable in the presence of EcoClpX or VchClpX but not in its absence, indicating a consistent effect across Type I-F CAST systems, though lower intrinsic activity of VchCAST was observed similar to plasmid-to-plasmid integration assays (FIG. 16B). Collectively, these results suggest that PTC disassembly may be a bottleneck limiting integration into genomic target sites, and identify ClpX as an accessory factor that acts to unfold one or more components within the CAST transpososome (FIG. 16D

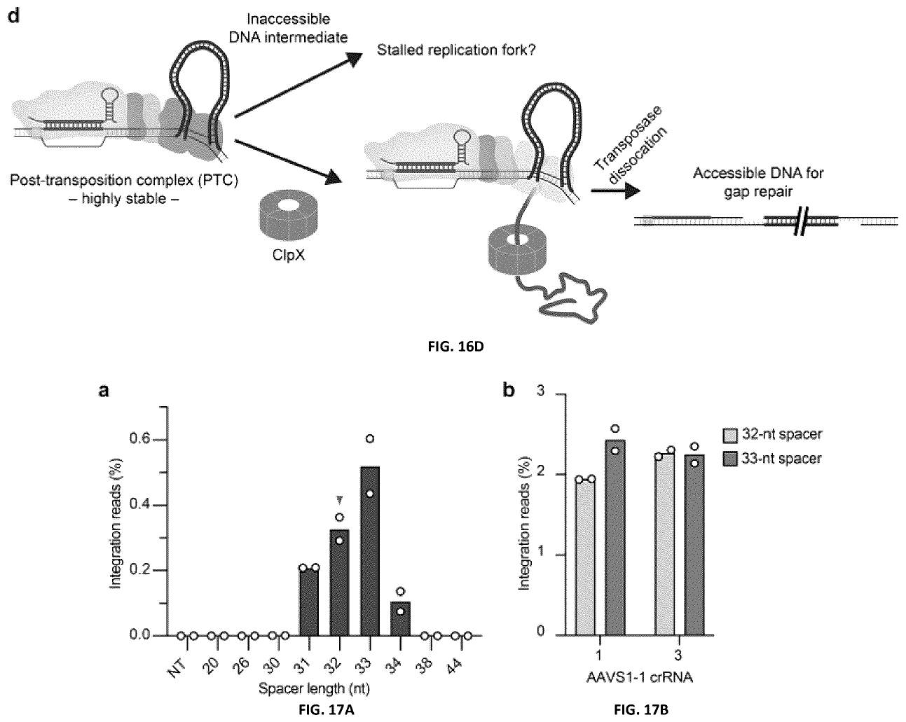

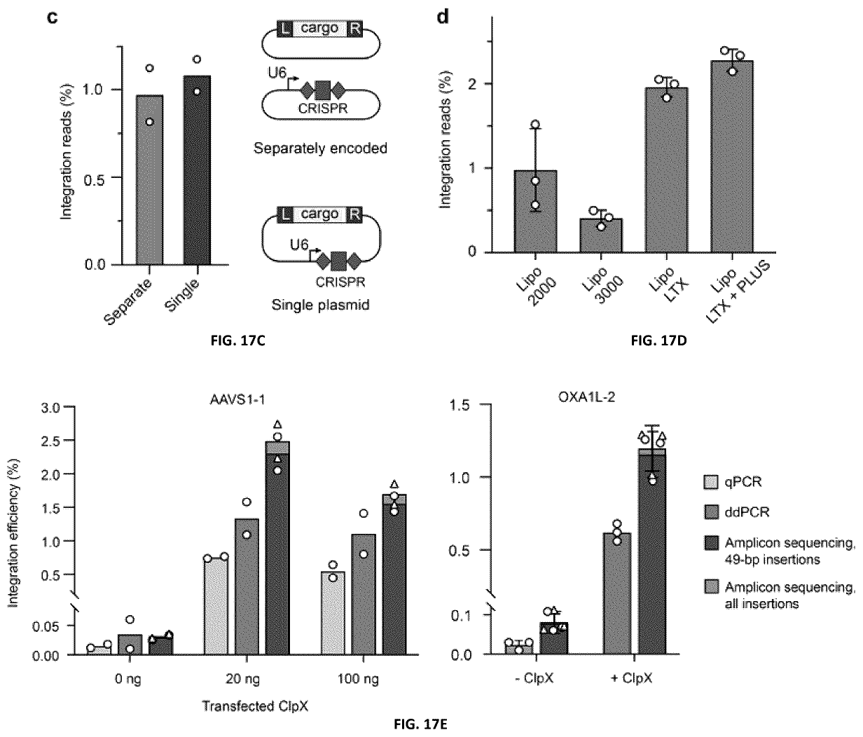

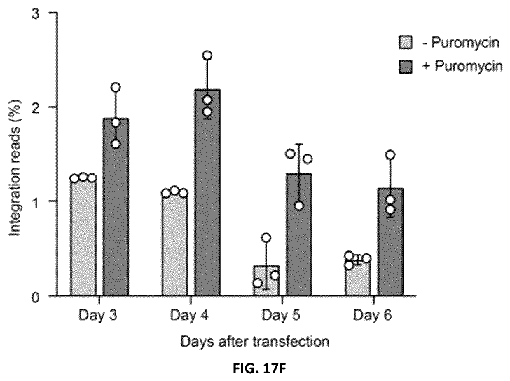

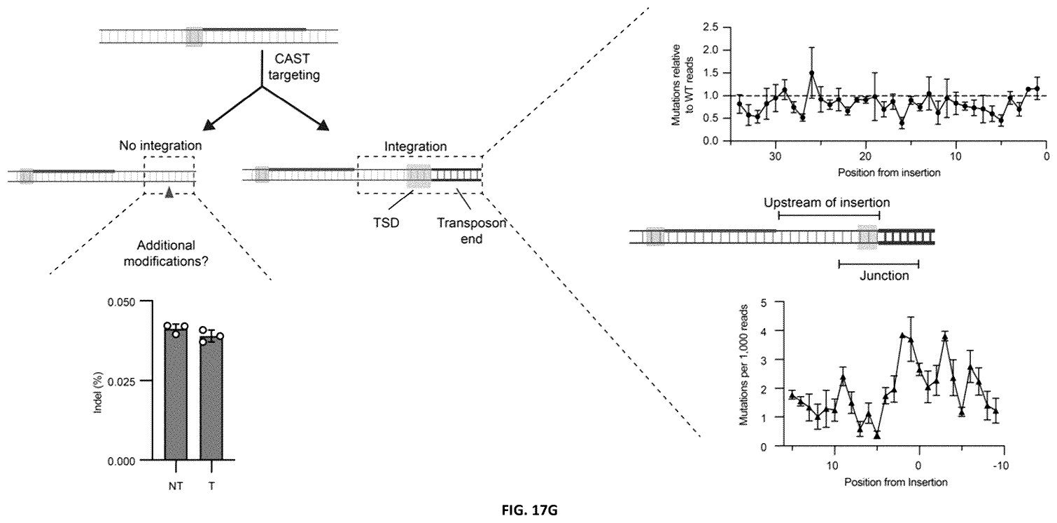

Single-digit genomic integration efficiencies at the AAVS1 locus allowed exploration of other parameters of eCAST-3 design and delivery. crRNAs functioned best with 33-nt spacers on both plasmid and genomic targets (FIGS. 17A-17B), and that transfections could be simplified by placing the U6-driven crRNA cassette directly on pDonor without an adverse effect on activity (FIG. 17C). Integration could be further improved with the appropriate selection of cationic lipid formulation (FIG. 17D), or by selecting/sorting cells that were co-transfected with either a drug or fluorescent marker, with efficiencies reaching ˜5% as measured by amplicon-sequencing and ddPCR (FIGS. 5H and 17E-17F). Inspection of the next-generation sequencing data revealed an absence of indels above background (˜0.04% sequencing error) at unedited target sites, and an absence of detectable mutations surrounding genome-transposon junctions (FIG. 17G), suggesting that CAST systems are less prone to the range of byproducts common to Cas9 nuclease and nickase-based approaches.

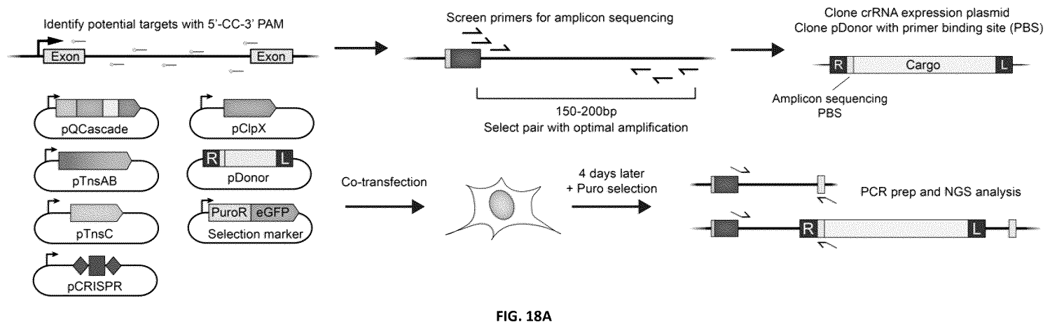

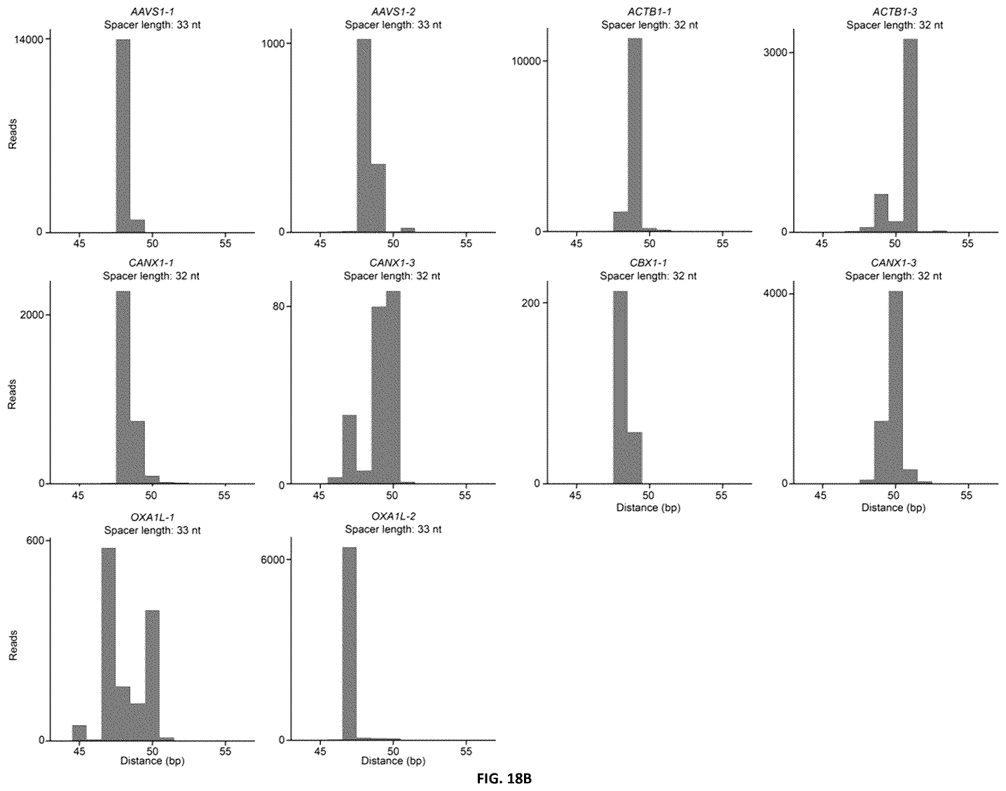

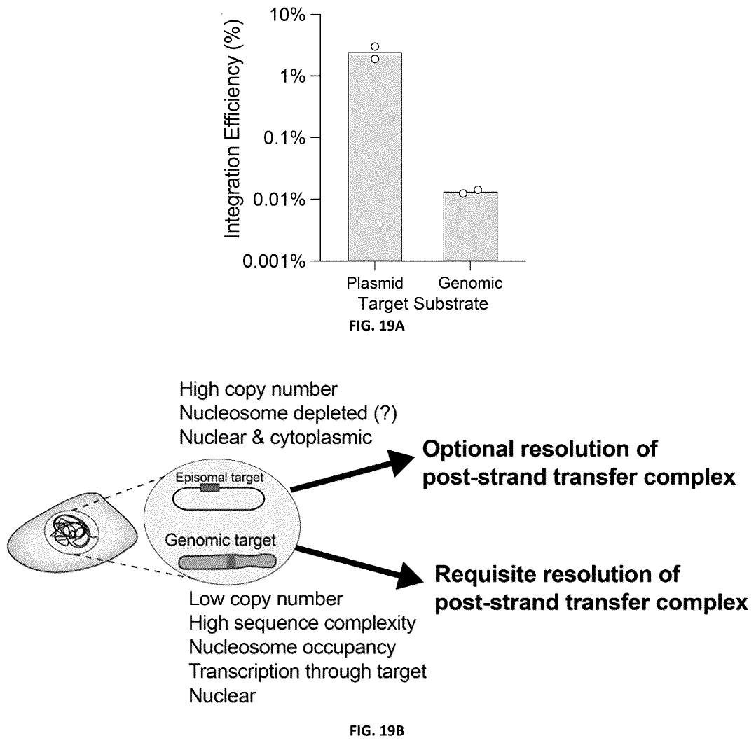

Lastly, previously targeted sites across the human genome were revisited and assessed for integration efficiency to test the generalizability of ClpX enhancement (FIG. 18A). Strikingly, a 10-600-fold increase in integration efficiencies was observed across all tested loci (FIG. 5I), with a consistent preference for insertions ˜49-bp downstream of the crRNA-matching target site (FIG. 18B), as first reported in E. coli studies.

TABLE 1
Sequence and description of plasmids

| | | SEQ |
|---|---|---|
| | | ID |
| Plasmid ID | Plasmid description | NO |
| pSL0341 | pTarget | 12 |
| pSL0454 | Pse Cascade VP64-Cas7 | 13 |
| pSL0532 | Pse I-E Targeting crRNA | 14 |
| pSL0534 | Pse I-E NT crRNA | 15 |
| pSL2276 | Pse I-E_DR-eGFP | 16 |
| pSL2277 | Tn6677_DR-eGFP | 17 |
| pSL2279 | Pse I-E pCas6 | 18 |
| pSL812 | Vch stuffer crRNA | 19 |
| pSL2620 | Vch pTniQ | 20 |
| pSL2621 | Vch pCas8 | 21 |
| pSL2622 | Vch pCas7 | 22 |
| pSL2623 | Vch pCas6 | 23 |
| pSL2645 | Vch pTnsC | 24 |
| pSL2669 | Vch pTnsABf | 25 |
| pSL2693 | Vch pVP64-Cas7 | 26 |
| pSL2783 | Vch pTnsC-VP64 | 27 |
| pSL3617 | Pse stuffer crRNA | 28 |
| pSL2912 | Pse pTniQ | 29 |
| pSL2913 | Pse pCas8 | 30 |
| pSL2914 | Pse pCas7 | 31 |
| pSL2915 | Pse pCas6 | 32 |
| pSL3718 | PseQCascade | 33 |
| pSL3713 | Pse pTnsC-3xNLS | 34 |
| pSL3402 | Pse pTnsA-NLS-Bf | 35 |
| pSL3626 | Vch pDonor | 36 |
| pSL3637 | Pse pDonor | 37 |
| pSL3927 | Pse pDonor | 38 |
| pSL3744 | Pse pDonor | 39 |
| pSL3936 | Pse pDonor | 40 |
| pSL4103 | Pse pDonor | 41 |
| pSL4575 | Pse pDonor | 42 |
| pSL3815 | Pse pDonor | 43 |
| pSL4070 | Pse pDonor | 44 |
| pSL4071 | Pse pDonor | 45 |
| pSL4072 | Pse pDonor | 46 |
| pSL4762 | Pse pDonor | 47 |
| pSL4763 | Pse pDonor | 48 |
| pSL4764 | Pse pDonor | 49 |
| pSL4765 | Pse pDonor | 50 |
| pSL4766 | Pse pDonor | 51 |
| pSL4767 | Pse pDonor | 52 |
| pSL4768 | Pse pDonor | 53 |
| pSL4774 | Pse pDonor | 54 |
| pSL4775 | Pse pDonor | 55 |
| pSL4776 | Pse pDonor | 56 |
| pSL4777 | Pse pDonor | 57 |
| pSL4165 | EcoClpX | 58 |
| pSL4166 | pcDNA3.1_ClpX_BP-NLS | 59 |
| pSL4393 | EcoClpX | 60 |
| pSL4394 | PseClpX | 61 |
| pSL4395 | VchClpX | 62 |
| pSL4396 | hClpX | 63 |
| pSL3410 | Target plasmid | 64 |
| pSL1236 | pDonor | 65 |
| pSL0828 | pQCascade, WT | 66 |
| pSL1014 | pQCascade, NT | 67 |
| pSL1478 | pQCascade, NLS-Cas8 | 68 |
| pSL1479 | pQCascade, Cas8-T2A | 69 |
| pSL1051 | pQCascade, NLS-Cas7 | 70 |
| pSL1480 | pQCascade, Cas7-T2A | 71 |
| pSL2282 | pQCascade, NLS-Cas6 | 72 |
| pSL1053 | pQCascade, Cas6-T2A | 73 |
| pSL1419 | pQCascade, NLS-TniQ | 74 |
| pSL1477 | pQCascade, TniQ-T2A | 75 |
| pSL0283 | pTnsABC | 76 |
| pSL1054 | pTnsABC, NLS-TnsA | 77 |
| pSL1055 | pTnsABC, TnsA-T2A, NLS-TnsB | 78 |
| pSL1482 | pTnsABC, TnsB-T2A | 79 |
| pSL1483 | pTnsABC, NLS-TnsC | 80 |
| pSL1484 | pTnsABC, TnsC-T2A | 81 |
| pSL1738 | pTnsABC, TnsABf | 82 |
| pSL2096 | pTnsABC, NLS-TnsABf | 83 |
| pSL2542 | pTnsABC, TnsABf_internal-NLS | 84 |
| pSL2097 | pTnsABC, TnsABf-NLS | 85 |
| pSL1021 | pEffector, No tags, NT | 86 |
| pSL1022 | pEffector, No tags, WT | 87 |
| pSL1567 | pEffector, all permissive tags | 88 |
| pSL1969 | pEffector, RPV-Cas8 | 89 |
| pSL1970 | pEffector, VP64-Cas7 | 90 |
| pSL1971 | pEffector, VPR-Cas7 | 91 |
| pSL5069 | pDonor(AAVS1), CRISPR_Pse | 92 |
| pSL5008 | pcDNA3.1_Tn7016_TnsA_BP-NLS_TnsB_Δ4 | 93 |
| pSL5009 | pcDNA3.1_Tn7016_TnsA_BP-NLS_TnsB_Δ8 | 94 |
| pSL5010 | pcDNA3.1_Tn7016_TnsA_BP-NLS_TnsB_Δ11 | 95 |
| pSL5011 | pcDNA3.1_Tn7016_TnsA_BP-NLS_TnsB_Δ15 | 96 |
| pSL5012 | pcDNA3.1_Tn7016_TnsA_BP-NLS_TnsB_Δ19 | 97 |
| pSL5013 | pcDNA3.1_Tn7016_TnsA_BP-NLS_TnsB_Δ23 | 98 |
| pSL5014 | pcDNA3.1_Tn7016_TnsA_BP-NLS_TnsB_Δ27 | 99 |
| pSL5015 | pcDNA3.1_BP-NLS_ClpA | 100 |
| pSL5016 | pcDNA3.1_BP-NLS_ClpB | 101 |
| pSL5017 | pcDNA3.1_BP-NLS_ClpP | 102 |

TABLE 2
Sequence and description of proteins

| Protein | | SEQ |
|---|---|---|
| Description | Protein sequence | ID NO |
| E. coli derived | MGMTDKRKDGSGKLLYCSFCGKSQHEVRKLIAGPSVYICDECVD | 1 |
| ClpX | LCNDIIREEIKEVAPHRERSALPTPHEIRNHLDDYVIGQEQAKKVL | |
| | AVAVYNHYKRLRNGDTSNGVELGKSNILLIGPTGSGKTLLAETLA | |
| | RLLDVPFTMADATTLTEAGYVGEDVENIIQKLLQKCDYDVQKAQ | |
| | RGIVYIDEIDKISRKSDNPSITRDVSGEGVQQALLKLIEGTVAAVPP | |
| | QGGRKHPQQEFLQVDTSKILFICGGAFAGLDKVISHRVETGSGIGF | |
| | GATVKAKSDKASEGELLAQVEPEDLIKFGLIPEFIGRLPVVATLNE | |
| | LSEEALIQILKEPKNALTKQYQALFNLEGVDLEFRDEALDAIAKKA | |
| | MARKTGARGLRSIVEAALLDTMYDLPSMEDVEKVVIDESVIDGQS | |
| | KPLLIYGKPEAQQASGE | |
| N-terminus BP- | MGKRTADGSEFESPKKKRKVGSGMTDKRKDGSGKLLYCSFCGKS | 2 |
| NLS tagged E. coli | QHEVRKLIAGPSVYICDECVDLCNDIIREEIKEVAPHRERSALPTPH | |
| derived ClpX | EIRNHLDDYVIGQEQAKKVLAVAVYNHYKRLRNGDTSNGVELGK | |
| | SNILLIGPTGSGKTLLAETLARLLDVPFTMADATTLTEAGYVGEDV | |
| | ENIIQKLLQKCDYDVQKAQRGIVYIDEIDKISRKSDNPSITRDVSGE | |
| | GVQQALLKLIEGTVAAVPPQGGRKHPQQEFLQVDTSKILFICGGAF | |
| | AGLDKVISHRVETGSGIGFGATVKAKSDKASEGELLAQVEPEDLIK | |
| | FGLIPEFIGRLPVVATLNELSEEALIQILKEPKNALTKQYQALFNLE | |
| | GVDLEFRDEALDAIAKKAMARKTGARGLRSIVEAALLDTMYDLP | |
| | SMEDVEKVVIDESVIDGQSKPLLIYGKPEAQQASGE | |
| C-terminus BP- | MGMTDKRKDGSGKLLYCSFCGKSQHEVRKLIAGPSVYICDECVD | 3 |
| NLS tagged E. coli | LCNDIIREEIKEVAPHRERSALPTPHEIRNHLDDYVIGQEQAKKVL | |
| derived ClpX | AVAVYNHYKRLRNGDTSNGVELGKSNILLIGPTGSGKTLLAETLA | |
| | RLLDVPFTMADATTLTEAGYVGEDVENIIQKLLQKCDYDVQKAQ | |
| | RGIVYIDEIDKISRKSDNPSITRDVSGEGVQQALLKLIEGTVAAVPP | |
| | QGGRKHPQQEFLQVDTSKILFICGGAFAGLDKVISHRVETGSGIGF | |
| | GATVKAKSDKASEGELLAQVEPEDLIKFGLIPEFIGRLPVVATLNE | |
| | LSEEALIQILKEPKNALTKQYQALFNLEGVDLEFRDEALDAIAKKA | |
| | MARKTGARGLRSIVEAALLDTMYDLPSMEDVEKVVIDESVIDGQS | |
| | KPLLIYGKPEAQQASGEGSGKRTADGSEFESPKKKRKV | |
| N-terminus BP- | MGKRTADGSEFESPKKKRKVGSGMTDKRKDGSGKLLYCSFCGKS | 4 |
| NLS tagged human | QHEVRKLIAGPSVYICDECVDLCNDIIREEIKEVAPHRERSALPTPH | |
| codon optimized E. | EIRNHLDDYVIGQEQAKKVLAVAVYNHYKRLRNGDTSNGVELGK | |
| coli derived ClpX | SNILLIGPTGSGKTLLAETLARLLDVPFTMADATTLTEAGYVGEDV | |
| | ENIIQKLLQKCDYDVQKAQRGIVYIDEIDKISRKSDNPSITRDVSGE | |
| | GVQQALLKLIEGTVAAVPPQGGRKHPQQEFLQVDTSKILFICGGAF | |
| | AGLDKVISHRVETGSGIGFGATVKAKSDKASEGELLAQVEPEDLIK | |
| | FGLIPEFIGRLPVVATLNELSEEALIQILKEPKNALTKQYQALFNLE | |
| | GVDLEFRDEALDAIAKKAMARKTGARGLRSIVEAALLDTMYDLP | |
| | SMEDVEKVVIDESVIDGQSKPLLIYGKPEAQQASGE | |
| Pseudoalteromonas | MGMSDTPTDGDKSNKLLYCSFCGKSQHEVRKLIAGPSVYICDECV | 5 |
| sp. derived ClpX | ELCNDIIREEIKDIAPKHNSSDKLPVPKEIRNHLDDYVIGQDHAKK | |
| | VLSVAVYNHYKRLRNQSTKQEVELGKSNILLIGPTGSGKTLLAET | |
| | LARLLDVPFTMADATTLTEAGYVGEDVENIIQKLLQKCDYDVEK | |
| | AQRGIVYIDEIDKISRKSDNPSITRDVSGEGVQQALLKLIEGTVASV | |
| | PPQGGRKHPQQEFLQVDTSKILFICGGAFAGLDKVIEQRSHKNTGI | |
| | GFGVNVKESASSRSLSETFKDVEPEDLVKYGLIPEFIGRLPVVATLT | |
| | ELDEAALVQILSEPKNAITKQFSVLFGMEDVELEFRDDALSAIAHK | |
| | AMERKTGARGLRSIVEGVLLDTMYELPSMDDVSKVVIDETVIKGE | |
| | SDPILIYENNNQDKAASE | |
| N-terminus BP- | MGKRTADGSEFESPKKKRKVGSGMSDTPTDGDKSNKLLYCSFCG | 6 |
| NLS tagged human | KSQHEVRKLIAGPSVYICDECVELCNDIIREEIKDIAPKHNSSDKLP | |
| codon optimized | VPKEIRNHLDDYVIGQDHAKKVLSVAVYNHYKRLRNQSTKQEVE | |
| Pseudoalteromonas | LGKSNILLIGPTGSGKTLLAETLARLLDVPFTMADATTLTEAGYVG | |
| sp. derived ClpX | EDVENIIQKLLQKCDYDVEKAQRGIVYIDEIDKISRKSDNPSITRDV | |
| | SGEGVQQALLKLIEGTVASVPPQGGRKHPQQEFLQVDTSKILFICG | |
| | GAFAGLDKVIEQRSHKNTGIGFGVNVKESASSRSLSETFKDVEPED | |
| | LVKYGLIPEFIGRLPVVATLTELDEAALVQILSEPKNAITKQFSVLF | |
| | GMEDVELEFRDDALSAIAHKAMERKTGARGLRSIVEGVLLDTMY | |
| | ELPSMDDVSKVVIDETVIKGESDPILIYENNNQDKAASE | |
| Vibrio cholerae | MGMTDKSKEGGSSKLLYCSFCGKSQHEVRKLIAGPSVYICDECVD | 7 |
| derived ClpX | LCNDIIREEIKDVLPKKESAALPTPRKIREHLDDYVIGQEHAKKVL | |
| | AVAVYNHYKRLRNGDTTSEGVELGKSNILLIGPTGSGKTLLAETL | |
| | ARLLDVPFTMADATTLTEAGYVGEDVENIIQKLLQKCDYDVAKA | |
| | ERGIVYIDEIDKISRKSENPSITRDVSGEGVQQALLKLIEGTVASVPP | |
| | QGGRKHPQQEFLQVDTSKILFICGGAFAGLDKVIEQRVATGTGIGF | |
| | GADVRSKDNSKTLSELFTQVEPEDLVKYGLIPEFIGRLPVTATLTE | |
| | LDEEALIQILCEPKNALTKQYAALFELENVDLEFREDALKAIAAKA | |
| | MKRKTGARGLRSILEAVLLETMYELPSMEEVSKVVIDESVINGES | |
| | APLLIYSANESQAAGAE | |
| N-terminus BP- | MGKRTADGSEFESPKKKRKVGSGMTDKSKEGGSSKLLYCSFCGK | 8 |
| NLS tagged human | SQHEVRKLIAGPSVYICDECVDLCNDIIREEIKDVLPKKESAALPTP | |
| codon optimized | RKIREHLDDYVIGQEHAKKVLAVAVYNHYKRLRNGDTTSEGVEL | |
| Vibrio cholerae | GKSNILLIGPTGSGKTLLAETLARLLDVPFTMADATTLTEAGYVGE | |
| derived ClpX | DVENIIQKLLQKCDYDVAKAERGIVYIDEIDKISRKSENPSITRDVS | |
| | GEGVQQALLKLIEGTVASVPPQGGRKHPQQEFLQVDTSKILFICGG | |
| | AFAGLDKVIEQRVATGTGIGFGADVRSKDNSKTLSELFTQVEPED | |
| | LVKYGLIPEFIGRLPVTATLTELDEEALIQILCEPKNALTKQYAALF | |
| | ELENVDLEFREDALKAIAAKAMKRKTGARGLRSILEAVLLETMYE | |
| | LPSMEEVSKVVIDESVINGESAPLLIYSANESQAAGAE | |
| N-terminus BP- | MGKRTADGSEFESPKKKRKVGSGMLNQELELSLNMAFARAREHR | 9 |
| NLS tagged E. coli | HEFMTVEHLLLALLSNPSAREALEACSVDLVALRQELEAFIEQTTP | |
| derived ClpA | VLPASEEERDTQPTLSFQRVLQRAVFHVQSSGRNEVTGANVLVAI | |
| | FSEQESQAAYLLRKHEVSRLDVVNFISHGTRKDEPTQSSDPGSQPN | |
| | SEEQAGGEERMENFTTNLNQLARVGGIDPLIGREKELERAIQVLCR | |
| | RRKNNPLLVGESGVGKTAIAEGLAWRIVQGDVPEVMADCTIYSL | |
| | DIGSLLAGTKYRGDFEKRFKALLKQLEQDTNSILFIDEIHTIIGAGA | |
| | ASGGQVDAANLIKPLLSSGKIRVIGSTTYQEFSNIFEKDRALARRFQ | |
| | KIDITEPSIEETVQIINGLKPKYEAHHDVRYTAKAVRAAVELAVKY | |
| | INDRHLPDKAIDVIDEAGARARLMPVSKRKKTVNVADIESVVARI | |
| | ARIPEKSVSQSDRDTLKNLGDRLKMLVFGQDKAIEALTEAIKMAR | |
| | AGLGHEHKPVGSFLFAGPTGVGKTEVTVQLSKALGIELLRFDMSE | |
| | YMERHTVSRLIGAPPGYVGFDQGGLLTDAVIKHPHAVLLLDEIEK | |
| | AHPDVFNILLQVMDNGTLTDNNGRKADFRNVVLVMTTNAGVRE | |
| | TERKSIGLIHQDNSTDAMEEIKKIFTPEFRNRLDNIIWFDHLSTDVIH | |
| | QVVDKFIVELQVQLDQKGVSLEVSQEARNWLAEKGYDRAMGAR | |
| | PMARVIQDNLKKTLANELLFGSLVDGGQVTVALDKEKNELTYGF | |
| | QSAQKHKAEAAH | |
| N-terminus BP- | MGKRTADGSEFESPKKKRKVGSGMRLDRLTNKFQLALADAQSLA | 10 |
| NLS tagged E. coli | LGHDNQFIEPLHLMSALLNQEGGSVSPLLTSAGINAGQLRTDINQA | |
| derived ClpB | LNRLPQVEGTGGDVQPSQDLVRVLNLCDKLAQKRGDNFISSELFV | |
| | LAALESRGTLADILKAAGATTANITQAIEQMRGGESVNDQGAEDQ | |
| | RQALKKYTIDLTERAEQGKLDPVIGRDEEIRRTIQVLQRRTKNNPV | |
| | LIGEPGVGKTAIVEGLAQRIINGEVPEGLKGRRVLALDMGALVAG | |
| | AKYRGEFEERLKGVLNDLAKQEGNVILFIDELHTMVGAGKADGA | |
| | MDAGNMLKPALARGELHCVGATTLDEYRQYIEKDAALERRFQK | |
| | VFVAEPSVEDTIAILRGLKERYELHHHVQITDPAIVAAATLSHRYIA | |
| | DRQLPDKAIDLIDEAASSIRMQIDSKPEELDRLDRRIIQLKLEQQAL | |
| | MKESDEASKKRLDMLNEELSDKERQYSELEEEWKAEKASLSGTQ | |
| | TIKAELEQAKIAIEQARRVGDLARMSELQYGKIPELEKQLEAATQL | |
| | EGKTMRLLRNKVTDAEIAEVLARWTGIPVSRMMESEREKLLRME | |
| | QELHHRVIGQNEAVDAVSNAIRRSRAGLADPNRPIGSFLFLGPTGV | |
| | GKTELCKALANFMFDSDEAMVRIDMSEFMEKHSVSRLVGAPPGY | |
| | VGYEEGGYLTEAVRRRPYSVILLDEVEKAHPDVFNILLQVLDDGR | |
| | LTDGQGRTVDFRNTVVIMTSNLGSDLIQERFGELDYAHMKELVLG | |
| | VVSHNFRPEFINRIDEVVVFHPLGEQHIASIAQIQLKRLYKRLEERG | |
| | YEIHISDEALKLLSENGYDPVYGARPLKRAIQQQIENPLAQQILSGE | |
| | LVPGKVIRLEVNEDRIVAVQ | |
| N-terminus BP- | MGKRTADGSEFESPKKKRKVGSGMSYSGERDNFAPHMALVPMVI | 11 |
| NLS tagged E. coli | EQTSRGERSFDIYSRLLKERVIFLTGQVEDHMANLIVAQMLFLEAE | |
| derived ClpP | NPEKDIYLYINSPGGVITAGMSIYDTMQFIKPDVSTICMGQAASMG | |
| | AFLLTAGAKGKRFCLPNSRVMIHQPLGGYQGQATDIEIHAREILKV | |
| | KGRMNELMALHTGQSLEQIERDTERDRFLSAPEAVEYGLVDSILT | |
| | HRN | |

TABLE 3
Guide RNA sequences

| Guide | Target | | | SEQ |
|---|---|---|---|---|
| RNA ID | ID | Description | Target Sequence | ID NO |
| crRNA 1 | tSL0264 | | AATGCAGGCAAATCAACCTTAGTCTGAAGGCC | 103 |
| crRNA 2 | tSL0263 | | TTAAGAAGTAAGTTGTGTTCTTCTTTGCCTAG | 104 |
| crRNA 3 | tSL0365 | | TCAGGAGACTGTGTAACACCAATGCAGGCAAA | 105 |
| crRNA 4 | tSL0265 | | TAGGCAAAGAAGAACACAACTTACTTCTTAAG | 106 |
| crRNA 5 | tSL0366 | | TTGCGACGTAGGGATAACAGGGTAATCCTCAG | 107 |
| crRNA 6 | tSL0367 | | CTTGCGACGTAGGGATAACAGGGTAATCCTCA | 108 |
| crRNA 7 | tSL0368 | | AGTGTGATGGATATCTGCAGAATTCGCCCTTG | 109 |
| sgRNA 8 | tSL0435 | TTN sgRNA 4 | ATGAGCTCTCTTCAACGTTA | 110 |
| sgRNA 9 | tSL0434 | TTN sgRNA 3 | GGGCACAGTCCTCAGGTTTG | 111 |
| sgRNA 10 | tSL0433 | TTN sgRNA 2 | ATGTTAAAATCCGAAAATGC | 112 |
| sgRNA 11 | tSL0447 | TTN sgRNA 1 | CCTTGGTGAAGTCTCCTTTG | 113 |
| crRNA 12 | tSL0378 | TTN crRNA 4 | AAGGAAATAGAACTGTATTTAAAGAATAACTG | 114 |
| crRNA 13 | tSL0377 | TTN crRNA 3 | TCAAAGGAGACTTCACCAAGGAAATAGAACTG | 115 |
| crRNA 14 | tSL0375 | TTN crRNA 1; | TTGGTGAAGTCTCCTTTGAGGTACTAAATTTA | 116 |
| | | ChIP T crRNA | | |
| crRNA 15 | tSL0376 | TTN crRNA 2 | TTTGAGGTACTAAATTTAGCACTGTCAATCAG | 117 |
| crRNA 16 | tSL0416 | MIAT crRNA 4 | AAGGGCTTAACCAGGAAGACCTCGGGTGTATG | 118 |
| crRNA 17 | tSL0415 | MIAT crRNA 3 | GTCCACATTAGGCCGCAGAGAGCTCAGGGCTG | 119 |
| crRNA 18 | tSL0414 | MIAT crRNA 2 | GTCCACATTAGGCCGCAGAGAGCTCAGGGCTG | 120 |
| crRNA 19 | tSL0413 | MIAT crRNA 1 | GCTCCCGCATTAAAATTTCATGGGCGCTGCAG | 121 |
| crRNA 20 | tSL0420 | ASCL1 crRNA 4 | GAGGAGTGGTTGTGAGCCGTCCTGTAGGTGGG | 122 |
| crRNA 21 | tSL0419 | ASCL1 crRNA 3 | TCGGTGACCCTAGAAATTGGAGCAAATTACGA | 123 |
| crRNA 22 | tSL0418 | ASCL1 crRNA 2 | CTGCGCTTTGCTTCAAGTTCTTAGTAGAATCC | 124 |
| crRNA 23 | tSL0417 | ASCL1 crRNA 1 | CCTCCCGTTCCTTTCTCCCGCTCCTTGCAAAC | 125 |
| crRNA 24 | tSL0423 | ACTC1 crRNA 3 | TGAATGGCTTTACTCAGAGAGCTGGTGCTGGG | 126 |
| crRNA 25 | tSL0422 | ACTC1 crRNA 2 | ACTCAGATGTGCTGCTGCGGTGTCCTTTGTGC | 127 |
| crRNA 26 | tSL0421 | ACTC1 crRNA 1 | TCAGCAGAGGGCAGGGCGCCAAGCCTTCCCAC | 128 |
| sgRNA 27 | tSL0436 | MIAT sgRNA 1 | GCGCCCATGAAATTTTAATG | 129 |
| sgRNA 28 | tSL0437 | MIAT sgRNA 2 | ATGCGGGAGGCTGAGCGCAC | 130 |
| sgRNA 29 | tSL0438 | MIAT sgRNA 3 | CATTAGGCCGCAGAGAGCTC | 131 |
| sgRNA 30 | tSL0439 | MIAT sgRNA 4 | GCTTCTGCGCCCCTGGTCCG | 132 |
| sgRNA 31 | tSL0440 | ASCL1 sgRNA 1 | CGGGAGAAAGGAACGGGAGG | 133 |
| sgRNA 32 | tSL0441 | ASCL1 sgRNA 2 | AAGAACTTGAAGCAAAGCGC | 134 |
| sgRNA 33 | tSL0442 | ASCL1 sgRNA 3 | TCCAATTTCTAGGGTCACCG | 135 |
| sgRNA 34 | tSL0443 | ASCL1 sgRNA 4 | GTTGTGAGCCGTCCTGTAGG | 136 |
| sgRNA 35 | tSL0444 | ACTC1 sgRNA 1 | TGGCGCCCTGCCCTCTGCTG | 137 |
| sgRNA 36 | tSL0445 | ACTC1 sgRNA 2 | ACCGCAGCAGCACATCTGAG | 138 |
| sgRNA 37 | tSL0446 | ACTC1 sgRNA 3 | AATGGCTTTACTCAGAGAGC | 139 |
| crRNA 38 | tSL0105 | NT crRNA for | GCGAGGTATTCGGCTCCGCGACGGAGGCTAAG | 140 |
| | | activation, | | |
| | | ChIP, and | | |
| | | integration | | |
| | | assays | | |
| crRNA 39 | tSL0396 | | AGGGGTCCGAGAGCTCAGCTAGTCTTCTTCCT | 141 |
| crRNA 40 | tSL0394 | | GAGCTGGGACCACCTTATATTCCCAGGGCCGG | 142 |
| crRNA 41 | tSL0424 | | CAGGGCCGGTTAATGTGGCTCTGGTTCTGGGT | 143 |
| crRNA 42 | tSL0426 | | CCTCCACCCCACAGTGGGGCCACTAGGGACAG | 144 |
| crRNA 43 | tSL0425 | | ACAGTGGGGCCACTAGGGACAGGATTGGTGAC | 145 |
| crRNA 44 | tSL0427 | | TTAGGCCTCCTCCTTCCTAGTCTCCTGATATT | 146 |
| crRNA 45 | tSL0392 | | TGTTAGGCAGATTCCTTATCTGGTGACACACC | 147 |
| AAVS1-1 | tSL0425 | | ACAGTGGGGCCACTAGGGACAGGATTGGTGAC | 148 |
| AAVS1-2 | tSL0394 | | GAGCTGGGACCACCTTATATTCCCAGGGCCGG | 149 |
| AAVS1-3 | tSL0533 | | CCAGGGTGTGCTGGGCAGGTCGCGGGGAGCGC | 150 |
| HEK3-1 | tSL0428 | | CTGCTTCCTCCAGAGGGCGTCGCAGGACAGCT | 151 |
| ACTB-1 | tSL0455 | | CGGAGCTGCGCCCTTTCTCACTGGTTCTCTCT | 152 |
| ACTB-2 | tSL0456 | | GTAGGACTCTCTTCTCTGACCTGAGTCTCCTT | 153 |
| ACTB-3 | tSL0457 | | ATGAGGCTGGTGTAAAGCGGCCTTGGAGTGTG | 154 |
| CANX-1 | tSL0458 | | TCTCCTCACTGTGCCCTGAAAAGTATTTCTTA | 155 |
| CANX-2 | tSL0459 | | TTTAGGGAGCTTAAATTCTACTTGGGGGAAAC | 156 |
| CANX-3 | tSL0460 | | ATTGCTTACTAAAGTCCTTTACCCAGCACCTC | 157 |
| CBX1-1 | tSL0461 | | CACAATTCAAACTACTGTCAAAGTAGTTTTGT | 158 |
| CBX1-2 | tSL0462 | | TGAAATCTTAGGTAGGCTAATGCCTACAAAGT | 159 |
| CBX1-3 | tSL0463 | | AAAGGATTCTAACAGCTCTCTTACTTGAGCCA | 160 |
| VIM-1 | tSL0464 | | TGTGCTCCAGAATTAGTGATTTGCTTTGGTGC | 161 |
| QARS1-1 | tSL0474 | | TGGCAAGCAAGATGACCACTTGCTGTTCCCAT | 162 |
| OXA1L-1 | tSL0475 | | TGACCCAGTGAACCAGGCCCCAGGACAGCTCG | 163 |
| OXA1L-2 | tSL0476 | | TGCTCACCGGGACCTGAATGTCATGACCTCGG | 164 |
| OXA1L-3 | tSL0477 | | TGGCGAGAGCACCTCGGCCTCGTTCTCAGGGC | 165 |
| NT | tSL0000 | | GTTGTCTGACACTTGTCACAAACCGCTAGGAG | 166 |
| T | tSL0004 | | AGTACAGCGCGGCTGAAATCATCATTAAAGCG | 167 |

The scope of the present invention is not limited by what has been specifically shown and described hereinabove. Those skilled in the art will recognize that there are suitable alternatives to the depicted examples of materials, configurations, constructions, and dimensions. Variations, modifications, and other implementations of what is described herein will occur to those of ordinary skill in the art without departing from the spirit and scope of the invention.

Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety.

1. A system for RNA-guided DNA modification, comprising:
a) an engineered Clustered Regularly Interspaced Short Palindromic Repeats (CRISPR)-associated transposon (CAST) system or one or more nucleic acids encoding the engineered CAST system, wherein the CAST system comprises at least one or all of:
i) at least one Cas protein;
ii) at least one transposon-associated protein; and
iii) at least one guide RNA (gRNA) complementary to at least a portion of a target nucleic acid sequence; and
b) at least one unfoldase protein, or a nucleic acid encoding thereof.

2. The system of claim 1, wherein the at least one Cas protein is derived from a Type I CRISPR-Cas system or a Type V CRISPR-Cas system.

3. The system of claim 1, wherein the at least one Cas protein comprises Cas5, Cas6, Cas7, and Cas8; or Cas12k.

4. The system of claim 1, wherein the at least one transposon protein is derived from a Tn7 or Tn7-like transposon system.

5. The system of claim 1, wherein the at least one transposon-associated protein comprises TnsA, TnsB, TnsC, or a combination thereof, and optionally TnsD and/or TniQ.

6. The system of claim 1, wherein the at least one gRNA is a non-naturally occurring gRNA.

7. The system of claim 1, wherein the at least one unfoldase protein comprises ClpX, or a homolog thereof.

8. The system of claim 1, wherein the at least one unfoldase protein is derived from same or different organism as that of the engineered CAST system.

9. The system of claim 1, wherein the one or more nucleic acids encoding the engineered CAST system comprises one or more messenger RNAs, one or more vectors, or a combination thereof.

10. A composition comprising the system of claim 1.

11. A cell comprising the system of claim 1.

12. A method for DNA integration, comprising contacting a target nucleic acid sequence with the system of claim 1 or a composition comprising thereof.

13. The method of claim 12, wherein the target nucleic acid sequence is in a cell and the contacting a target nucleic acid sequence comprises introducing the system into the cell.

14. The method of claim 13, wherein the cell is a prokaryotic cell or a eukaryotic cell.

15. The method of claim 13, wherein the introducing the system into the cell comprises administering the system to a subject.
